(19) United States
(12) Patent Application Publication
(10) Pub. No.: US 2024/0327804 A1
(43) Pub. Date: Oct. 3, 2024

(54) CONJUGATES AND METHODS OF USE THEREOF

(71) Applicant: Regents of the University of Minnesota, Minneapolis, MN (US)

(72) Inventors: Carston R. Wagner, Minneapolis, MN (US); Yiao Wang, Minneapolis, MN (US); Mark Distefano, Minneapolis, MN (US)

(73) Assignee: Regents of the University of Minnesota, Minneapolis, MN (US)

(21) Appl. No.: 18/593,314

(22) Filed: Mar. 1, 2024

Related U.S. Application Data
(60) Provisional application No. 63/449,891, filed on Mar. 3, 2023.

(51) Int. Cl. C12N 9/06; A61K 47/64; G01N 33/50

(52) U.S. Cl. CPC C12N 9/003; A61K 47/64; G01N 33/5032; C07K 2319/035; G01N 2333/90666

(57) ABSTRACT

Certain embodiments of the invention provide conjugates, chemically self-assembled nanoring (CSAN), and cell modified with CSAN as described herein. Certain embodiments of the invention provide a method for cell-based drug delivery. Certain embodiments of the invention provide a method of transferring a cargo from a sender cell to a receiver cell as described herein.

### CROSS-REFERENCE TO RELATED APPLICATION

This application claims priority to U.S. Provisional Application No. 63/449,891 that was filed on Mar. 3, 2023. The entire content of the application referenced above is hereby incorporated by reference.

### GOVERNMENT FUNDING

This invention was made with government support under GM084152, GM141853, CA185627 and CA247681 awarded by the National Institutes of Health. The government has certain rights in the invention.

### BACKGROUND OF THE INVENTION

Cell-based therapeutics have rapidly emerged and expanded as invaluable tools in translational medicine with a significant impact on several diverse fields, including tissue engineering, regenerative medicine, and immunotherapy. The potential to augment and modulate the effects of cells on other cells is of key interest to engineering synthetic biological processes. Consequently, recent approaches have begun to be developed for the monitoring and engineering of cell-cell interactions. Cell-cell interactions have been monitored by genetically engineering cells to take advantage of surface modifying non-discriminating chemical conjugation reactions or non-genetically by microdissection methods. Genetic cargo transfer based approaches have emerged that rely on the binding of engineered receptors on receiver cells to engineered fluorescent or chemically modified proteins fused to membrane spanning domains on sender cells. In this last approach, cargo transfer has also been used to deliver proteins and nucleic acids to the receiver cells from the sender cells. Each of these approaches has proved versatile. Nevertheless, in each case genetic engineering of either the sender cell or receiver cell or both is required, which can be time-consuming, inefficient, and, in some cases, difficult, since not all cells are amenable to genetic modification. Consequently, alternative methodologies that allow for the non-genetic modification of normal cells and the evaluation of their interactions with cells expressing a variety of natural or engineered receptors would be of value. Accordingly, new compositions and methods are needed to treat and study disease such as cancer and to study cell-cell interactions.

### SUMMARY OF THE INVENTION

Certain embodiments of the invention provide a conjugate A1 comprising:
A. a fusion protein comprising
   i. a targeting domain,
   ii. a first dihydrofolate reductase (DHFR) operably linked to a second DHFR, and
   iii. a prenyltransferase substrate domain;
B. a linker A operably linked to the prenyltransferase substrate domain of the fusion protein; and
C. a therapeutic agent or a detectable agent operably linked to linker A with a linker B.

Certain embodiments of the invention provide a conjugate A1 comprising:
A. a fusion protein comprising
   i. a targeting domain,
   ii. a first dihydrofolate reductase (DHFR) operably linked to a second DHFR, and
B. a therapeutic agent or a detectable agent operably linked to the DHFR (e.g., the second DHFR) with a linker B.

Certain embodiments of the invention provide a chemically self-assembled nanoring (CSAN) comprising a plurality of conjugates as described in herein (e.g., conjugate A1) and a plurality of bisMTX compounds.

Certain embodiments of the invention provide a self-assembled nanoring (CSAN) comprising a plurality of conjugate A1 and a plurality of conjugate A2, wherein conjugate A2 comprises
A. fusion protein comprising
   i. a targeting domain,
   ii. a first dihydrofolate reductase (DHFR) operably linked to a second DHFR, and
   iii. a prenyltransferase substrate domain; and
B. a lipid operably linked to the prenyltransferase substrate domain of the fusion protein.

Certain embodiments of the invention provide a chemically self-assembled nanoring (CSAN) comprising a plurality of conjugate B1 and fusion protein B2, and a plurality of bisMTX compounds,
wherein conjugate B1 comprises:
A. fusion protein comprising
   i. a first dihydrofolate reductase (DHFR) operably linked to a second DHFR, and
   ii. a prenyltransferase substrate domain; and
B. a lipid operably linked to the prenyltransferase substrate domain of the fusion protein;
wherein conjugate B1 lacks a targeting domain; and
wherein fusion protein B2 comprises:
i. a targeting domain, and
   ii. a first dihydrofolate reductase (DHFR) operably linked to a second DHFR.

Certain embodiments of the invention provide a pharmaceutical composition comprising a conjugate as described herein and a pharmaceutically acceptable excipient.

Certain embodiments of the invention provide a pharmaceutical composition comprising a CSAN as described herein and a pharmaceutically acceptable excipient.

Certain embodiments of the invention provide a cell comprising a CSAN as described herein in the cell membrane.

Certain embodiments of the invention provide a method of intercellular delivery comprising
a) contacting a sender cell with a CSAN as described herein, and
b) contacting the CSAN modified sender cell with a receiver cell,
   wherein a therapeutic agent or a detectable agent comprised within the CSAN is delivered from the sender cell to the receiver cell. Certain embodiments of the invention provide a method for cell-based drug delivery.

Certain embodiments of the invention provide CSANs that can undergo efficient CSAN-assisted cell-cell cargo transfer (C4T) and that can target cells as well as delivery biologically active agents and detectable groups.

Certain embodiments of the invention provide a chemically self-assembled nanoring (CSAN) comprising a plurality of conjugates as described herein and a plurality of BisMTX compounds.

Certain embodiments of the invention provide a CSAN as described herein.

Certain embodiments of the invention provide a pharmaceutical composition comprising a conjugate, a CSAN, or a CSAN modified cell described herein and a pharmaceutically acceptable excipient.

Certain embodiments of the invention provide a method for treating or preventing cancer in an animal (e.g., a human) comprising administering a CSAN modified cell described herein to the animal.

Certain embodiments of the invention provide a conjugate, a CSAN, or a CSAN modified cell for use in medical therapy.

Certain embodiments of the invention provide a conjugate, a CSAN, or a CSAN modified cell for the prophylactic or therapeutic treatment of cancer.

Certain embodiments of the invention provide the use of a conjugate, a CSAN, or a CSAN modified cell to prepare a medicament for treating cancer in an animal (e.g., a human).

Certain embodiments of the invention provide a kit comprising:
1) a conjugate or a CSAN described herein;
2) instructions for assembling the conjugate to a CSAN and/or for contacting the CSAN with a cell.

Certain embodiments of the invention provide a method of using a conjugate, a CSAN, or a CSAN modified cell described herein.

The invention also provides processes and intermediates disclosed herein that are useful for preparing fusion proteins, conjugates, CSANs, and CSAN modified cells described herein.

Certain embodiments of the invention provide a nucleic acid encoding a polypeptide described herein.

Certain embodiments of the invention provide an expression cassette comprising a nucleic acid sequence as described herein and a promoter operably linked to the nucleic acid.

Certain embodiments of the invention provide a vector comprising an expression cassette described herein.

Certain embodiments of the invention provide a conjugate comprising a fusion protein described herein and a therapeutic agent or a detectable agent operably linked to (e.g., via a linker described herein) the fusion protein.

### BRIEF DESCRIPTION OF THE FIGURES

The patent or application file contains at least one drawing executed in color. Copies of this patent or patent application publication with color drawing(s) will be provided by the Office upon request and payment of the necessary fee.

FIGS. 1A-IC shows the schematic illustration of CSAN-assisted cell-cell cargo transfer to record cell-cell interactions and manipulate cell functions. FIG. 1A shows that the CSAN was formed through the self-assembly of the DHFR² fusion proteins by the chemical dimerizer, bisMTX. FIG. 1B shows that the DHFR² fusion protein that contains the targeting element and the C-terminal “CVIA” sequence can be farnesylated and oligomerized into nanorings for cell surface modification. FIG. 1C shows that the surface-bound CSANs on the f-CSAN-modified cell (sender cell) can be transferred and internalized into the target cell (receiver cell) during cell-cell interactions. Functional molecules, such as dyes, oligonucleotides, and cancer drugs can be loaded to the f-CSANs as payloads for intercellular interaction-dependent delivery.

FIGS. 2A-2G show the characterization of the CSANs and assessment of their ability for receptor binding and cell surface modification. FIG. 2A shows that αEGFR-Fn3-DHFR²-CVIA protein was efficiently farnesylated by farnesyltransferase and the proteins were characterized by LC-MS. FIG. 2B shows that the fluorescein-labeled αEGFR-Fn3-DHFR²-Far protein was oligomerized into CSANs and the hydrodynamic diameter of the CSANs was measured by DLS. FIG. 2C shows that the fluorescein-labeled αEGFR-Fn3-Far CSANs were imaged by Cryo-TEM. Scale bar, 10 nm. FIG. 2D shows that the selectivity of the CSANs to their respective cell surface receptors was verified by flow cytometry, where the DHFR² proteins were labeled with fluorescein for detection and the non-targeting DHFR²-CVIA CSANs served as the control. FIG. 2E shows that f-CSANs were shown to universally modify mammalian cell surface by flow cytometry, where the DHFR² proteins were labeled with fluorescein for detection and the unfarnesylated αEGFR-Fn3 CSANs served as the control.

FIG. 2F shows that the cell surface modification of Raji cells by f-CSANs were imaged by fluorescent microscopy. Scale bar, 5 μm. FIG. 2G shows that the stability studies of the f-CSANs on cell surface were conducted by co-culturing the CSAN-modified Raji cells and the CFSE-stained Raji cells at a 6:4 ratio in 1 mL of the medium for 48 h. The data show a negligible level of non-specific cell-cell CSAN migration over 24 h and 48 h. CSANs were labeled with DyLight-650 for detection. For FIG. 2C and FIG. 2G, data are represented as mean values±SD (from n=3 independent experimental replicates). In some instances, small error bars are obscured by the symbols denoting the mean value.

FIGS. 3A-3I show that the cell-cell CSAN transfer is dependent on ligand-receptor binding and cell-cell physical contacts. FIG. 3A shows that the representative flow cytometry plots demonstrate the proportions of cells during the co-culture of the CSAN-modified Raji cells and the A431-R cells. The percentage of CSAN⁺/mKate⁺ double-positive cell population is shown in the plot and indicates the CSAN transfer from Raji cells to A431-R cells. The quantitative data of this flow cytometry study is presented in FIG. 3B, the bar graphs of percent cell populations in the cell-cell CSAN transfer study for the non-targeting DHFR²-Far CSANs and αEGFR-Fn3-Far CSANs. FIG. 3C shows the Schematic illustration of the transwell assay, where the transwell inserts were used to separate the CSAN-modified Raji cells from the A431-R cells during the co-culture. The A431-R cells were co-cultured with the CSAN-modified Raji cells at a 1:1 ratio with or without the transwell inserts at 37° C. for 30 min and analyzed by flow cytometry. FIG. 3D shows that the representative flow cytometry histograms of the transwell assay indicate the cell-cell CSAN transfer is dependent on cell-cell physical contacts. The quantitative flow cytometry data of this transwell assay is presented in FIG. 3E. FIG. 3F shows that the representative flow cytometry plots of the competition assay demonstrate CSAN transfer is dependent on receptor binding. The quantitative flow cytometry data of this competition assay is presented in the FIG. 3G. FIG. 3H illustrates the time-lapse images that show the process of the CSAN transfer from Raji cells to A431-R cells. The A431-R cells expressed red fluorescent mKate protein in the nucleus as a marker and CSANs are shown in green. The arrows highlight the punctuate spots of the CSANs. Scale bar, 5 μm. FIG. 3I illustrates the flow cytometry study of CSAN transfer that shows that the CSAN-modified Raji cells are capable of labeling multiple folds of A431-R cells through cell-cell CSAN transfer. For FIGS. 3B, 3E, 3G and 3I data are represented as mean values±SD (from n=3 independent experimental replicates). In some instances, small error bars are obscured by the symbols denoting the mean value. Significance in FIG. 3E was tested using a two-tailed, unpaired t-test and is indicated as ****P<0.0001.

FIGS. 4A-4I show the kinetics study of the cell-cell CSAN transfer for different cell lines and receptors. FIG. 4A shows the representative flow cytometry plots of the transfer kinetics of αEGFR-Fn3-Far CSANs from Raji cells to A431-R cells. The quantitative flow cytometry data of this kinetics study was presented in FIG. 4B. FIG. 4C shows the kinetics study for the αEGFR-Fn3-Far CSANs transferring from Raji cells to MDA-MB-231-R cells. FIG. 4D shows the kinetics study for the αHER2-afb-Far CSANs transferring from Raji cells to SK-BR-3-R cells. FIG. 4E shows the kinetics study for the αEpCAM-Fn3-Far CSANs transferring from Raji cells to HT29-R cells. FIG. 4F shows the schematic illustration of the formation of the hybrid αCD133-scFv-far CSANs. FIG. 4G shows that the hybrid αCD133-scFv-far CSANs were shown to adequately modify Raji cell surface. FIG. 4H shows the kinetics study for the hybrid αCD133-scFv-far CSANs transferring from Raji cells to HT29-R cells. FIG. 4I shows the plot of transfer kinetics of the CSANs for different target cells. The percent CSAN transfer is determined by the percentage of the CSAN^{+/}mKate⁺ cell proportion out of the mKate⁺ receiver cell proportion. For FIGS. 4B-4E, 4H and 4I, data are represented as mean values±SD (from n=3 independent experimental replicates). In some instances, small error bars are obscured by the symbols denoting the mean value.

FIGS. 5A-5L show recording cell-cell interactions by CSAN-assisted cell-cell cargo transfer. FIG. 5A illustrates the representative flow cytometry histograms that show the CSANs on the A431-R receiver cells after the co-culture with CSAN-modified T cells. The quantitative flow cytometry data are presented in FIG. 5B. FIG. 5C shows that the T-cancer cell-cell interactions were recorded by fluorescence microscopy with the C4T approach. FIG. 5D illustrates the representative flow cytometry histograms that show the CSANs on the A431-R receiver cells after the co-culture with CSAN-modified NK92 cells. The quantitative flow cytometry data are presented in FIG. 5E. FIG. 5F shows that the NK-cancer cell-cell interactions were recorded by fluorescence microscopy with the C4T approach. FIG. 5G illustrates the representative flow cytometry histograms that show the CSANs on the SK-BR-3-R receiver cells after the co-culture with CSAN-modified HUVEC cells. The quantitative flow cytometry data are presented in FIG. 5H. FIG. 5I shows that the endothelial-cancer cell-cell interactions were recorded by fluorescence microscopy with the C4T approach. FIG. 5J illustrates the representative flow cytometry histograms that show the CSANs on the RAW 264.7 receiver cells after the co-culture with CSAN-modified J558L cells. The quantitative flow cytometry data are presented in FIG. 5K. FIG. 5L shows the myeloma-macrophage cell-cell interactions were recorded by fluorescence microscopy with the C4T approach. In the flow cytometry study and the imaging study, A431-R cells and SK-BR-3-R cells expressed red fluorescent mKate protein in the nucleus as the marker, and RAW 264.7 cells were stained with the Hoechst dye and are shown in blue. All CSANs are shown in green. For FIGS. 5C, 5F FI and 5L, the arrows highlight the punctuate spots of the CSANs. Scale bars, 5 μm. For FIGS. 5B, 5E, 5H, and 5K, data are represented as mean values±SD (from n=3 independent experimental replicates). In some instances, small error bars are obscured by the symbols denoting the mean value. Significance in these plots was tested using a two-tailed, unpaired t-test and is indicated as *P<0.05, ***P<0.001 and ****P<0.0001.

FIGS. 6A-6F show the study of the cell-cell interaction-dependent delivery of the anti-cancer drug by the C4T approach. FIG. 6A shows a Schematic illustration of the formation of the hybrid αEGFR-Fn3-Far-MMAE CSANs. FIG. 6B shows the flow cytometry study of the cell-cell interaction-dependent delivery of MMAE by the C4T approach. The αEGFR-Fn3-DHFR²-MMAE protein conjugate was labeled with fluorescein for detection. FIG. 6C shows that the fluorescent images indicate the transfer of the hybrid αEGFR-Fn3-Far-MMAE CSANs from Raji cells to A431-R cells. The A431-R cells expressed red fluorescent mKate protein in the nucleus as a marker and CSANs are shown in green. Scale bar, 5 μm. The cytotoxicity of the cell-cell interaction-dependent delivery of MMAE by the C4T approach was studied using IncuCyte for (FIG. 6D) A431-R cells, (FIG. 6E) MDA-MB-231-R cells, and (FIG. 6F) MDA-MB-453-R cells, where the CSAN-modified Raji cells were co-cultured with the target cancer cells at a 3:1 ratio in the plates at 37° C. for 2 h, followed by medium exchange to remove the Raji cells. The cancer cells were returned to the IncuCyte and cultured for 4 days to quantify cell viability. For FIG. 6B and FIG. 6D-6F, data are represented as mean values±SD (from n=3 independent experimental replicates). In some instances, small error bars are obscured by the symbols denoting the mean value. Significance in (b) and (d)-(f) was tested using a two-tailed, unpaired t-test and is indicated as ****P<0.0001 and ***P<0.001.

FIG. 7A-7D shows the study of the cell-cell interaction-dependent delivery of oligonucleotides by the C4T approach. FIG. 7A shows the schematic illustration of the formation of the hybrid αEGFR-Fn3-Far-ssDNA CSANs. FIG. 7B shows the flow cytometry study of the cell-cell interaction-dependent delivery of ssDNA by the C4T approach. The αEGFR-Fn3-Far-ssDNA-AF488 protein conjugate has an AlexaFluor-488 dye for detection. FIG. 7C illustrates the representative western blot image that shows the specific knockdown of eIF4E in MDA-MB-231 cells by the interaction-dependent delivery of the anti-eIF4E antisense ssDNA using the C4T approach. Lane 1: Raji cells modified with the hybrid DHFR²-Far-KDssDNA CSANs; lane 2: Raji cells modified with the hybrid αEGFR-Fn3-Far-CTRLssDNA CSANs; lane 3: untreated control; lane 4: Raji cells modified with the hybrid αEGFR-Fn3-Far-KDssDNA CSANs. FIG. 7D shows the quantitative western bolt data summarized in the bar graph and are represented as mean values±SD (from n=3 independent biological replicates). In some instances, small error bars are obscured by the symbols denoting the mean value. Significance in (b) and (d) was tested using a two-tailed, unpaired t-test and is indicated as *P<0.05, **P<0.01, and ****P<0.0001.

FIGS. 8A-8B show the LC-MS spectra of DHFR²-CVIA protein and DHFR²-Far protein. LC-MS (Orbitrap Elite) was used to characterize the mass of (FIG. 8A) DHFR2-CVIA protein and (FIG. 8B) DHFR²-Far protein, which confirmed the farnesylation of the DHFR2-CVIA protein.

FIGS. 9A-9B show the LC-MS spectra of αHER2-afb-DHFR²-CVIA protein and αHER2-afb-DHFR²-Far protein. LC-MS(Orbitrap Elite) was used to characterize the mass of (FIG. 9A) αHER2-afb-DHFR2-CVIA protein and (FIG. 9B) αHER2-afb-DHFR²-Far protein, which confirmed the farnesylation of the αHER2-afb-DHFR2-CVIA protein.

FIGS. 10A-10B show the LC-MS spectra of αEpCAM-Fn3-DHFR²-CVIA protein and αEpCAM-Fn3-DHFR²-Far protein. LC-MS (Orbitrap Elite) was used to characterize the mass of (FIG. 10A) αEpCAM-Fn3-DHFR2-CVIA protein and (FIG. 10B) αEpCAM-Fn3-DHFR²-Far protein, which confirmed the farnesylation of the αEpCAM-Fn3-DHFR²-CVIA protein.

FIGS. 11A-11B show the SDS-PAGE gel of the fluorescein-labeled DHFR² fusion proteins. FIG. 11A shows the fluorescein-labeled DHFR² fusion proteins characterized by gel electrophoresis, and that the fluorescent labeling of the proteins was confirmed by fluorescent scanning of the gel (FITC channel). FIG. 11B show the SDS-PAGE gel of the fluorescein-labeled DHFR² fusion proteins that were then stained by Coomassie brilliant blue and imaged. Lane 1: fluorescein-labeled αEGFR-Fn3-DHFR²-CVIA; lane 2: fluorescein-labeled αEGFR-Fn3-DHFR²-Far; lane 3: fluorescein-labeled αHER2-afb-DHFR²-CVIA; lane 4: fluorescein labeled αHER²-afb-DHFR²-Far; lane 5: fluorescein-labeled αEpCAM-Fn3-DHFR²-CVIA; lane 6: fluorescein-labeled αEpCAM-Fn3-DHFR²-Far; lane 7: fluorescein-labeled DHFR²-CVIA; lane 8: fluorescein-labeled DHFR2-Far; lane 9: fluorescein-labeled αCD133-scFv-DHFR².

FIGS. 12A-12C show the hydrodynamic diameters of the farnesylated CSANs measured by dynamic light scattering analysis. The dynamic light scattering was used to measure the hydrodynamic diameter of FIG. 12A, fluorescein-labeled αHER2-afb-Far CSANs (31.7±8.7 nm), FIG. 12B, fluorescein-labeled αEpCAM-Fn3-Far CSANs (33.8±7.4 nm), and FIG. 12C, fluorescein-labeled DHFR2-Far CSANs (23.4±8.4 nm).

FIGS. 13A-13C show the cryo-TEM imaging of the farnesylated CSANs. FIG. 13A shows the fluorescein-labeled αHER2-afb-Far protein, FIG. 13B shows the fluorescein-labeled αEpCAM-Fn3-Far protein, and FIG. 13C shows the fluorescein-labeled DHFR²-Far protein was self-assembled into the corresponding nanorings by bisMTX and the CSANs were characterized by cryo-TEM imaging. The morphology of the nanorings exhibited the uniform circular conformation, and the sizes of the constructs are comparable to the DLS data (scale bar, 10 nm).

FIG. 14 shows the flow cytometry study of the transfer kinetics of the αEGFR-Fn3-Far CSANs from Raji cells to MDAMB-453-R cells. The CSAN-modified Raji cells were co-cultured with mKate-expressing EGFR-negative MDA-MB-453-R cells at a 6:4 ratio with rotation at 37° C. for 0-60 minutes, followed by flow cytometry analysis. No obvious cell-cell CSAN transfer was observed during the co-culture.

FIG. 15A-15B show the flow cytometry study of the cell-cell CSAN transfer at different temperatures. FIG. 15A shows the representative flow cytometry plots of the cell-cell CSAN transfer study at different temperatures (4° C. vs 37° C.). Raji cells were modified with αEGFR-Fn3-Far CSANs and co-cultured with A431-R cells at 4° C. or 37° C. for 30 minutes with rotation and the CSAN transfer was then detected and quantified by flow cytometry. The quantitative data of the flow cytometry analysis are presented in FIG. 15B which shows the percent cell populations of the cell co-cultures. A significantly higher level of CSAN transfer was observed when cells were co-cultured at 37° C., indicating the CSAN transfer is associated with the energy-dependent receptor internalization process of the receiver cells. For FIG. 15B, data are represented as mean values±SD (from n=3 independent experimental replicates). In some instances, small error bars are obscured by the symbols denoting the mean value.

FIGS. 16A-16B illustrate the flow cytometry study that showed the CSANs were able to transfer to receiver cells without mediating cell-cell interactions between sender cells and receiver cells. FIG. 16A show that Raji cells were stained with Hoechst dye as a marker and modified with different concentrations (0-2.5 μM) of fluorescein-labeled αEGFR-Fn3-Far CSANs, followed by co-culture with the mKate-expressing A431-R cells. The cells were analyzed by flow cytometry after the co-culture, and the cell-cell interactions were quantified by measuring the percentage of Hoechst⁺/mKate⁺ double-positive population, which indicates cell-cell clusters. The CSANs did not induce significant cell-cell interactions at low CSAN concentrations (<1 μM) for sender cell modifications. FIG. 16B shows that the CSAN transfer between sender cells and receiver cells was quantified by measuring the percentage of CSAN⁺/mKate⁺ double-positive populations. The CSANs were effectively transferred to the receiver cells even at low CSAN concentrations (<1 μM) for sender cell modifications.

FIGS. 17A-17B show the characterization of αCD133-scFv-DHFR² protein and the hybrid αCD133-scFv-Far CSANs. FIG. 17A shows that the αCD133-scFv-DHFR² protein was characterized by LC-MS (Orbitrap Elite). FIG. 17B shows that the hybrid αCD133-scFv-Far CSANs were formed by oligomerizing the αCD133-scFv-DHFR² protein and the DHFR²-Far protein at a 1:1 ratio. The hydrodynamic diameter of the hybrid αCD133-scFv-Far CSANs was measured to be 39.0±13.0 nm.

FIGS. 18A-18B show that the αCD133-scFv-DHFR2 CSANs were demonstrated to specifically bind to CD133+HT29-R cells. FIG. 18A shows that the HT29-R cells were confirmed to be CD133+ by flow cytometry, where the anti-CD133-PE antibody was used to detect the CD133, and over 90% of the HT29-R cells were shown to express CD133. FIG. 18B shows that the fluorescein-labeled αCD133-scFv-DHFR² CSANs were demonstrated to specifically bind to HT29-R cells by flow cytometry.

FIGS. 19A-19B show the LC-MS spectra of the protein-drug conjugates. The construction of (FIG. 19A) DHFR²-MMAE protein-drug conjugate and (FIG. 19B) αEGFR-Fn3-DHFR²-MMAE protein-drug conjugate was confirmed by LC-MS (Orbitrap Elite).

FIGS. 20A-20B show the characterization of the hybrid αEGFR-Fn3-Far-MMAE CSANs. FIG. 20A shows that the hybrid αEGFR-Fn3-Far-MMAE CSANs were formed by oligomerizing the αEGFR-Fn3-DHFR²-Far protein and the αEGFR-Fn3-DHFR²-MMAE protein. The hydrodynamic diameter of the hybrid αEGFR-Fn3-Far-MMAE CSANs was measured to be 40.0±4.7 nm. FIG. 20B shows that the hybrid αEGFR-Fn3-Far-MMAE CSANs were characterized by Cryo-TEM imaging. The morphology of the nanorings exhibited a uniform circular conformation, and the size of the constructs is comparable to the DLS data (scale bar, 10 nm).

FIG. 21 shows the 3-dimensional display of the internalization and localization of the fluorescein-labeled αEGFR-Fn3-DHFR²-MMAE CSANs in A431-R cells by fluorescent microscopy imaging. The A431-R cells were incubated with 0.5 μM of the fluorescein-labeled αEGFR-Fn3-DHFR²-MMAE CSANs at 37° C. for 1 h and then imaged by an Eclipse Ti-E Wide Field Deconvolution Inverted Microscope (Nikon Instruments, Inc.) using the Z-stack mode. The CSANs are shown in green and the nuclei are shown in red. The green punctate spots indicate the internalized CSANs and the yellow punctate spots indicate the localization of the CSANs in the red nuclei of the A431-R cells.

FIGS. 22A-22D show the LC-MS spectra of the αEGFR-Fn3-DHFR²-ssDNA protein-oligonucleotides conjugates. FIG. 22A shows that the construction of αEGFR-Fn3-DHFR²-ssDNA-AF488 protein-oligonucleotides conjugate was confirmed by LC-MS (Orbitrap Elite). FIG. 22B shows that the construction of αEGFR-Fn3-DHFR²-CTRLssDNA protein-oligonucleotides conjugate was confirmed by LC-MS (Orbitrap Elite). FIG. 22C shows that the construction of DHFR²-KDssDNA protein-oligonucleotides conjugate was confirmed by LC-MS (Orbitrap Elite). FIG. 22D shows that the construction of αEGFR-Fn3-DHFR²-KDssDNA protein-oligonucleotides conjugate was confirmed by LC-MS (Orbitrap Elite).

FIGS. 23A-23B show the characterization of the hybrid αEGFR-Fn3-Far-ssDNA CSANs. FIG. 23A shows that the hybrid αEGFR-Fn3-Far-ssDNAAF488 CSANs were formed by oligomerizing the αEGFR-Fn3-DHFR²-Far protein and the αEGFR-Fn3-DHFR²-ssDNAAF488 protein. The hydrodynamic diameter of the hybrid αEGFR-Fn3-Far-ssDNA-AF488 CSANs was measured to be 39.0±9.1 nm. FIG. 23B shows that the hybrid αEGFR-Fn3-Far-ssDNA-AF488 CSANs were characterized by Cryo-TEM imaging. The morphology of the nanorings exhibited a uniform circular conformation, and the size of the constructs is comparable to the DLS data (scale bar, 10 nm).

FIG. 24 shows the percentage of MSCs positive for CSANs via flow cytometry demonstrating no significant difference before and after shearing under two separate conditions.

FIG. 25 shows the MFI of CSANs on MSCs before and after shearing.

FIG. 26 shows the transfer of 500 nM CSANs from MSCs to A431 cells over 24 hrs.

### DETAILED DESCRIPTION

It is herein reported that lapidated chemically self-assembled antibody nanorings (CSAN), such as f-CSAN, on sender cell membranes undergo efficient CSAN-assisted cell-cell cargo transfer (C4T). For example, by incorporating azide-functionalized prenyl (e.g., farnesyl) analogs as bioconjugation handles, both small molecule drug and macromolecule cargo such as antisense oligonucleotide were able to undergo C4T transfer. Thus, the C4T approach was shown herein to be a versatile approach for demonstrating cell-cell interactions as well as engineering cell-cell communication. Additionally, C4T approach also provides methods for cell-based cargo delivery (e.g., cell-based drug delivery or detectable agent delivery).

Accordingly, certain embodiments of the invention provide a fusion protein described herein.

Certain embodiments of the invention provide a conjugate described herein.

Certain embodiments of the invention provide a chemically self-assembled antibody nanorings (CSAN) described herein.

Certain embodiments of the invention provide a CSAN modified cell described herein.

Certain embodiments of the invention provide a method of using a fusion protein, a conjugate, a CSAN, or a CSAN modified cell described herein, such as a method for cell-based drug delivery.

### Fusion Proteins

The term “fusion protein” as used herein refers to a recombinant protein comprising two or more domains as described herein, including but not limited to, targeting domain, DHFR² domain, and/or prenyltransferase substrate domain. In certain embodiments, a fusion protein described herein could participate in the assembly of a chemically self-assembled antibody nanorings (CSAN) in the presence of bisMTX compound. In certain embodiments, a fusion protein described herein could be enzymatically lapidated into a conjugate as described herein (e.g., a lapidated fusion protein).

The term “targeting domain” as used herein refers to a peptide or polypeptide (e.g., protein) as a domain or subunit of the fusion protein described herein, which has affinity for a target (e.g., a cell membrane anchored protein). In certain embodiments, the targeting domain may have affinity for a cell surface protein, such as a cell membrane anchored protein. In certain embodiments, the polypeptide may be an antibody mimetic such as affibody or human tenth type III fibronectin scaffold (Fn3). In certain embodiments, the polypeptide may be a nanobody, or an antigen binding fragment of an antibody. In certain embodiments, the peptide comprises Arg-Gly-Asp (RGD) sequence or motif.

In certain embodiments, the target is a cell surface protein. In certain embodiments, the target is a cell surface protein expressed or overexpressed by a cancer cell (e.g., a malignant cell, or a cancer stem-like cells (CSC) that is also referred to as cancer stem cell). In certain embodiments, the target is human HER2, EGFR, EpCAM, or CD133. In certain embodiments, the target is human integrin αvβ3.

In certain embodiments, the targeting domain comprises an affibody. In certain embodiments, the targeting domain comprises human tenth type III fibronectin scaffold (Fn3).

In certain embodiments, the targeting domain does not comprise disulfide bond.

In certain embodiments, the targeting domain comprises disulfide bond(s).

In certain embodiments, the targeting domain comprises a nanobody. In certain embodiments, the targeting domain comprises a scFv.

In certain embodiments, the targeting domain is an anti-EGFR Fn3, or anti-EpCAM Fn3.

In certain embodiments, the targeting domain is an anti-HER2 affibody.

In certain embodiments, the targeting domain is an anti-CD”133 scFv.

In certain embodiments, the targeting domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to the anti-HER2 affibody sequence described herein (SEQ ID NO:8). In certain embodiments, the targeting domain comprises an amino acid sequence having at least 85% sequence identify to SEQ ID NO:8. In certain embodiments, the targeting domain comprises an amino acid sequence having at least 90% sequence identify to SEQ ID NO:8. In certain embodiments, the targeting domain comprises an amino acid sequence having at least 95% sequence identify to SEQ ID NO:8. In certain embodiments, the targeting domain comprises the amino acid sequence of SEQ ID NO:8. In certain embodiments, the targeting domain consists of the amino acid sequence of SEQ ID NO:8.

In certain embodiments, the targeting domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to the anti-EpCAM Fn3 sequence described herein (SEQ ID NO:9). In certain embodiments, the targeting domain comprises an amino acid sequence having at least 85% sequence identify to SEQ ID NO:9. In certain embodiments, the targeting domain comprises an amino acid sequence having at least 90% sequence identify to SEQ ID NO:9. In certain embodiments, the targeting domain comprises an amino acid sequence having at least 95% sequence identify to SEQ ID NO:9. In certain embodiments, the targeting domain comprises the amino acid sequence of SEQ ID NO:9. In certain embodiments, the targeting domain consists of the amino acid sequence of SEQ ID NO:9.

In certain embodiments, the targeting domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to the anti-EGFR Fn3 sequence described herein (SEQ ID NO:10). In certain embodiments, the targeting domain comprises an amino acid sequence having at least 85% sequence identify to SEQ ID NO:10. In certain embodiments, the targeting domain comprises an amino acid sequence having at least 90% sequence identify to SEQ ID NO:10. In certain embodiments, the targeting domain comprises an amino acid sequence having at least 95% sequence identify to SEQ ID NO:10. In certain embodiments, the targeting domain comprises the amino acid sequence of SEQ ID NO:10. In certain embodiments, the targeting domain consists of the amino acid sequence of SEQ ID NO:10.

In certain embodiments, the targeting domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to the anti-CD133 scFv sequence described herein (SEQ ID NO:11). In certain embodiments, the targeting domain comprises an amino acid sequence having at least 85% sequence identify to SEQ ID NO:11. In certain embodiments, the targeting domain comprises an amino acid sequence having at least 90% sequence identify to SEQ ID NO:11. In certain embodiments, the targeting domain comprises an amino acid sequence having at least 95% sequence identify to SEQ ID NO:11. In certain embodiments, the targeting domain comprises the amino acid sequence of SEQ ID NO:11. In certain embodiments, the targeting domain consists of the amino acid sequence of SEQ ID NO:11.

The term “dihydrofolate reductase (DHFR)” refers to a polypeptide as a domain or subunit of the fusion protein described herein, which could bind a BisMTX compound and facilitate the oligomerization of fusion proteins into a CSAN as described herein. In certain embodiments, the fusion protein described herein comprise a first DHFR and a second DHFR (also referred to as DHFR²).

In certain embodiments, the DHFR domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to any of SEQ ID NOs:1-4. In certain embodiments, the DHFR domain comprises an amino acid sequence having at least 85% sequence identify to any of SEQ ID NOs:1-4. In certain embodiments, the DHFR domain comprises an amino acid sequence having at least 90% sequence identify to any of SEQ ID NOs:1-4. In certain embodiments, the DHFR domain comprises an amino acid sequence having at least 95% sequence identify to any of SEQ ID NOs:1-4. In certain embodiments, the DHFR domain comprises the amino acid sequence of any of SEQ ID NOs:1-4. In certain embodiments, the DHFR domain consists of the amino acid sequence of any of SEQ ID NOs:1-4.

In certain embodiments, the DHFR² domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to any of SEQ ID NOs:5-7. In certain embodiments, the DHFR² domain comprises an amino acid sequence having at least 85% sequence identify to any of SEQ ID NOs:5-7. In certain embodiments, the DHFR² domain comprises an amino acid sequence having at least 90% sequence identify to any of SEQ ID NOs:5-7. In certain embodiments, the DHFR² domain comprises an amino acid sequence having at least 95% sequence identify to any of SEQ ID NOs:5-7. In certain embodiments, the DHFR² domain comprises the amino acid sequence of any of SEQ ID NOs:5-7. In certain embodiments, the DHFR² domain consists of the amino acid sequence of any of SEQ ID NOs:5-7.

The term “prenyltransferase substrate domain” refers to a polypeptide as a domain or subunit of the fusion protein described herein, which can be recognized by a prenyltransferase (e.g., farnesyltransferase or geranylgeranyltransferase) for prenylation process that could conjugate a prenyl lipid such as C10 (geranyl), C15 (farnesyl) or C20 (geranylgeranyl) lipid onto the fusion protein. This domain may be modified by prenyltransferase into a prenylated (e.g., a geranylated, farnesylated, or geranylgeranylated) polypeptide. For example, the domain may comprise CAAX-motif (also referred to as CAAX box), wherein C is Cys; A is each independently an aliphatic amino acid, such as Ala, Val, Ile, or Leu; and X can be a variety of amino acids, such as Ala, Gln, Ser, Met, Phe, or Leu. In certain embodiments, X is Ala, Gln, Ser, Met, or Phe. In certain embodiments, X is Leu, Met, or Phe. Prenyltransferase substrate domain comprising CAAX motif is described herein and known in the art, for example, in documents: K. T. Lane, et al., Lipid Res., 2006, 47, 681-699; T. Scott Reid, et al., J. Mol. Biol., 2004, 343, 417-433; T. C. Turek-Etienne, et al., Biochemistry, 2003, 42, 3716-3724; Y. Wang, et al., ACS Chem. Biol., 2014, 9(8), 1726-1735; J. L. Hougland, et al., J. Mol. Biol., 2010, 395, 176-190; which are incorporated by reference herein.

In certain embodiments, the prenyltransferase substrate domain comprises a CAAX-motif wherein A is each independently Val or Ile. In certain embodiments, the prenyltransferase substrate domain comprises a CAAX-motif wherein X is Ala.

In certain embodiments, the prenyltransferase substrate domain comprises an amino acid sequence of CVIA (Cys-Val-Ile-Ala).

In certain embodiments, the prenyltransferase substrate domain comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to a CVIA sequence described herein (SEQ ID NO:12). In certain embodiments, the prenyltransferase substrate domain comprises an amino acid sequence having at least 85% sequence identify to SEQ ID NO:12. In certain embodiments, the prenyltransferase substrate domain comprises an amino acid sequence having at least 90% sequence identify to SEQ ID NO:12. In certain embodiments, the prenyltransferase substrate domain comprises an amino acid sequence having at least 95% sequence identify to SEQ ID NO:12. In certain embodiments, the prenyltransferase substrate domain comprises the amino acid sequence of SEQ ID NO:12. certain embodiments, the prenyltransferase substrate domain consists of the amino acid sequence of SEQ ID NO:12.

In certain embodiments, the fusion protein described herein comprise a DHFR² domain and a prenyltransferase substrate domain. In certain embodiments, the fusion protein described herein comprise a targeting domain and a DHFR² domain. In certain embodiments, the fusion protein described herein comprise a targeting domain, a DHFR² domain, and a prenyltransferase substrate domain.

In certain embodiments, the fusion protein described herein comprise a targeting domain, a first DHFR, a second DHFR and a prenyltransferase substrate domain operably linked together from N-terminal to C-terminal. In certain embodiments, the fusion protein described herein comprise a targeting domain, a first DHFR, a second DHFR and a prenyltransferase substrate domain operably linked together via one or more linker.

In certain embodiments, the fusion protein comprises one or more optional linker between adjacent domains. For example, each domain may be operably linked via one or more linkers to form the fusion protein. In certain embodiments, the targeting domain is operably linked to the first DHFR via a linker. In certain embodiments, the first DHFR is operably linked to the second DHFR via a linker. In certain embodiments, the second DHFR is operably linked to the prenyltransferase substrate domain via a linker.

Each optional linker between domains of the fusion protein is independently a peptide or polypeptide linkers. In certain embodiments, the linker is a flexible peptide or polypeptide linker. In certain embodiments, the linker is a glycine, glycine-serine, or glycine rich linker (e.g., more than 60% of the linker sequence is glycine). In certain embodiments, the linker comprises G, GG, GSG, GGS, GGGS, GGGGS, GGSGGS, GSSGSS, or combination thereof. In certain embodiments, the linker is a glycine or glycine-serine linker as described in Table A1.

In certain embodiments, the fusion protein comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to the αHER2-afb-DHFR2-CVIA sequence described herein (SEQ ID NO:14). In certain embodiments, the fusion protein comprises an amino acid sequence having at least 85% sequence identify to SEQ ID NO:14. In certain embodiments, the fusion protein comprises an amino acid sequence having at least 90% sequence identify to SEQ ID NO:14. In certain embodiments, the fusion protein comprises an amino acid sequence having at least 95% sequence identify to SEQ ID NO:14. In certain embodiments, the fusion protein comprises the amino acid sequence of SEQ ID NO:14. In certain embodiments, the fusion protein consists of the amino acid sequence of SEQ ID NO:14.

In certain embodiments, the fusion protein comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to the αEpCam-Fn3-DHFR2-CVIA sequence described herein (SEQ ID NO:15). In certain embodiments, the fusion protein comprises an amino acid sequence having at least 85% sequence identify to SEQ ID NO:15. In certain embodiments, the fusion protein comprises an amino acid sequence having at least 90% sequence identify to SEQ ID NO:15. In certain embodiments, the fusion protein comprises an amino acid sequence having at least 95% sequence identify to SEQ ID NO:15. In certain embodiments, the fusion protein comprises the amino acid sequence of SEQ ID NO:15. In certain embodiments, the fusion protein consists of the amino acid sequence of SEQ ID NO:15.

In certain embodiments, the fusion protein comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to the αEGFR-Fn3-DHFR2-CVIA sequence described herein (SEQ ID NO:16). In certain embodiments, the fusion protein comprises an amino acid sequence having at least 85% sequence identify to SEQ ID NO:16. In certain embodiments, the fusion protein comprises an amino acid sequence having at least 90% sequence identify to SEQ ID NO:16. In certain embodiments, the fusion protein comprises an amino acid sequence having at least 95% sequence identify to SEQ ID NO:16. In certain embodiments, the fusion protein comprises the amino acid sequence of SEQ ID NO:16. In certain embodiments, the fusion protein consists of the amino acid sequence of SEQ ID NO:16.

In certain embodiments, the fusion protein comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to the αCD133-scFv-DHFR² sequence described herein (SEQ ID NO:17). In certain embodiments, the fusion protein comprises an amino acid sequence having at least 85% sequence identify to SEQ ID NO:17. In certain embodiments, the fusion protein comprises an amino acid sequence having at least 90% sequence identify to SEQ ID NO:17. In certain embodiments, the fusion protein comprises an amino acid sequence having at least 95% sequence identify to SEQ ID NO:17. In certain embodiments, the fusion protein comprises the amino acid sequence of SEQ ID NO:17. In certain embodiments, the fusion protein consists of the amino acid sequence of SEQ ID NO:17.

In certain embodiments, the fusion protein comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identify to the DHFR²-CVIA sequence (SEQ ID NO:18) described herein. In certain embodiments, the fusion protein comprises an amino acid sequence having at least 85% sequence identify to SEQ ID NO:18. In certain embodiments, the fusion protein comprises an amino acid sequence having at least 90% sequence identify to SEQ ID NO:18. In certain embodiments, the fusion protein comprises an amino acid sequence having at least 95% sequence identify to SEQ ID NO:18. In certain embodiments, the fusion protein comprises the amino acid sequence of SEQ ID NO:18. In certain embodiments, the fusion protein consists of the amino acid sequence of SEQ ID NO:18.

In certain embodiments, a detectable agent such as a fluorescent dye may be directly linked to the fusion protein as described herein (e.g., a fusion protein non-specifically labeled with fluorescein).

TABLE Al
Exemplary sequences of fusion proteins and domains

| SEQ | | |
|---|---|---|
| ID | | Domain or |
| NO | Sequence | Construct |
| 1 | ISLIAALAVDRVIGMENAMPWNLPADLAWFKRNTLNKPVIMGRHTWESIGRPLPGRKN | an exemplary |
| | IILSSQPGTDDRVTWVKSVDEAIAAAGDVPEIMVIGGGRVYEQFLPKAQKLYLTHIDAEV | DHFR |
| | EGDTHFPDYEPDDWESVFSEFHDADAQNSHSYSFEILERR | sequence |
| 2 | GISLIAALAVDRVIGMENAMPWNLPADLAWFKRNTLNKPVIMGRHTWESIGRPLPGRK | an exemplary |
| | NIILSSQPGTDDRVTWVKSVDEAIAAAGDVPEIMVIGGGRVYEQFLPKAQKLYLTHIDAE | DHFR |
| | VEGDTHFPDYEPDDWESVFSEFHDADAQNSHSYSFEILERR | sequence |
| 3 | LISLIAALAVDRVIGMENAMPWNLPADLAWFKRNTLNKPVIMGRHTWESIGRPLPGRK | an exemplary |
| | NIILSSQPGTDDRVTWVKSVDEAIAAAGDVPEIMVIGGGRVYEQFLPKAQKLYLTHIDAE | DHFR |
| | VEGDTHFPDYEPDDWESVFSEFHDADAQNSHSYSFEILERR | sequence |
| 4 | MISLIAALAVDRVIGMENAMPWNLPADLAWFKRNTLNKPVIMGRHTWESIGRPLPGR | an exemplary |
| | KNIILSSQPGTDDRVTWVKSVDEAIAAAGDVPEIMVIGGGRVYEQFLPKAQKLYLTHIDA | DHFR |
| | EVEGDTHFPDYEPDDWESVFSEFHDADAQNSHSYSFEILERR | sequence |
| 5 | LISLIAALAVDRVIGMENAMPWNLPADLAWFKRNTLNKPVIMGRHTWESIGRPLPGRK | an exemplary |
| | NIILSSQPGTDDRVTWVKSVDEAIAAAGDVPEIMVIGGGRVYEQFLPKAQKLYLTHIDAE | DHFR₂ |
| | VEGDTHFPDYEPDDWESVFSEFHDADAQNSHSYSFEILERRGLISLIAALAVDRVIGMEN | sequence |
| | AMPWNLPADLAWFKRNTLNKPVIMGRHTWESIGRPLPGRKNIILSSQPGTDDRVTWV | (Glycine linker |
| | KSVDEAIAAAGDVPEIMVIGGGRVYEQFLPKAQKLYLTHIDAEVEGDTHFPDYEPDDWE | is underlined) |
| | SVFSEFHDADAQNSHSYSFEILERR | |
| 6 | GISLIAALAVDRVIGMENAMPWNLPADLAWFKRNTLNKPVIMGRHTWESIGRPLPGRK | an exemplary |
| | NIILSSQPGTDDRVTWVKSVDEAIAAAGDVPEIMVIGGGRVYEQFLPKAQKLYLTHIDAE | DHFR₂ |
| | VEGDTHFPDYEPDDWESVFSEFHDADAQNSHSYSFEILERRGGISLIAALAVDRVIGMEN | sequence |
| | AMPWNLPADLAWFKRNTLNKPVIMGRHTWESIGRPLPGRKNIILSSQPGTDDRVTWV | (Glycine linker |
| | KSVDEAIAAAGDVPEIMVIGGGRVYEQFLPKAQKLYLTHIDAEVEGDTHFPDYEPDDWE | is underlined) |
| | SVFSEFHDADAQNSHSYSFEILERR | |
| 7 | MISLIAALAVDRVIGMENAMPWNLPADLAWFKRNTLNKPVIMGRHTWESIGRPLPGR | an exemplary |
| | KNIILSSQPGTDDRVTWVKSVDEAIAAAGDVPEIMVIGGGRVYEQFLPKAQKLYLTHIDA | DHFR₂ |
| | EVEGDTHFPDYEPDDWESVFSEFHDADAQNSHSYSFEILERRGMISLIAALAVDRVIGME | sequence |
| | NAMPWNLPADLAWFKRNTLNKPVIMGRHTWESIGRPLPGRKNIILSSQPGTDDRVTW | (Glycine linker |
| | VKSVDEAIAAAGDVPEIMVIGGGRVYEQFLPKAQKLYLTHIDAEVEGDTHFPDYEPDDW | is underlined) |
| | ESVFSEFHDADAQNSHSYSFEILERR | |
| 8 | EAKYAKEMRNAYWEIALLPNLTNQQKRAFIRKLYDDPSQSSELLSEAKKLNDSQAPK | an exemplary |
| | | anti-HER2 |
| | | affibody |
| | | sequence |
| 9 | SSDSPRNLEVTNATPNSLTISWDNSNYASYYRITYGETGGNSPSQELTVPGSTYNATISGL | an exemplary |
| | KPGQDYIITVYAVTYRDNYSYSNLISINYRSEIDKPSQ | anti-EpCAM |
| | | Fn3 sequence |
| | | (C5 |
| 10 | VSDVPRDLEVVAATPTSLLISWDSGRGSYQYRITYGETGGNSPVQEFTVPGPVHTATISGL | an exemplary |
| | KPGVDYTITVYAVTDHKPHADGPHTYHESPISINYRT | anti-EGFR Fn3 |
| | | sequence (E1) |
| 11 | MDIVLSQSPAIMSASPGEKVTISCSASSSVSYMYWYQQKPGSSPKPWIYRTSNLASGVPA | an exemplary |
| | RFSGSGSGTSYSLTISSMEAEDAATYYCQQYHSYPPTFGAGTKLELKSSGGGGSGGGGG | anti-cd133 |
| | GSSRSSLEVKLVESGPELKKPGETVKISCKASGYTFTDYSMHWVNQAPGKGLKWMGWI | scFv sequence |
| | NTETGEPSYADDFKGRFAFSLETSASTAYLQINNLKNEDTATYFCATDYGDYFDYWGQG | (GS linker is |
| | TTLTVSS | underlined) |
| 12 | KKKKKKTCVIA | An exemplary |
| | | CVIA tag |
| | | sequence |
| 13 | HHHHHH | An exemplary |
| | | HIS tag |
| | | sequence |
| 14 | MAEAKYAKEMRNAYWEIALLPNLTNQQKRAFIRKLYDDPSQSSELLSEAKKLNDSQAPK | anti-Her2-afb- |
| | GGGSGGGSGGGSGGLISLIAALAVDRVIGMENAMPWNLPADLAWFKRNTLNKPVIM | 1DD-CVIA |
| | GRHTWESIGRPLPGRKNIILSSQPGTDDRVTWVKSVDEAIAAAGDVPEIMVIGGGRVY | (anti-Her2 |
| | EQFLPKAQKLYLTHIDAEVEGDTHFPDYEPDDWESVFSEFHDADAQNSHSYSFEILERR | affibody |
| | GLISLIAALAVDRVIGMENAMPWNLPADLAWFKRNTLNKPVIMGRHTWESIGRPLPG | sequence is |
| | RKNIILSSQPGTDDRVTWVKSVDEAIAAAGDVPEIMVIGGGRVYEQFLPKAQKLYLTHI | italicized; |
| | DAEVEGDTHFPDYEPDDWESVFSEFHDADAQNSHSYSFEILERRGGSGGSGGELDYKD | DHFR₂ is |
| | DDDKGGSGGSGGKKKKKKTCVIA | bolded; and |
| | | CVIA tag |
| | | sequence is |
| | | underlined) |
| 15 | MDYKDDDDKASSSDSPRNLEVTNATPNSLTISWDNSNYASYYRITYGETGGNSPSQELTV | anti-EpCAM- |
| | PGSTYNATISGLKPGQDYIITVYAVTYRDNYSYSNLISINYRSEIDKPSQGSGGSGGGGSGG | C5-Fn-1DD- |
| | GGSGGGGSGGGGSGGGGSGGGGSGGGGSGGGGSGGELGGISLIAALAVDRVIGMEN | CVIA |
| | AMPWNLPADLAWFKRNTLNKPVIMGRHTWESIGRPLPGRKNIILSSQPGTDDRVTW | (anti-EpCAM- |
| | VKSVDEAIAAAGDVPEIMVIGGGRVYEQFLPKAQKLYLTHIDAEVEGDTHFPDYEPDD | C5-Fn |
| | WESVFSEFHDADAQNSHSYSFEILERRGGISLIAALAVDRVIGMENAMPWNLPADLA | sequence is |
| | WFKRNTLNKPVIMGRHTWESIGRPLPGRKNIILSSQPGTDDRVTWVKSVDEAIAAAG | italicized; |
| | DVPEIMVIGGGRVYEQFLPKAQKLYLTHIDAEVEGDTHFPDYEPDDWESVFSEFHDAD | DHFR₂ is |
| | AQNSHSYSFEILERRGGSGGGSGGHHHHHHGGSGGSGGKKKKKKTCVIA | bolded; and |
| | | CVIA tag |
| | | sequence is |
| | | underlined) |
| 16 | MGVSDVPRDLEVVAATPTSLLISWDSGRGSYQYRITYGETGGNSPVQEFTVPGPVHTATI | anti-EGFR-E1- |
| | SGLKPGVDYTITVYAVTDHKPHADGPHTYHESPISINYRTDIDRPSQGGSLISLIAALAVDR | Fn-1DD-CVIA |
| | VIGMENAMPWNLPADLAWFKRNTLNKPVIMGRHTWESIGRPLPGRKNIILSSQPGT | (anti-EpCAM- |
| | DDRVTWVKSVDEAIAAAGDVPEIMVIGGGRVYEQFLPKAQKLYLTHIDAEVEGDTHF | C5-Fn |
| | PDYEPDDWESVFSEFHDADAQNSHSYSFEILERRGLISLIAALAVDRVIGMENAMPW | sequence is |
| | NLPADLAWFKRNTLNKPVIMGRHTWESIGRPLPGRKNIILSSQPGTDDRVTWVKSVD | italicized; |
| | EAIAAAGDVPEIMVIGGGRVYEQFLPKAQKLYLTHIDAEVEGDTHFPDYEPDDWESVF | DHFR₂ is |
| | SEFHDADAQNSHSYSFEILERRGGSGGSGGELDYKDDDDKGGSGGSGGKKKKKKTCVIA | bolded; and |
| | | CVIA tag |
| | | sequence is |
| | | underlined) |
| 17 | MDIVLSQSPAIMSASPGEKVTISCSASSSVSYMYWYQQKPGSSPKPWIYRTSNLASGVPA | anti-CD133- |
| | RFSGSGSGTSYSLTISSMEAEDAATYYCQQYHSYPPTFGAGTKLELKSSGGGGSGGGGGG | 1DD |
| | SSRSSLEVKLVESGPELKKPGETVKISCKASGYTFTDYSMHWVNQAPGKGLKWMGWIN | (anti-CD133 |
| | TETGEPSYADDFKGRFAFSLETSASTAYLQINNLKNEDTATYFCATDYGDYFDYWGQGTT | scFv sequence |
| | LTVSSELGGSGGGSGGGSGGMISLIAALAVDRVIGMENAMPWNLPADLAWFKRNTL | is italicized; |
| | NKPVIMGRHTWESIGRPLPGRKNIILSSQPGTDDRVTWVKSVDEAIAAAGDVPEIMVI | and |
| | GGGRVYEQFLPKAQKLYLTHIDAEVEGDTHFPDYEPDDWESVFSEFHDADAQNSHSY | DHFR₂ is |
| | SFEILERRGMISLIAALAVDRVIGMENAMPWNLPADLAWFKRNTLNKPVIMGRHTW | bolded) |
| | ESIGRPLPGRKNIILSSQPGTDDRVTWVKSVDEAIAAAGDVPEIMVIGGGRVYEQFLPK | |
| | AQKLYLTHIDAEVEGDTHFPDYEPDDWESVFSEFHDADAQNSHSYSFEILERRGGSGG | |
| | GSGGGSGGDYKDDDDK | |
| 18 | GISLIAALAVDRVIGMENAMPWNLPADLAWFKRNTLNKPVIMGRHTWESIGRPLPG | an exemplary |
| | RKNIILSSQPGTDDRVTWVKSVDEAIAAAGDVPEIMVIGGGRVYEQFLPKAQKLYLTHI | DHFR₂-CVIA |
| | DAEVEGDTHFPDYEPDDWESVFSEFHDADAQNSHSYSFEILERRGGISLIAALAVDRVI | sequence |
| | GMENAMPWNLPADLAWFKRNTLNKPVIMGRHTWESIGRPLPGRKNIILSSQPGTDD | (DHFR₂ is |
| | RVTWVKSVDEAIAAAGDVPEIMVIGGGRVYEQFLPKAQKLYLTHIDAEVEGDTHFPDY | bolded; and |
| | EPDDWESVFSEFHDADAQNSHSYSFEILERRGGSGGSGGDYKDDDDKGGSGGSGGKK | CVIA tag |
| | KKKKTCVIA | sequence is |
| | | underlined) |
| 19 | GGCATCAGTCTGATTGCGGCGTTAGCGGTAGATCGCGTTATCGGCATGGAAAACGC | Gene |
| | CATGCCGTGGAACCTGCCTGCCGATCTCGCCTGGTTTAAACGCAACACCTTAAATAAA | sequence |
| | CCCGTGATTATGGGCCGCCATACCTGGGAATCAATCGGTCGTCCGTTGCCAGGACGC | encoding |
| | AAAAATATTATCCTCAGCAGTCAACCGGGTACGGACGATCGCGTAACGTGGGTGAA | DHFR²-CVIA |
| | GTCGGTGGATGAAGCCATCGCGGCGGCTGGTGACGTACCAGAAATCATGGTGATTG | |
| | GCGGCGGTCGCGTTTATGAACAGTTCTTGCCAAAAGCGCAAAAACTGTATCTGACGC | |
| | ATATCGACGCAGAAGTGGAAGGCGACACCCATTTCCCGGATTACGAGCCGGATGAC | |
| | TGGGAATCGGTATTCAGTGAATTCCACGATGCTGATGCGCAGAACTCTCACAGCTAT | |
| | AGCTTTGAGATTCTGGAGCGGCGGGGCGGCATTAGCCTTATTGCCGCCTTAGCGGTT | |
| | GATCGCGTGATCGGAATGGAGAACGCAATGCCCTGGAATCTTCCGGCAGACCTTGCC | |
| | TGGTTCAAACGCAACACTTTAAACAAGCCTGTCATTATGGGCCGTCACACATGGGAG | |
| | TCAATTGGTCGTCCCCTGCCTGGGCGCAAAAATATCATCTTGTCCTCGCAGCCTGGGA | |
| | CAGATGATCGCGTTACATGGGTGAAGTCCGTAGACGAAGCGATTGCCGCTGCCGGC | |
| | GATGTGCCCGAGATTATGGTAATCGGGGGAGGGCGTGTTTACGAACAATTTCTGCCC | |
| | AAAGCTCAGAAATTATACCTGACGCACATCGACGCGGAGGTCGAAGGTGACACACA | |
| | CTTTCCAGATTATGAGCCTGATGATTGGGAATCCGTTTTCTCAGAATTTCATGACGCG | |
| | GATGCTCAAAACTCGCACTCGTACTCTTTTGAAATTTTAGAGCGCCGTGGCGGATCTG | |
| | GAGGAAGTGGCGGTGACTACAAAGACGACGATGATAAGGGCGGCTCAGGTGGTTC | |
| | CGGTGGCAAAAAGAAAAAGAAAAAGACCTGTGTCATCGCCTAGTGA | |
| 20 | ATGGACTACAAAGACGACGATGATAAGGGCGGATCTGGAGGAAGTGGCGGTATGG | Gene |
| | GTGTCTCTGACGTCCCGCGTGACCTGGAGGTTGTTGCAGCGACCCCAACTAGCCTTC | sequence |
| | TTATCAGCTGGGATAGCGGTCGTGGTTCTTATCAATACTACCGGATCACTTACGGAG | encoding |
| | AAACAGGAGGAAATAGCCCTGTTCAGGAGTTCACTGTGCCTGGTCCTGTACACACTG | aEGFR-Fn3- |
| | CTACCATCAGCGGCCTTAAACCTGGAGTAGATTATACCATCACTGTGTATGCTGTCAC | DHFR²-CVIA |
| | TGACCATAAGCCTCATGCTGATGGACCTCACACTTATCATGAATCTCCAATTTCCATCA | |
| | ATTACCGTACAGATATTGATCGTCCTTCTCAAGGTGGTAGTGGCATCAGTCTGATTGC | |
| | GGCGTTAGCGGTAGATCGCGTTATCGGCATGGAAAACGCCATGCCGTGGAACCTGC | |
| | CTGCCGATCTCGCCTGGTTTAAACGCAACACCTTAAATAAACCCGTGATTATGGGCCG | |
| | CCATACCTGGGAATCAATCGGTCGTCCGTTGCCAGGACGCAAAAATATTATCCTCAG | |
| | CAGTCAACCGGGTACGGACGATCGCGTAACGTGGGTGAAGTCGGTGGATGAAGCCA | |
| | TCGCGGCGGCTGGTGACGTACCAGAAATCATGGTGATTGGCGGCGGTCGCGTTTAT | |
| | GAACAGTTCTTGCCAAAAGCGCAAAAACTGTATCTGACGCATATCGACGCAGAAGTG | |
| | GAAGGCGACACCCATTTCCCGGATTACGAGCCGGATGACTGGGAATCGGTATTCAGT | |
| | GAATTCCACGATGCTGATGCGCAGAACTCTCACAGCTATAGCTTTGAGATTCTGGAG | |
| | CGGCGGGGCGGCATTAGCCTTATTGCCGCCTTAGCGGTTGATCGCGTGATCGGAAT | |
| | GGAGAACGCAATGCCCTGGAATCTTCCGGCAGACCTTGCCTGGTTCAAACGCAACAC | |
| | TTTAAACAAGCCTGTCATTATGGGCCGTCACACATGGGAGTCAATTGGTCGTCCCCTG | |
| | CCTGGGCGCAAAAATATCATCTTGTCCTCGCAGCCTGGGACAGATGATCGCGTTACA | |
| | TGGGTGAAGTCCGTAGACGAAGCGATTGCCGCTGCCGGCGATGTGCCCGAGATTAT | |
| | GGTAATCGGGGGAGGGCGTGTTTACGAACAATTTCTGCCCAAAGCTCAGAAATTATA | |
| | CCTGACGCACATCGACGCGGAGGTCGAAGGTGACACACACTTTCCAGATTATGAGCC | |
| | TGATGATTGGGAATCCGTTTTCTCAGAATTTCATGACGCGGATGCTCAAAACTCGCAC | |
| | TCGTACTCTTTTGAAATTTTAGAGCGCCGTGGCGGCTCAGGTGGTTCCGGTGGCCAT | |
| | CATCATCATCATCACGGCGGCTCAAAAAAGAAAAAGAAAAAGACCTGTGTCATCGCC | |
| | TAGTGA | |
| 21 | ATGGACTACAAAGACGACGATGATAAGGGCGGATCTGGAGGAAGTGGCGGTATGG | Gene |
| | CGGAGGCGAAGTACGCGAAAGAAATGCGTAACGCGTATTGGGAGATCGCGCTGCT | sequence |
| | GCCGAACCTGACCAACCAGCAAAAGCGTGCGTTCATTCGTAAACTGTACGACGATCC | encoding |
| | GAGCCAGAGCAGCGAGCTGCTGAGCGAAGCGAAGAAACTGAACGACAGCCAAGCG | aHER2-afb- |
| | CCGAAGGGCGGCGGTAGTGGCGGTGGCAGCGGTGGCGGTAGCGGCGGTGGCATC | DHFR²-CVIA |
| | AGTCTGATTGCGGCGTTAGCGGTAGATCGCGTTATCGGCATGGAAAACGCCATGCC | |
| | GTGGAACCTGCCTGCCGATCTCGCCTGGTTTAAACGCAACACCTTAAATAAACCCGT | |
| | GATTATGGGCCGCCATACCTGGGAATCAATCGGTCGTCCGTTGCCAGGACGCAAAAA | |
| | TATTATCCTCAGCAGTCAACCGGGTACGGACGATCGCGTAACGTGGGTGAAGTCGGT | |
| | GGATGAAGCCATCGCGGCGGCTGGTGACGTACCAGAAATCATGGTGATTGGCGGCG | |
| | GTCGCGTTTATGAACAGTTCTTGCCAAAAGCGCAAAAACTGTATCTGACGCATATCG | |
| | ACGCAGAAGTGGAAGGCGACACCCATTTCCCGGATTACGAGCCGGATGACTGGGAA | |
| | TCGGTATTCAGTGAATTCCACGATGCTGATGCGCAGAACTCTCACAGCTATAGCTTTG | |
| | AGATTCTGGAGCGGCGGGGCGGCATTAGCCTTATTGCCGCCTTAGCGGTTGATCGC | |
| | GTGATCGGAATGGAGAACGCAATGCCCTGGAATCTTCCGGCAGACCTTGCCTGGTTC | |
| | AAACGCAACACTTTAAACAAGCCTGTCATTATGGGCCGTCACACATGGGAGTCAATT | |
| | GGTCGTCCCCTGCCTGGGCGCAAAAATATCATCTTGTCCTCGCAGCCTGGGACAGAT | |
| | GATCGCGTTACATGGGTGAAGTCCGTAGACGAAGCGATTGCCGCTGCCGGCGATGT | |
| | GCCCGAGATTATGGTAATCGGGGGAGGGCGTGTTTACGAACAATTTCTGCCCAAAG | |
| | CTCAGAAATTATACCTGACGCACATCGACGCGGAGGTCGAAGGTGACACACACTTTC | |
| | CAGATTATGAGCCTGATGATTGGGAATCCGTTTTCTCAGAATTTCATGACGCGGATG | |
| | CTCAAAACTCGCACTCGTACTCTTTTGAAATTTTAGAGCGCCGTGGCGGCTCAGGTG | |
| | GTTCCGGTGGCAAAAAGAAAAAGAAAAAGACCTGTGTCATCGCCTAGTGA | |
| 22 | ATGGACTACAAAGACGACGATGATAAGGCTAGCTCCTCCGACTCTCCGCGTAACCTG | Gene |
| | GAGGTTACCAACGCAACTCCGAACTCTCTGACTATTTCTTGGGACAATTCTAACTATG | sequence |
| | CTTCGTATTACCGTATCACCTACGGCGAAACCGGTGGTAACTCCCCGAGCCAGGAAC | encoding |
| | TCACTGTTCCGGGAAGTACTTATAATGCGACCATCAGCGGTCTGAAACCGGGCCAGG | aEpCAM-Fn3- |
| | ATTATATCATTACCGTGTACGCTGTAACCTATCGTGACAATTATTCCTATTCAAATCTA | DHFR²-CVIA |
| | ATCAGCATCAATTATCGCTCCGAAATCGACAAACCGTCTCAGGGATCCGGAGGTTCC | |
| | GGCGGGGGCGGAAGCGGAGGTGGAGGCTCAGGGGGGGGAGGGTCGGGCGGTGG | |
| | AGGTTCGGGGGGAGGCGGGAGCGGTGGCGGTGGTTCAGGAGGAGGGGGTTCCGG | |
| | GGGTGGTGGATCGGGCGGTGAGCTCGGCGGCATCAGTCTGATTGCGGCGTTAGCG | |
| | GTAGATCGCGTTATCGGCATGGAAAACGCCATGCCGTGGAACCTGCCTGCCGATCTC | |
| | GCCTGGTTTAAACGCAACACCTTAAATAAACCCGTGATTATGGGCCGCCATACCTGG | |
| | GAATCAATCGGTCGTCCGTTGCCAGGACGCAAAAATATTATCCTCAGCAGTCAACCG | |
| | GGTACGGACGATCGCGTAACGTGGGTGAAGTCGGTGGATGAAGCCATCGCGGCGG | |
| | CTGGTGACGTACCAGAAATCATGGTGATTGGCGGCGGTCGCGTTTATGAACAGTTCT | |
| | TGCCAAAAGCGCAAAAACTGTATCTGACGCATATCGACGCAGAAGTGGAAGGCGAC | |
| | ACCCATTTCCCGGATTACGAGCCGGATGACTGGGAATCGGTATTCAGTGAATTCCAC | |
| | GATGCTGATGCGCAGAACTCTCACAGCTATAGCTTTGAGATTCTGGAGCGGCGGGG | |
| | CGGCATTAGCCTTATTGCCGCCTTAGCGGTTGATCGCGTGATCGGAATGGAGAACG | |
| | CAATGCCCTGGAATCTTCCGGCAGACCTTGCCTGGTTCAAACGCAACACTTTAAACAA | |
| | GCCTGTCATTATGGGCCGTCACACATGGGAGTCAATTGGTCGTCCCCTGCCTGGGCG | |
| | CAAAAATATCATCTTGTCCTCGCAGCCTGGGACAGATGATCGCGTTACATGGGTGAA | |
| | GTCCGTAGACGAAGCGATTGCCGCTGCCGGCGATGTGCCCGAGATTATGGTAATCG | |
| | GGGGAGGGCGTGTTTACGAACAATTTCTGCCCAAAGCTCAGAAATTATACCTGACGC | |
| | ACATCGACGCGGAGGTCGAAGGTGACACACACTTTCCAGATTATGAGCCTGATGATT | |
| | GGGAATCCGTTTTCTCAGAATTTCATGACGCGGATGCTCAAAACTCGCACTCGTACTC | |
| | TTTTGAAATTTTAGAGCGCCGTGGCGGTTCAGGTGGTGGCTCGGGAGGCCATCATCA | |
| | TCATCATCACGGCGGATCTGGAGGAAGTGGCGGTAAAAAGAAAAAGAAAAAGACCT | |
| | GTGTCATCGCCTAGTGA | |
| 23 | ATGGACATTGTTCTCTCCCAGTCTCCAGCAATCATGTCTGCATCTCCAGGGGAGAAG | Gene |
| | GTCACCATATCCTGCAGTGCCAGCTCAAGTGTAAGTTATATGTACTGGTACCAGCAG | sequence |
| | AAGCCAGGATCCTCCCCCAAACCCTGGATTTATCGCACATCCAACCTGGCTTCTGGAG | encoding |
| | TCCCTGCTCGCTTCAGTGGCAGTGGGTCTGGGACCTCTTACTCTCTCACAATCAGCAG | aCD133-scFv- |
| | CATGGAGGCTGAAGATGCTGCCACTTATTACTGCCAGCAGTATCATAGTTACCCACCC | DHFR² |
| | ACGTTCGGTGCTGGGACCAAGCTGGAGCTGAAATCCTCTGGTGGCGGTGGCTCGGG | |
| | CGGTGGTGGGGGTGGTTCCTCTAGATCTTCCCTCGAGGTGAAGCTGGTGGAGTCTG | |
| | GACCTGAGCTGAAGAAGCCTGGAGAGACAGTCAAGATCTCCTGCAAGGCTTCTGGT | |
| | TATACCTTCACAGACTATTCAATGCACTGGGTGAATCAGGCTCCAGGAAAGGGTTTA | |
| | AAGTGGATGGGCTGGATAAACACTGAGACTGGTGAGCCATCATATGCAGATGACTT | |
| | CAAGGGACGGTTTGCCTTCTCTTTGGAAACCTCTGCCAGCACTGCCTATTTGCAGATC | |
| | AACAACCTCAAAAATGAGGACACGGCTACATATTTCTGTGCTACCGATTACGGGGAC | |
| | TACTTTGACTACTGGGGCCAAGGCACCACTCTCACAGTCTCCTCAGAGCTCGGCGGC | |
| | AGCGGCGGCGGCAGCGGCGGCGGCAGCGGCGGCATGATCAGTCTGATTGCGGCGT | |
| | TAGCGGTAGATCGCGTTATCGGCATGGAAAACGCCATGCCGTGGAACCTGCCTGCC | |
| | GATCTCGCCTGGTTTAAACGCAACACCTTAAATAAACCCGTGATTATGGGCCGCCATA | |
| | CCTGGGAATCAATCGGTCGTCCGTTGCCAGGACGCAAAAATATTATCCTCAGCAGTC | |
| | AACCGGGTACGGACGATCGCGTAACGTGGGTGAAGTCGGTGGATGAAGCCATCGC | |
| | GGCGGCTGGTGACGTACCAGAAATCATGGTGATTGGCGGCGGTCGCGTTTATGAAC | |
| | AGTTCTTGCCAAAAGCGCAAAAACTGTATCTGACGCATATCGACGCAGAAGTGGAA | |
| | GGCGACACCCATTTCCCGGATTACGAGCCGGATGACTGGGAATCGGTATTCAGTGAA | |
| | TTCCACGATGCTGATGCGCAGAACTCTCACAGCTATAGCTTTGAGATTCTGGAGCGG | |
| | CGGGGCATGATCAGTCTGATTGCGGCGCTAGCGGTAGATCGCGTTATCGGCATGGA | |
| | AAACGCCATGCCGTGGAACCTGCCTGCCGATCTCGCCTGGTTTAAACGCAACACCTT | |
| | AAATAAACCCGTGATTATGGGGCGCCATACCTGGGAATCAATCGGTAGGCCTTTGCC | |
| | CGGCCGCAAAAATATTATCCTCAGCAGTCAACCCGGGACCGATGATCGGGTTACCTG | |
| | GGTTAAATCGGTCGACGAAGCCATCGCGGCGGCTGGTGACGTACCAGAAATCATGG | |
| | TGATTGGCGGCGGACGCGTTTATGAACAGTTCTTGCCAAAAGCGCAAAAACTGTATC | |
| | TGACGCATATCGATGCAGAAGTGGAAGGCGACACCCATTTTCCGGATTACGAGCCG | |
| | GATGACTGGGAATCGGTATTCAGCGAGTTTCATGACGCGGACGCTCAGAACTCGCAT | |
| | AGCTATAGCTTCGAAATCCTGGAACGTCGTGGCGGCAGCGGCGGCGGCAGCGGCG | |
| | GCGGCAGCGGCGGCGACTACAAAGACGATGACGACAAGTAGTGA | |
| 24 | /5DBCOTEG/TGTCATATTCCTGGATCC/3AlexF488N/ | DBCO-ssDNA- |
| | | AF488 |
| 25 | TGTCATATTCCTGGATCCTTT/3DBCON/ | DBCO- |
| | | CTRLssDNA |
| 26 | T*G*T*C*A*T*A*T*T*C*C*T*G*G*A*T*C*C*T*T*T*/3DBCON/ | DBCO- |
| | | KDssDNA |
| | | Note: |
| | | * represents |
| | | phosphorothioate |
| | | bonds |
| | | modifications |
| | | of the backbone. |

### Conjugates

Certain embodiments of the invention provide a conjugate as described herein. In certain embodiments, the conjugate comprises a fusion protein as described herein. For example, in certain embodiments, the fusion protein comprises an amino acid sequence having at least 80% (e.g., 81%, 82%, 83%, 84%, 85%, 86%, 87%, 88%, 89%, 90%, 91%, 92%, 93%, 94%, 95%, 96%, 97%, 98%, 99%, or 100%) sequence identity to any of SEQ ID NO:14, 15, 16, or 18.

“Conjugate” as used herein refer to a fusion protein linked to a moiety that is not part of the N terminal to C terminal amino acid sequence of the fusion protein, wherein the moiety includes but is not limited to a Linker A (e.g., a lipid (e.g., an isoprenoid such as geranyl, farnesyl, or geranylgeranyl, or linker A is absent), a Linker B and a therapeutic agent, and/or a detectable agent. In certain embodiments, the moiety is directly linked to the fusion protein. In certain embodiments, the moiety is indirectly linked to the fusion protein (e.g., a therapeutic agent is indirectly linked to the fusion protein via a lipid such as farnesyl). In certain embodiments the fusion protein does not include the prenyltransferase substrate domain.

In certain embodiment linker A is a lipid. In certain embodiments linker A is a divalent, branched or unbranched, saturated or unsaturated, hydrocarbon chain having from 2 to 30 carbon atoms.

In certain embodiments the lipid is a hydrocarbon moiety. In certain embodiments the lipid is a hydrocarbon moiety having a molecular weight of about 14 daltons to about 700 daltons. In certain embodiments the lipid is a hydrocarbon moiety having a molecular weight of about 14 daltons to about 500 daltons. In certain embodiments the lipid is a hydrocarbon moiety having a molecular weight of about 28 daltons to about 700 daltons. In certain embodiments the lipid is a hydrocarbon moiety having a molecular weight of about 28 daltons to about 500 daltons. In certain embodiments the lipid is a divalent, branched or unbranched, saturated or unsaturated, hydrocarbon chain having from 2 to 50 carbon atoms. In certain embodiments the lipid is a divalent, branched or unbranched, saturated or unsaturated, hydrocarbon chain having from 2 to 50 carbon atoms. In certain embodiments the lipid is a divalent, branched or unbranched, saturated or unsaturated, hydrocarbon chain having from 5 to 50 carbon atoms.

In certain embodiments, the conjugate comprises a lipid. For example, after contacting with a prenyltransferase in the presence of a lipid, the fusion protein described herein may become a lipidated fusion protein.

In certain embodiments, the lipid comprises an azido group. In certain embodiments, the lipid does not comprise an azido group.

In certain embodiments, the lipid conjugated to the fusion protein is a geranyl group or derivative thereof (e.g., azido-geranyl). In certain embodiments, the lipid conjugated to the fusion protein is a geranyl derivative having structure:

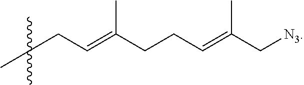 [SMILES: *C/C=C(\C)CC/C=C(\C)CN=[N+]=[N-]]

In certain embodiments, the lipid conjugated to the fusion protein is a farnesyl group or derivative thereof (e.g., azido-farnesyl). In certain embodiments, the lipid conjugated to the fusion protein is a farnesyl derivative having structure:

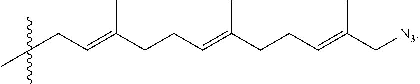 [SMILES: *C/C=C(\C)CC/C=C(\C)CC/C=C(\C)CN=[N+]=[N-]]

In certain embodiments, the lipid conjugated to the fusion protein is a geranylgeranyl group or derivative thereof (e.g., azido-geranylgeranyl). In certain embodiments, the lipid conjugated to the fusion protein is a geranylgeranyl derivative having structure:

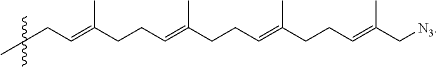 [SMILES: *C/C=C(\C)CC/C=C(\C)CC/C=C(\C)CC/C=C(\C)CN=[N+]=[N-]]

In certain embodiments, the lipid conjugated to the fusion protein is a geranyl derivative having structure:

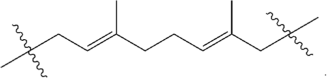 [SMILES: *C/C=C(\C)CC/C=C(\C)C*]

In certain embodiments, the lipid conjugated to the fusion protein is a farnesyl derivative having structure:

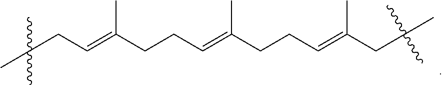 [SMILES: *C/C=C(\C)CC/C=C(\C)CC/C=C(\C)C*]

In certain embodiments, the lipid conjugated to the fusion protein is a geranylgeranyl derivative having structure:

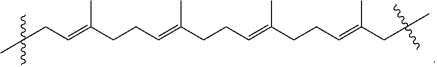 [SMILES: *C/C=C(\C)CC/C=C(\C)CC/C=C(\C)CC/C=C(\C)C*]

In certain embodiments the lipid is a geranyl group, a farnesyl group, a geranylgeranyl group, a palmitate group or a myristyl group.

In certain embodiments the lipid is the hydrocarbon (tail) portion of a geranyl group, a farnesyl group, a geranylgeranyl group, a palmitate group or a myristyl group.

In certain embodiments, linker A has a molecular weight of from about 20 daltons to about 20,000 daltons. In certain embodiments, linker A has a molecular weight of from about 20 daltons to about 10,000 daltons. In certain embodiments, linker A has a molecular weight of from about 20 daltons to about 5,000 daltons. In certain embodiments, linker A has a molecular weight of from about 20 daltons to about 3,000 daltons. In certain embodiments, linker A has a molecular weight of from about 20 daltons to about 2,000 daltons. In certain embodiments, linker A has a molecular weight of from about 20 daltons to about 1,000 daltons.

In certain embodiments, the linker A comprises a 5-6 membered heteroaryl (e.g., a triazolyl ring), or a multicyclic moiety (e.g., multicyclic heteroaryl or heterocyclyl) such as a multicyclic moiety comprising a triazolyl ring. In certain embodiments, the linker A comprises a divalent 10-20 membered multicyclic heteroaryl

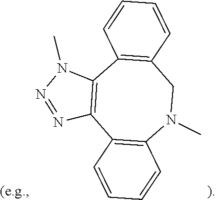 [SMILES: CN1Cc2ccccc2-c2c(nnn2C)-c2ccccc21]

In certain embodiments, linking could be achieved through DBCO functionalized molecule via a copper-free, strain-promoted alkyne/azide cycloaddition (SPAAC) involving the DBCO/azide groups.

In certain embodiments, linker A comprises a polyethylene glycol (PEG) segment with formula —(OCH₂CH₂)ₘ—, wherein m is an integer from 2 to 24 (e.g., m is 4).

In certain embodiments, linker A comprises enzymatically cleavable segment (e.g., a protease-sensitive segment, such as Cathepsin B sensitive segment).

In certain embodiments, linker A comprises a peptide segment. In certain embodiments, linker A comprises a peptide segment comprising 2, 3, 4, 5, or 6 amino acid residues. In certain embodiments, linker A comprises a peptide segment comprising Valine-Citrulline.

In certain embodiments, linker A has the structure:

—W—Z-T-Y—:
wherein W is selected from the group consisting of absent, a divalent 6-10 membered aryl or 5-20 membered heteroaryl (e.g., triazolyl or

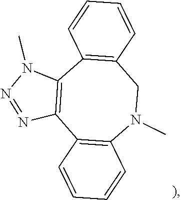 [SMILES: CN1Cc2ccccc2-c2c(nnn2C)-c2ccccc21]

—O—, —S—, —C(═O)—, —N(Rₐ)—, —C(═O)NH—, —C(═S)NH—, —C(═O)O—, —C(═O)S—, —NHSO₂—, —OC(═O)NH—, —NHC(═O)NH—, and —NHC(═S)NH—, wherein Rₐ is H or (C₁-C₆)alkyl;
Z is selected from the group consisting of absent, a peptide, or a divalent, branched or unbranched, saturated or unsaturated, hydrocarbon chain having from 2 to 30 carbon atoms, wherein one or more of the carbon atoms is optionally replaced by (—O—), (—S—), —N(R_{b})—, wherein R_{b} is H or (C₁-C₆)alkyl, wherein the hydrocarbon chain is optionally substituted on carbon with one or more substituents selected from the group consisting of halo, hydroxy, mercapto, oxo(═O), and thioxo(═S);
T is selected from the group consisting of absent, a peptide, or a divalent, branched or unbranched, saturated or unsaturated, hydrocarbon chain having from 2 to 30 carbon atoms, wherein one or more of the carbon atoms is optionally replaced by (—O—), (—S—), —N(R_{c})—, wherein R_{c} is H or (C₁-C₆)alkyl, wherein the hydrocarbon chain is optionally substituted on carbon with one or more substituents selected from the group consisting of halo, hydroxy, mercapto, oxo(═O), and thioxo(═S); and
Y is selected from the group consisting of absent, p-aminobenzyloxycarbonyl, —O—, —S—, —C(═O)—, —N(R_{d})—, —C(═O)NH—, —C(═S)NH—, —C(═O)O—, —C(═O)S—, —NHSO₂—, —OC(═O)NH—, —NHC(═O)NH—, and —NHC(═S)NH—, wherein R_{d} is H or (C₁-C₆)alkyl;
wherein W, Z, T, and Y are not simultaneously absent.

In certain embodiments, W is a divalent 10-20 membered multicyclic heteroaryl heterocyclyl.

In certain embodiments, Z is a polyethylene glycol (PEG) segment with formula —(OCH₂CH₂)ₘ—, wherein m is an integer from 2 to 6. In certain embodiments, m is 4.

In certain embodiments, Z is:

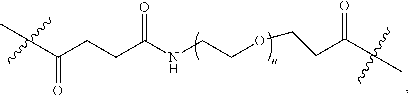

wherein n is an integer from 2 to 6. In certain embodiments, n is 4.

In certain embodiments, T is a peptide segment comprising 2, 3, 4, 5, or 6 amino acid residues. In certain embodiments, T is a peptide segment comprising 2 amino acid residues.

In certain embodiments, Y is p-aminobenzyloxycarbonyl.

In certain embodiments, linker A is

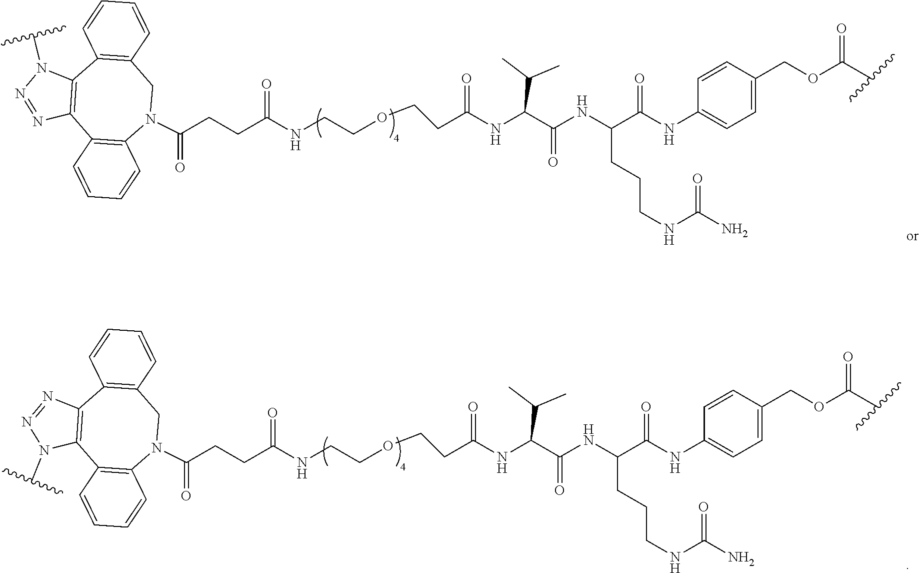

In certain embodiments, the linker A is a lipid or a moiety of structure —W—Z-T-Y—.

In certain embodiments, the linker B is moiety of structure —W—Z-T-Y—.

The term “therapeutic agent” includes agents that are useful for the treatment of a disease or a physiological condition in an animal (e.g., a mammal such as a human) and thus includes known drugs. Thus, the term “therapeutic agent” includes but is not limited to known drugs and/or drugs that have been approved for sale in the United States. For example, therapeutic agents include but are not limited to chemotherapeutic (anticancer) agents, antibiotic agents, antifungal agents, antiparasitic agents and antiviral agents.

The therapeutic agent may have activity when it is linked to the remainder of the conjugate or may become active when the linking group is hydrolyzed, and the therapeutic agent is released from the remainder of the conjugate.

In certain embodiments, the therapeutic agent is a small molecule compound with a molecular weight no greater than 1000 g/mol. In certain embodiments, the therapeutic agent is a peptide.

In certain embodiments, the therapeutic agent is an anti-cancer agent. In certain embodiments, the therapeutic agent is a cytotoxic agent (e.g., an apoptosis inducing agent). In certain embodiments, the therapeutic agent is a chemotherapeutic agent. In certain embodiments, the therapeutic agent is a chemotherapeutic agent belonging to the class of chalicheamicins. In certain embodiments, the therapeutic agent is a chemotherapeutic agent belonging to the class of auristatins (including but not limited to monomethyl auristatin E [MMAE], monomethyl auristatin F [MMAF], etc.). In certain embodiments, the therapeutic agent is MMAE. In certain embodiments, the therapeutic agent is a chemotherapeutic agent belonging to the class of maytansinoids (including but not limited to emtansine, also called DM1).

In certain embodiments, the therapeutic agent is a nucleic acid (e.g., DNA or RNA). In certain embodiments, the therapeutic agent is double stranded or single stranded nucleic acid. In certain embodiments, the therapeutic agent is a single stranded nucleic acid (e.g., ssDNA or ssRNA). In certain embodiments, the therapeutic agent is an antisense oligonucleotide (ASO). In certain embodiments, the therapeutic agent is a siRNA.

Detectable agents include, but are not limited to, fluorescent groups, and chelating groups, which may be labeled with radionuclides. A fluorophore is a molecule that absorbs light (i.e., excites) at a characteristic wavelength and emits light (i.e. fluoresces and emits a signal) at a second lower-energy wavelength. The detectable agent may include, but is not limited to, one or more of the following fluorescent groups: fluorescein, tetrachlorofluorescein, hexachlorofluorescein, tetramethylrhodamine, rhodamine, cyanine-derivative dyes, Texas Red, Bodipy, and Alexa dyes. Additional examples of certain fluorophores are listed at researchservices.umn.edu/sites/researchservices.umn.edu/files/configuration-lsrfortessa-h0081.pdf, which is incorporated by reference herein.

In certain embodiments, the detectable agent is a fluorescent dye. In certain embodiments, the detectable agent is a radioactive agent (e.g., a compound comprising ¹⁸F). In certain embodiments, the detectable agent comprises a chelating group labeled with a radionuclide. In certain embodiments, the detectable agent could be measured using imaging techniques such as fluorescent imaging or PET imaging.

The therapeutic agent or detectable agent can be bonded to the remainder of the conjugate as described herein by the removal of an atom such as a hydrogen atom from the therapeutic agent or detectable agent (e.g., a residue of an agent). Removal of the atom (e.g., hydrogen) provides the open valency to be connected to the remainder of the conjugate. In one embodiment the therapeutic agent or detectable agent comprises one or more hydroxyl, thiol, or amine (primary or secondary) groups which can be bonded to the conjugate. In one embodiment the term W is the residue of a therapeutic agent or detectable agent and the corresponding group H—W is the corresponding therapeutic agent or detectable agent. Thus, when the residue of the therapeutic agent or detectable agent (W) is released from the conjugate, the agent (H—W) is provided. In a similar manner the group —Z—W^{a} is a residue of an agent and the corresponding group H—Z—W^{a} is the corresponding agent. In one embodiment the Z of —Z—W^{a} is an oxygen atom, sulfur atom, NH or NR (R can be any group such as alkyl). Thus, one embodiment provides agents comprising one or more hydroxyl (—OH), thiol (—SH) or amine (e.g., primary (—NH₂) or secondary (—NH—, —NH(C₁-C₆)alkyl) groups which groups can be connected to remainder of the conjugate as described herein. In one embodiment the residue of the agent is derivable from an agent that comprises one or more groups selected from hydroxyl (OH), thiol (SH), primary amine (NH₂) and secondary amine (NH). In one embodiment the residue of the agent is derivable from an agent that comprises one or more hydroxyl (OH). In one embodiment the residue of the agent is derived from an agent that comprises one or more groups selected from hydroxyl (OH), thiol (SH), primary amine (NH₂) and secondary amine (NH). In one embodiment the residue of the agent is derived from an agent that comprises one or more hydroxyl (OH).

In certain embodiments, the conjugate comprises 1) a linker A, and 2) a therapeutic agent or detectable agent that is linked to the linker A via a linker B.

In certain embodiments, the conjugate comprises 1) a lipid, and 2) a therapeutic agent or detectable agent that is linked to the lipid via a linker.

In certain embodiments plurality comprises or includes 2, 3, 4, 5, 6, 7, 8, 9, 10 or more.

In certain embodiments, linker B has a molecular weight of from about 20 daltons to about 20,000 daltons. In certain embodiments, linker B has a molecular weight of from about 20 daltons to about 10,000 daltons. In certain embodiments, linker B has a molecular weight of from about 20 daltons to about 5,000 daltons. In certain embodiments, linker B has a molecular weight of from about 20 daltons to about 3,000 daltons. In certain embodiments, linker B has a molecular weight of from about 20 daltons to about 2,000 daltons. In certain embodiments, linker B has a molecular weight of from about 20 daltons to about 1,000 daltons.

In certain embodiments, linker B comprises a 5-6 membered heteroaryl (e.g., a triazolyl ring), or a multicyclic moiety (e.g., multicyclic heteroaryl or heterocyclyl) such as a multicyclic moiety comprising a triazolyl ring. In certain embodiments, the linker B comprises a divalent 10-20 membered multicyclic heteroaryl

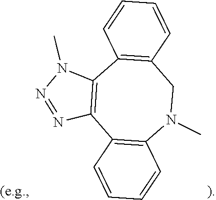 [SMILES: CN1Cc2ccccc2-c2c(nnn2C)-c2ccccc21]

In certain embodiments, linking could be achieved through DBCO functionalized molecule via a copper-free, strain-promoted alkyne/azide cycloaddition (SPAAC) involving the DBCO/azide groups.

In certain embodiments, linker B comprises a polyethylene glycol (PEG) segment with formula —(OCH₂CH₂)ₘ—, wherein m is an integer from 2 to 24 (e.g., m is 4).

In certain embodiments, Z is:

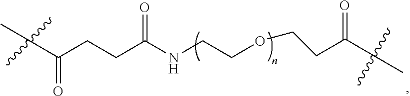

wherein n is an integer from 2 to 6. In certain embodiments, n is 4.

In certain embodiments, linker B comprises enzymatically cleavable segment (e.g., a protease-sensitive segment, such as Cathepsin B sensitive segment).

In certain embodiments, linker B comprises a peptide segment. In certain embodiments, the linker B comprises a peptide segment comprising 2, 3, 4, 5, or 6 amino acid residues. In certain embodiments, linker B comprises a peptide segment comprising Valine-Citrulline.

In certain embodiments, the linker B has the structure:

—W—Z-T-Y—
wherein: W is selected from the group consisting of absent, a divalent 6-10 membered aryl or 5-20 membered heteroaryl (e.g., triazolyl or

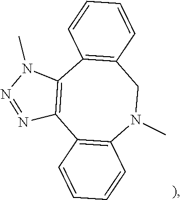 [SMILES: CN1Cc2ccccc2-c2c(nnn2C)-c2ccccc21]

—O—, —S—, —C(═O)—, —N(Rₐ)—, —C(═O)NH—, —C(═S)NH—, —C(═O)O—, —C(═O)S—, —NHSO₂—, —OC(═O)NH—, —NHC(═O)NH—, and —NHC(═S)NH—, wherein Rₐ is H or (C₁-C₆)alkyl;
Z is selected from the group consisting of absent, a peptide, or a divalent, branched or unbranched, saturated or unsaturated, hydrocarbon chain having from 2 to 30 carbon atoms, wherein one or more of the carbon atoms is optionally replaced by (—O—), (—S—), —N(R_{b})—, wherein R_{b} is H or (C₁-C₆)alkyl, wherein the hydrocarbon chain is optionally substituted on carbon with one or more substituents selected from the group consisting of halo, hydroxy, mercapto, oxo(═O), and thioxo(═S);
T is selected from the group consisting of absent, a peptide, or a divalent, branched or unbranched, saturated or unsaturated, hydrocarbon chain having from 2 to 30 carbon atoms, wherein one or more of the carbon atoms is optionally replaced by (—O—), (—S—), —N(R_{c})—, wherein R_{c} is H or (C₁-C₆)alkyl, wherein the hydrocarbon chain is optionally substituted on carbon with one or more substituents selected from the group consisting of halo, hydroxy, mercapto, oxo(═O), and thioxo(═S); and
Y is selected from the group consisting of absent, p-aminobenzyloxycarbonyl, —O—, —S—, —C(═O)—, —N(R_{d})—, —C(═O)NH—, —C(═S)NH—, —C(═O)O—, —C(═O)S—, —NHSO₂—, —OC(═O)NH—, —NHC(═O)NH—, and —NHC(═S)NH—, wherein R_{d} is H or (C₁-C₆)alkyl;
wherein W, Z, T, and Y are not simultaneously absent.

In certain embodiments, W is a divalent 10-20 membered multicyclic heteroaryl heterocyclyl.

In certain embodiments, Z is a polyethylene glycol (PEG) segment with formula —(OCH₂CH₂)ₘ—, wherein m is an integer from 2 to 6. In certain embodiments, m is 4.

In certain embodiments, T is a peptide segment comprising 2, 3, 4, 5, or 6 amino acid residues. In certain embodiments, T is a peptide segment comprising 2 amino acid residues.

In certain embodiments, Y is p-aminobenzyloxycarbonyl.

In certain embodiments, linker B is

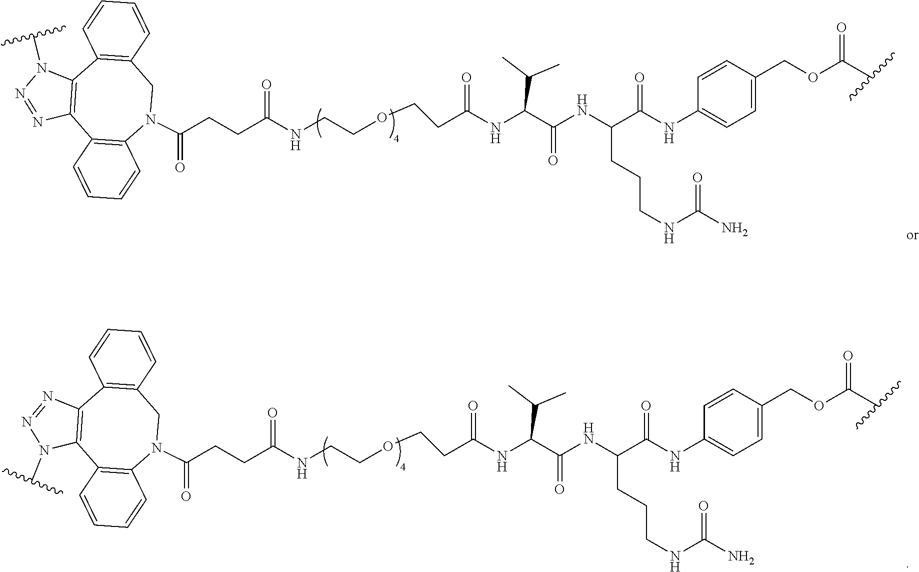

In certain embodiments, the conjugate (conjugate A1) comprises
A. a fusion protein comprising
   i. a targeting domain,
   ii. a first dihydrofolate reductase (DHFR) operably linked to a second DHFR, and
   iii. a prenyltransferase substrate domain;
B. a lipid operably linked to the prenyltransferase substrate domain of the fusion protein; and
C. a therapeutic agent or a detectable agent operably linked to the lipid.

In certain embodiments, the conjugate (conjugate A1) comprises
A. a fusion protein comprising
   i. a targeting domain,
   ii. a first dihydrofolate reductase (DHFR) operably linked to a second DHFR, and
   iii. a prenyltransferase substrate domain;
B. a linker A operably linked to the prenyltransferase substrate domain of the fusion protein; and
C. a therapeutic agent or a detectable agent operably linked to linker A with a linker B.

In certain embodiments, the conjugate (conjugate A1) comprises
A. a fusion protein comprising
   i. a targeting domain,
   ii. a first dihydrofolate reductase (DHFR) operably linked to a second DHFR, and
B. a therapeutic agent or a detectable agent operably linked to DHFR (e.g., the second DHFR) with a linker B.

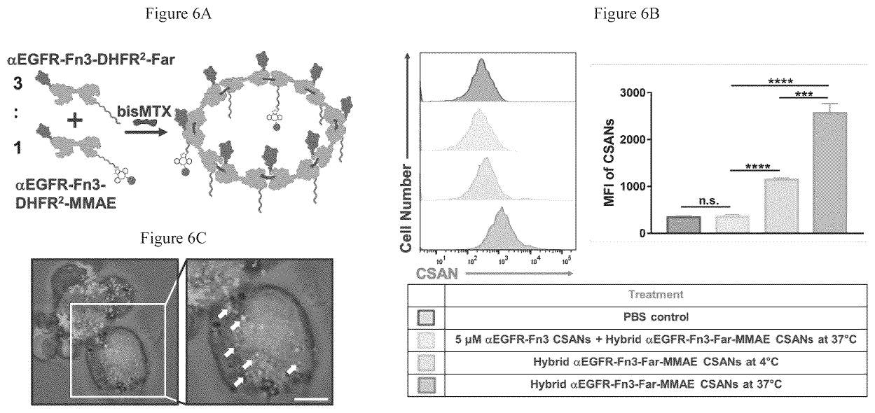

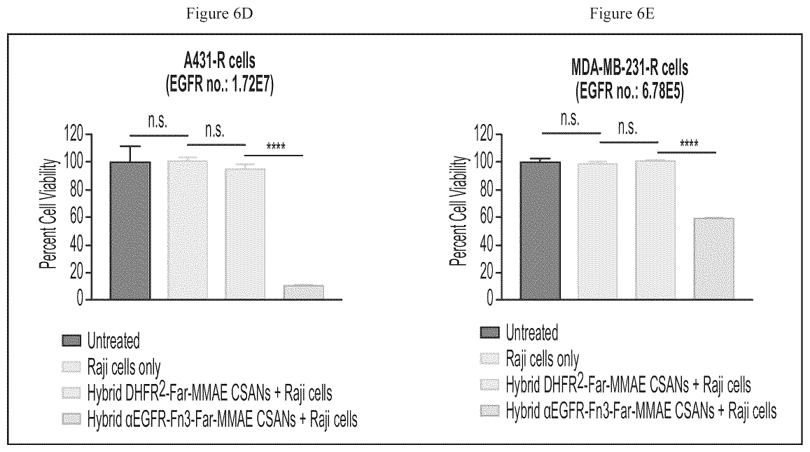

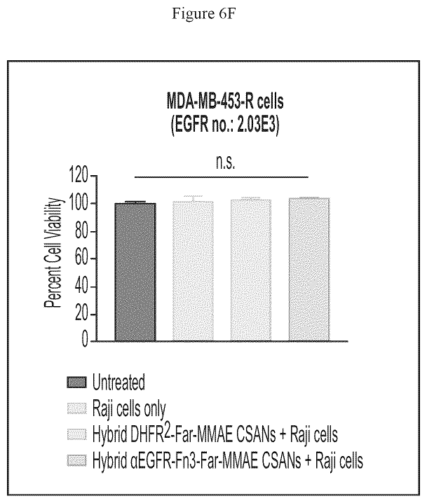

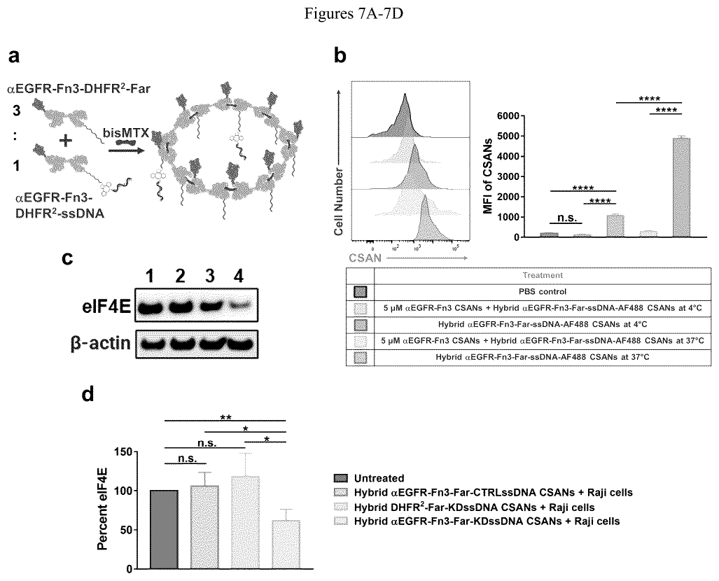

In certain embodiments, the conjugate (conjugate A1) comprises a fusion protein comprising an amino acid sequence having at least 80% sequence identity to SEQ ID NO:14, 15, or 16 (e.g., see αEGFR-Fn3-DHFR²-MMAE and FIG. 6a of Example 1; also see αEGFR-Fn3-DHFR²-ssDNA and FIG. 7a of Example 1).

In certain embodiments, the conjugate comprises a lipid such as geranyl, farnesyl, or geranylgeranyl and does not comprise a therapeutic agent or a detectable agent linked to the lipid.

In certain embodiments, the conjugate (conjugate A2) comprises
A. fusion protein comprising
   i. a targeting domain,
   ii. a first dihydrofolate reductase (DHFR) operably linked to a second DHFR, and
   iii. a prenyltransferase substrate domain; and
B. a lipid operably linked to the prenyltransferase substrate domain of the fusion protein.

In certain embodiments, the conjugate (conjugate A2) comprises
A. fusion protein comprising
   i. a targeting domain,
   ii. a first dihydrofolate reductase (DHFR) operably linked to a second DHFR, and
B. a lipid operably linked to DHFR (e.g., the second DHFR).

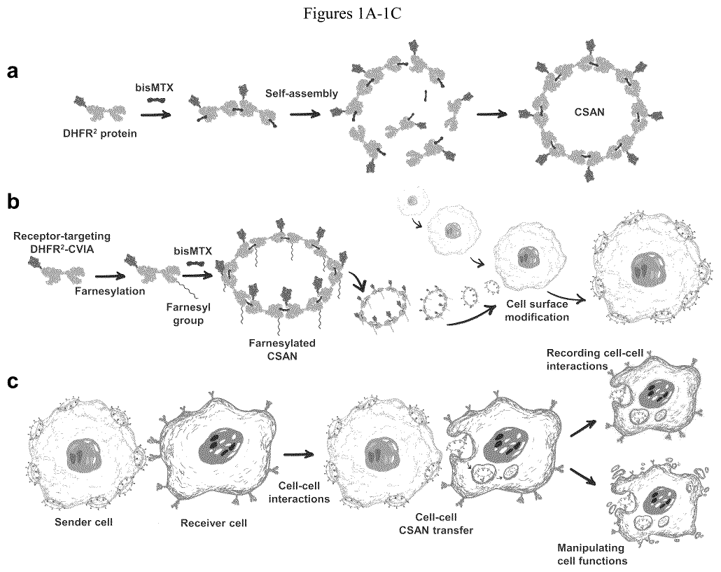

In certain embodiments, the conjugate (conjugate A2) comprises a fusion protein comprising an amino acid sequence having at least 80% sequence identity to SEQ ID NO:14, 15, or 16 (e.g., see FIG. 1b of Example 1; also see αEGFR-Fn3-DHFR²-Far and FIG. 6a or FIG. 7a of Example 1).

In certain embodiments, the conjugate does not comprise a targeting domain.

In certain embodiments, the conjugate (conjugate B1) comprises
A. fusion protein comprising
   i. a first dihydrofolate reductase (DHFR) operably linked to a second DHFR, and
   ii. a prenyltransferase substrate domain; and
B. a lipid operably linked to the prenyltransferase substrate domain of the fusion protein.

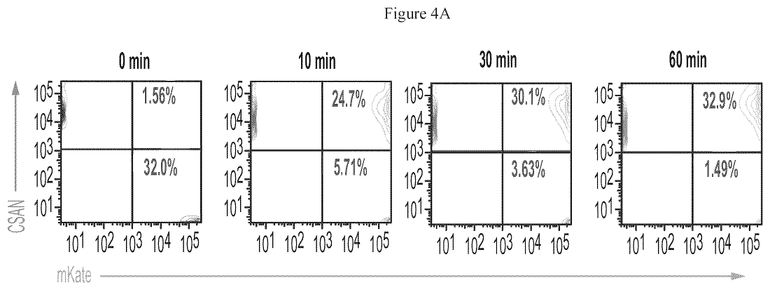

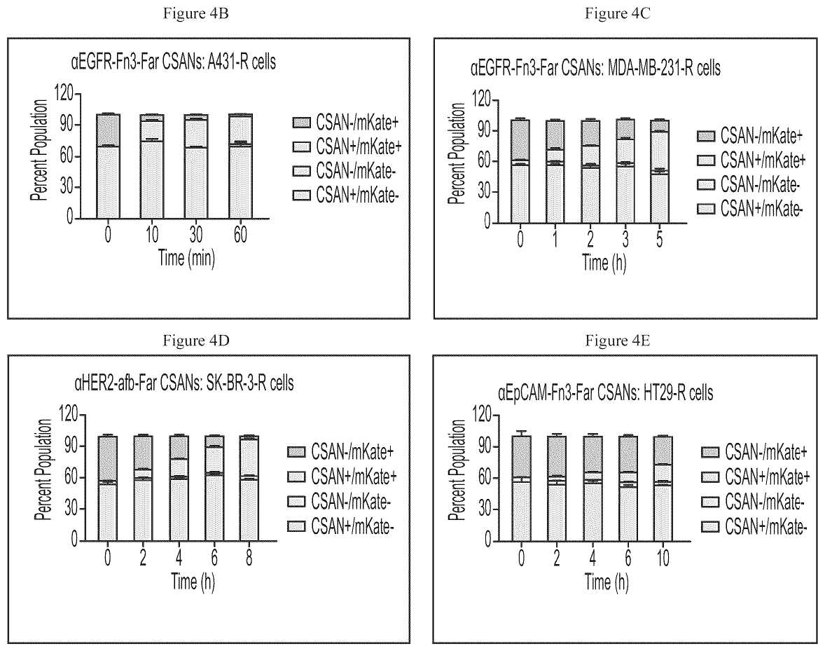

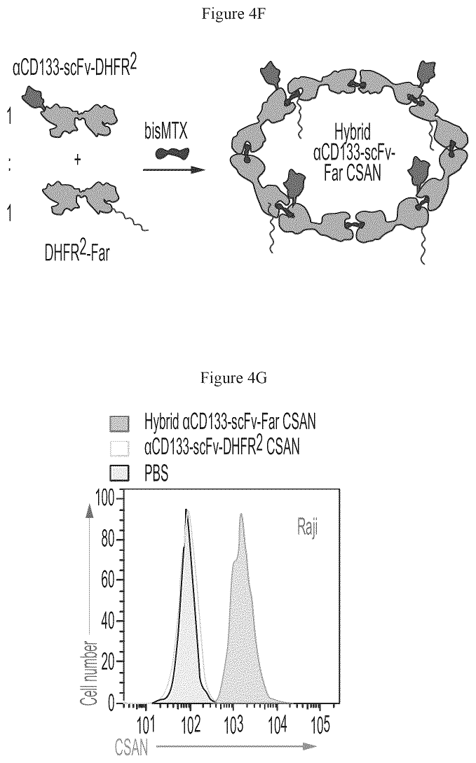

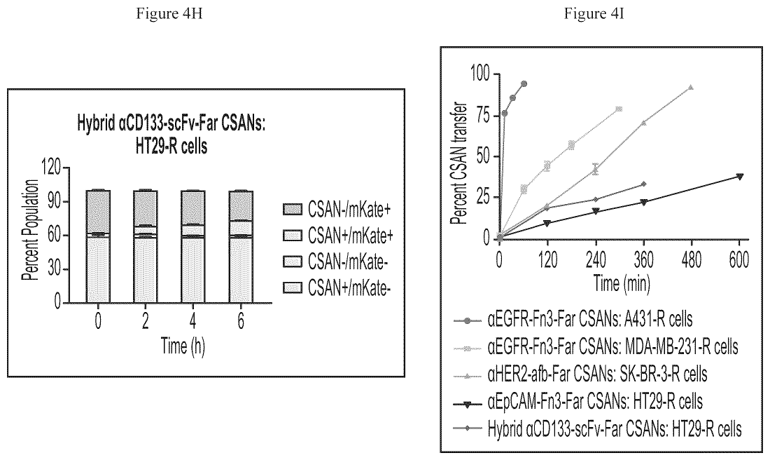

In certain embodiments, the conjugate (conjugate B1) comprises a fusion protein that does not comprise a targeting domain (e.g., see DHFR²-CVIA or SEQ ID NO:18 and FIG. 4f of Example 1).

In certain embodiments, conjugate B1 further comprises a therapeutic agent or a detectable agent operably linked to the lipid.

### Chemically Self-Assembled Nanoring (CSAN)

Certain embodiments of the invention provide a chemically self-assembled nanoring (CSAN) as described herein. In certain embodiments, the CSAN comprises a plurality of conjugates as described herein and a plurality of bisMTX compound.

As described herein, a conjugate of the invention may be incorporated into a chemically self-assembled nanoring (CSAN) and used for diagnostic or therapeutic purposes. A CSAN may be formed when conjugates of the invention are contacted with a chemical dimerizer (e.g., bis-methotrexate). As described herein, the nanoring is comprised of multiple fusion proteins, each comprising two subunits of dihydrofolate reductase (DHFR) joined by a peptide linker of variable length (e.g., 1-13 amino acids) and further fused to other domains (e.g., to a polypeptide described herein) and peptides (see, e.g., Carlson, J. C. T., et al. J. Am. Chem. Soc. 2006, 128, 7630-7638; Fegan, A., et al. Mol. Pharmaceutics. 2012, 9, 3218-3227; Li, Q., et al., J. Am. Chem. Soc. 2010, 132, 17247-17257; Shah, R, et al., Mol. Pharmaceutics. 2016, 13 (7), 2193-2203; Gangar, A., et al., J. Am. Chem. Soc. 2012, 134, 2895-2897; Shen, J., et al., J. Am. Chem. Soc. 2015, 137, 10108-10111; Qing, L., et al., Angew. Chem. Int. Ed. 2008, 47, 10179-10182; Gangar, A., et al., Mol. Pharmaceutics. 2013, 10, 3514-3518; Gabrielse, K., et al., Angew. Chem. Int. Ed. 2014, 53, 5112-5116. These documents are incorporated by reference in their entirety for all purposes).

Certain bisMTX compounds are known in the art. In certain embodiments, the bixMTX compound is a bixMTX compound described in, e.g., Carlson, J. C. T., et al. J. Am. Chem. Soc. 2006, 128, 7630-7638; Fegan, A., et al. Mol. Pharmaceutics. 2012, 9, 3218-3227; Li, Q., et al., J. Am. Chem. Soc. 2010, 132, 17247-17257; Shah, R, et al., Mol. Pharmaceutics. 2016, 13 (7), 2193-2203; Gangar, A., et al., J. Am. Chem. Soc. 2012, 134, 2895-2897; Shen, J., et al., J. Am. Chem. Soc. 2015, 137, 10108-10111; Qing, L., et al., Angew. Chem. Int. Ed. 2008, 47, 10179-10182; Gangar, A., et al., Mol. Pharmaceutics. 2013, 10, 3514-3518; Gabrielse, K., et al., Angew. Chem. Int. Ed. 2014, 53, 5112-5116; US Patent publication US2015-0343082, US Patent publication US2015-0017189, U.S. Pat. No. 8,236,925 or U.S. Pat. No. 8,580,921 (these documents are incorporated by reference herein for all purposes).

The plurality of bisMTX compounds may consist of a single type of bisMTX or may be a mixture of different types of compounds (e.g., 2, 3, 4, 5 or more types of compounds).

In certain embodiments, the CSAN is an octameric nanoring consisting of one or more types of conjugates.

In certain embodiments, the CSAN may consist of a single type of conjugate or may be a mixture of different types of conjugates (e.g., 2, 3, 4, 5 or more types of conjugates).

In certain embodiments, the CSAN comprises one or more of conjugate A1 as described herein. In certain embodiments, the CSAN comprises eight conjugate A1 as described herein.

In certain embodiments, the CSAN comprises one or more of conjugate A2 as described herein. In certain embodiments, the CSAN comprises eight conjugate A2 as described herein.

In certain embodiments, conjugate A2 lacks the same therapeutic agent or detectable agent comprised within conjugate A1. In certain embodiments, conjugate A2 does not comprise a therapeutic agent or detectable agent linked to the lipid.

In certain embodiments, the CSAN is a hybrid CSAN as described herein. For example, in certain embodiments, the CSAN comprises one or more of conjugate A1 as described herein and further comprises one or more of conjugate A2 as described herein. In certain embodiments, the hybrid CSAN has molar ratio of A1/A2 that is 1:7, 1:3, 3:5, or 1:1. In certain embodiments, the hybrid CSAN has molar ratio of A1/A2 that is 5:3, 3:1, or 7:1. In certain embodiments, the hybrid CSAN has molar ratio of A1/A2 that is 1:3. In certain embodiments the CSAN further comprises a plurality of bisMTX compounds,

Certain embodiments of the invention provide a chemically self-assembled nanoring (CSAN) comprising a plurality of conjugate B1 as described herein, a plurality of fusion protein B2, and a plurality of bisMTX compounds,
wherein conjugate B1 comprises:
   A. fusion protein comprising
      i. a first dihydrofolate reductase (DHFR) operably linked to a second DHFR, and
      ii. a prenyltransferase substrate domain; and
   B. a lipid operably linked to the prenyltransferase substrate domain of the fusion protein; wherein conjugate B1 does not comprise a targeting domain;
   and
wherein fusion protein B2 comprises:
   i. a targeting domain, and
   ii. a first dihydrofolate reductase (DHFR) operably linked to a second DHFR.

In certain embodiments, conjugate B1 further comprises a therapeutic agent or a detectable agent linked to the lipid.

In certain embodiments, the fusion protein B2 is a fusion protein that is not prenylated. In certain embodiments, the fusion protein B2 is a fusion protein as described herein that comprises a targeting domain and DHFR² but does not comprise a prenyltransferase substrate domain (e.g., does not comprise CVIA sequence).

In certain embodiments, the fusion protein B2 comprises a targeting domain comprising disulfide bond(s).

In certain embodiments, the fusion protein B2 comprises a targeting domain lacking disulfide bond(s).

In certain embodiments, the fusion protein B2 comprises an amino acid sequence having at least 80% sequence identity to SEQ ID NO:17 (e.g., see αCD133-scFv-DHFR2 and FIG. 4f).

In certain embodiments, the CSAN comprises one or more of conjugate B1 as described herein and further comprises one or more of fusion protein B2 as described herein. In certain embodiments, the hybrid CSAN has molar ratio of B1/B2 that is 1:7, 1:3, 3:5, or 1:1. In certain embodiments, the hybrid CSAN has molar ratio of B1/B2 that is 5:3, 3:1, or 7:1. In certain embodiments, the hybrid CSAN has molar ratio of B1/B2 that is 1:1.

### CSAN Modified Cells

Certain embodiments of the invention provide a cell modified with a CSAN described herein, wherein the cell membrane comprises a CSAN as described herein. In certain embodiments, the modified cell membrane has lipid rafts that are associated with the CSAN.

In certain embodiments, the CSAN comprises a fusion protein as described herein that is non-specifically labeled with a fluorescent dye.

In certain embodiments, the CSAN comprises a conjugate described herein comprising a therapeutic agent or a detectable agent that is linked to the lipid.

In certain embodiments, the cell is a mammalian cell (e.g., human cell).

In certain embodiments, the cell is an immune cell. For example, in certain embodiments, the cell is a T cell (e.g., CD8⁺ cytotoxic T cell) or a NK cell.

In certain embodiments, the cell is a mesenchymal stem cell. In certain embodiments, the cell is an endothelial cell.

In certain embodiments, the cell is contacted with a CSAN in vitro to form CSAN modified cell. In certain embodiments, CSAN modified cell could be administered to a subject (e.g., a mammal).

In certain embodiments, the cell is isolated from a subject (e.g., a mammal) and contacted with a CSAN ex vivo to form CSAN modified cell, which could be administered to the subject.

In certain embodiments, a cell is modified with a CSAN as described herein via incubating the cell with the CSAN in a buffer (e.g., PBS or saline) or cell culture medium. In certain embodiments, the incubation is conducted in a temperature of about 4 to 37° C., 10 to 35° C., or 15 to 30° C. (e.g., in room temperature or in about 20-25° C.). In certain embodiments, the incubation is conducted in a cell culture incubator at about 37° C., or on ice or in a refrigerator at about 4° C.

In certain embodiments, the incubation is conducted for about 1 minutes to 3 hours, 2 minutes to 2 hours, 5 to 90 minutes, 10 to 75 minutes, or 20 to 60 minutes. In certain embodiments, the incubation is conducted with rotation. In certain embodiments, the CSAN modified cell is washed or separated from unbound CSAN after the incubation (e.g., washing with fresh buffer or cell culture medium).

In certain embodiments, the cell is contacted with more than one type of CSAN. For example, in certain embodiments, the cell is contacted with two or more types of CSANs having affinity for different targets to form a dual-targeted or multi-targeted CSANs modified cell. For example, in certain embodiments, the cell is contacted with a first CSAN having a first targeting domain and a second CSAN having a second targeting domain to form a dual-targeted CSANs modified cell.

### Methods

Certain embodiments of the invention provide a method for treating cancer in an animal in need of, comprising administering a therapeutically effective amount of a CSAN modified sender cells as described herein to the animal (e.g., a mammal such as human).

Certain embodiments of the invention provide a method of intercellular delivery comprising
a) contacting a sender cell with a CSAN as described herein, and b) contacting the CSAN modified sender cell with a receiver cell, wherein a therapeutic agent or a detectable agent linked to a lipid comprised within the CSAN is delivered from the sender cell to the receiver cell.

In certain embodiments, the sender cell is a mammalian cell (e.g., human cell).

In certain embodiments, the sender cell is an immune cell. For example, in certain embodiments, the sender cell is a T cell (e.g., CD8⁺ cytotoxic T cell) or a NK cell.

In certain embodiments, the sender cell is a mesenchymal stem cell. In certain embodiments, the sender cell is an endothelial cell.

In certain embodiments, the sender cell is contacted with a CSAN in vitro to form CSAN modified sender cell. In certain embodiments, the sender cell is isolated from a subject (e.g., a mammal) and contacted with a CSAN ex vivo to form CSAN modified sender cell, which could be administered to the subject.

In certain embodiments, contacting a sender cell with a CSAN as described herein comprises incubating the sender cell with the CSAN in a buffer (e.g., PBS or saline) or cell culture medium. In certain embodiments, the incubation is conducted in a temperature of about 4 to 37° C., 10 to 35° C., or 15 to 30° C. (e.g., in room temperature or in about 20-25° C.). In certain embodiments, the incubation is conducted in a cell culture incubator at about 37° C., or on ice or in a refrigerator at about 4° C.

In certain embodiments, the incubation is conducted for about 1 minutes to 3 hours, 2 minutes to 2 hours, 5 to 90 minutes, 10 to 75 minutes, or 20 to 60 minutes. In certain embodiments, the incubation is conducted with rotation. In certain embodiments, the method further comprises washing off or separating unbound CSAN from the sender cell after the incubation (e.g., washing with fresh buffer or cell culture medium).

In certain embodiments, the receiver cell expresses a cell surface protein that the targeting domain of the fusion protein has affinity for. In certain embodiments, the receiver cell expresses HER2, EGFR, EpCAM, or CD133. In certain embodiments, the receiver cell expresses integrin αvβ3.

In certain embodiments, the receiver cell is a cancer cell (e.g., a malignant cell, or a CSC). In certain embodiments, the receiver cell is a carcinoma cell.

In certain embodiments, the receiver cell is an immune cell. For example, in certain embodiments, the receiver cell is a phagocytic cell or professional antigen presenting cell (e.g., macrophage).

In certain embodiments, the CSAN modified sender cell is contacted with the receiver cell in vitro.

In certain embodiments, the CSAN modified sender cell is contacted with the receiver cell in vivo. For example, in certain embodiments, contacting the CSAN modified sender cell with a receiver cell comprises administering the CSAN modified sender cell to a subject (e.g., a mammal). In certain embodiments, the subject has tumor. In certain embodiments, the CSAN modified sender cell is administered to the subject systemically or locally. In certain embodiments, the CSAN modified sender cell is administered to the subject intravenously, intramuscularly, subcutaneously, or intratumorally. In certain embodiments, the CSAN modified sender cell is administered near a lymph node of a subject and/or is administered intranodally.

In certain embodiments, a therapeutic agent linked to a lipid comprised within the CSAN is delivered from the sender cell to the receiver cell. In certain embodiments, the therapeutic agent is an agent described herein. For example, in certain embodiments, the therapeutic agent is an anti-cancer agent. In certain embodiments, the therapeutic agent is a cytotoxic agent (e.g., an apoptosis inducing drug such as monomethyl auristatin E (MMAE)). In certain embodiments, the therapeutic agent is nucleic acid sequence (e.g., DNA or RNA). In certain embodiments, the therapeutic agent is an antisense oligonucleotide or siRNA.

In certain embodiments, the viability of the receiver cell is reduced after contacted with CSAN modified sender cell.

In certain embodiments, the sender cell is not affected by the therapeutic agent or detectable agent linked to a lipid comprised within the CSAN. For example, in certain embodiments, the viability of the CSAN modified sender cell is not affected by the CSAN modification and the therapeutic agent or detectable agent linked to a lipid comprised within the CSAN (e.g., no difference in cell viability of the CSAN modified sender cell as compared to a control such as cell modified with CSAN lacking a therapeutic agent).

In certain embodiments, a CSAN modified sender cell delivers the therapeutic agent or detectable agent to two or more receiver cells. For example, a CSAN modified sender cell may have cell-cell interaction with more than one receiver cell and may deliver therapeutic agent or detectable agent to 1, 2, 3, 4, 5, 6, 7, 8, 9, 10, 20, 30, 40, 50, 60, 70, 80, 90, 100 or more receiver cells.

In certain embodiments, the therapeutic agent or the detectable agent linked to a lipid comprised within the CSAN is delivered to the receiver cell within about 60 minutes, 45 minutes, 30 minutes, or 10 minutes after the receiver cell is contacted with the CSAN modified sender cell.

In certain embodiments, the sender cell is contacted with two or more types of CSANs having affinity for different targets to form a dual-targeted or multi-targeted CSANs modified sender cell. For example, in certain embodiments, the sender cell is contacted with a first CSAN having a first targeting domain and a second CSAN having a second targeting domain to form a dual-targeted CSANs modified sender cell.

Certain embodiments of the invention provide a method for monitoring cell-cell interaction and/or intercellular cargo transfer as described herein, comprising a) contacting a sender cell with a CSAN as described herein, b) contacting the CSAN modified sender cell with a receiver cell, and 3) detecting a signal of a detectable agent comprised within the CSAN.

In certain embodiments, the detectable agent is directly linked to amino acid residue(s) of a fusion protein comprised within the CSAN as described herein. For example, in certain embodiments, CSAN is non-specifically labeled with a detectable agent (e.g., fluorescent dye).

In certain embodiments, the detectable agent is linked to a lipid comprised within the CSAN as described herein. In certain embodiments, the detectable agent linked to the lipid comprised within the CSAN is delivered from the sender cell to the receiver cell.

In certain embodiments, the method further comprises detecting one or more signal (e.g., fluorescent signal(s)) from the sender cell and/or receiver cell. For example, the sender cell, receiver cell may express fluorescent protein(s) or may be stained with specific cellular or organelle markers (e.g., nucleus, endosome and/or lysosome) for dual-color or multi-color imaging.

In certain embodiments, the detecting comprises detection using flow cytometry or fluorescent microscopy (e.g., confocal microscopy).

In certain embodiments, the detection is conducted about 60 minutes, 45 minutes, 30 minutes, or 10 minutes after the receiver cell is contacted with the CSAN modified sender cell.

### Administration

Certain embodiments of the invention provide a pharmaceutical composition comprising a CSAN modified cell described herein and a pharmaceutically acceptable excipient.

The CSAN modified cells described herein can be formulated as pharmaceutical compositions of cellular therapy and administered to a mammalian host, such as a human patient in a variety of forms adapted to the chosen route of administration, e.g., by intravenous, intramuscular, or intratumoral routes.

The CSAN modified cells may be administered by infusion or injection. Formulations of the CSAN modified cells can be prepared in solutions, such as isotonic saline, that are suitable for maintaining cell viability.

The amount of the CSAN modified cells, required for use in treatment will vary with the route of administration, the nature of the condition being treated and the age and condition of the patient and will be ultimately at the discretion of the attendant physician, clinician, and/or pharmacist.

The desired dose may conveniently be presented in a single dose or as divided doses administered at appropriate intervals, for example, as two, three, four or more sub-doses per day.

The sub-dose itself may be further divided, e.g., into a number of discrete loosely spaced administrations.

The CSAN modified cells of the invention can also be administered in combination with other therapeutic agents, for example, other agents that are useful for treating cancer. Examples of such agents include, but is not limited to, chemotherapeutic agents, small molecule targeted therapy drugs, immune checkpoint inhibitors, or other cell-based therapy.

The invention also provides a conjugate, a CSAN, or a CSAN modified cell as described herein for use in medical therapy.

The invention also provides a conjugate, a CSAN, or a CSAN modified cell as described herein for the prophylactic or therapeutic treatment of cancer.

The invention also provides the use of a conjugate, a CSAN, or a CSAN modified cell as described herein to prepare a medicament for treating cancer in an animal (e.g., a mammal such as a human) in need thereof. In certain embodiments, the cancer is a carcinoma.

### Kits

Certain embodiments of the invention provide a kit comprising:
1) a fusion protein, an enzyme (e.g., prenyltransferase), and/or a lipid, a therapeutic agent, and/or a detectable agent, as described herein; and
2) instructions for forming a conjugate described herein.

Certain embodiments of the invention provide a kit comprising:
1) a conjugate described herein;
2) instructions for assembling the conjugate to a CSAN; and
3) optionally instructions for contacting the CSAN with a cell.

Certain embodiments of the invention provide a kit comprising:
1) one or more CSAN described herein;
2) instructions for contacting the CSAN with a cell; and optionally instructions for administering the CSAN modified cell to an animal.

It is to be understood that one or more (e.g., 1, 2, 3, 4, 5 or more) of any of the embodiments provided herein may be combined together.

### Certain Definitions

The following definitions are used, unless otherwise described: halo or halogen is fluoro, chloro, bromo, or iodo. Alkyl and alkoxy, etc. denote both straight and branched groups but reference to an individual radical such as propyl embraces only the straight chain radical (a branched chain isomer such as isopropyl being specifically referred to).

As used herein, the term “(Cₐ-C_{b})alkyl” wherein a and b are integers refers to a straight or branched chain alkyl radical having from a to b carbon atoms. Thus when a is 1 and b is 6, for example, the term includes methyl, ethyl, n-propyl, isopropyl, n-butyl, isobutyl, sec-butyl, t-butyl, n-pentyl and n-hexyl.

The term “alkynyl” refers to an unsaturated alkyl radical having one or more triple bonds. Examples of such unsaturated alkyl groups ethynyl, 1- and 3-propynyl, 3-butynyl, and higher homologs and isomers.

The term “phenoxy” refers to a phenyl group attached to the remainder of the molecule via an oxygen atom (“oxy”).

The term “heteroaryloxy” refers to a heteroaryl group attached to the remainder of the molecule via an oxygen atom (“oxy”).

The term “aryl” as used herein refers to a single aromatic ring or a multiple condensed ring system wherein the ring atoms are carbon. For example, an aryl group can have 6 to 10 carbon atoms, or 6 to 12 carbon atoms. Aryl includes a phenyl radical. Aryl also includes multiple condensed ring systems (e.g., ring systems comprising 2 rings) having about 9 to 12 carbon atoms or 9 to 10 carbon atoms in which at least one ring is aromatic. Such multiple condensed ring systems may be optionally substituted with one or more (e.g., 1 or 2) oxo groups on any cycloalkyl portion of the multiple condensed ring system. It is to be understood that the point of attachment of a multiple condensed ring system, as defined above, can be at any position of the ring system including an aryl or a cycloalkyl portion of the ring. Typical aryl groups include, but are not limited to, phenyl, indenyl, naphthyl, 1, 2, 3, 4-tetrahydronaphthyl, anthracenyl, and the like.

The term “heteroaryl” as used herein refers to a single aromatic ring or a multiple condensed ring system. The term includes single aromatic rings of from about 1 to 6 carbon atoms and about 1-4 heteroatoms selected from the group consisting of oxygen, nitrogen and sulfur in the rings. The sulfur and nitrogen atoms may also be present in an oxidized form provided the ring is aromatic. Such rings include but are not limited to pyridyl, pyrimidinyl, oxazolyl or furyl. The term also includes multiple condensed ring systems (e.g. ring systems comprising 2 rings) wherein a heteroaryl group, as defined above, can be condensed with one or more heteroaryls (e.g., naphthyridinyl), heterocycles, (e.g., 1, 2, 3, 4-tetrahydronaphthyridinyl), cycloalkyls (e.g., 5,6,7,8-tetrahydroquinolyl) or aryls (e.g. indazolyl) to form a multiple condensed ring system. Such multiple condensed ring systems may be optionally substituted with one or more (e.g., 1 or 2) oxo groups on the cycloalkyl or heterocycle portions of the condensed ring. In one embodiment a monocyclic or bicyclic heteroaryl has 5 to 10 ring atoms comprising 1 to 9 carbon atoms and 1 to 4 heteroatoms. It is to be understood that the point of attachment of a multiple condensed ring system (as defined above for a heteroaryl) can be at any position of the multiple condensed ring system including a heteroaryl, heterocycle, aryl or cycloalkyl portion of the multiple condensed ring system and at any suitable atom of the multiple condensed ring system including a carbon atom and heteroatom (e.g., a nitrogen).

Exemplary heteroaryls include but are not limited to pyridyl, pyrrolyl, pyrazinyl, pyrimidinyl, pyridazinyl, pyrazolyl, thienyl, indolyl, imidazolyl, oxazolyl, thiazolyl, furyl, oxadiazolyl, thiadiazolyl, quinolyl, isoquinolyl, benzothiazolyl, benzoxazolyl, indazolyl, quinoxalyl, quinazolyl, 5,6,7,8-tetrahydroisoquinolinyl, benzofuranyl, benzimidazolyl and thianaphthenyl.

The term “heterocyclyl” or “heterocycle” as used herein refers to a single saturated or partially unsaturated ring or a multiple condensed ring system. The term includes single saturated or partially unsaturated rings (e.g., 3, 4, 5, 6 or 7-membered rings) from about 1 to 6 carbon atoms and from about 1 to 3 heteroatoms selected from the group consisting of oxygen, nitrogen and sulfur in the ring. The ring may be substituted with one or more (e.g., 1, 2 or 3) oxo groups and the sulfur and nitrogen atoms may also be present in their oxidized forms. Such rings include but are not limited to azetidinyl, tetrahydrofuranyl or piperidinyl. It is to be understood that the point of attachment for a heterocycle can be at any suitable atom of the heterocycle Exemplary heterocycles include, but are not limited to aziridinyl, azetidinyl, pyrrolidinyl, piperidinyl, homopiperidinyl, morpholinyl, thiomorpholinyl, piperazinyl, tetrahydrofuranyl, dihydrooxazolyl, tetrahydropyranyl and tetrahydrothiopyranyl.

The term cycloalkyl, carbocycle, or carbocyclyl includes saturated and partially unsaturated carbocyclic ring systems. In one embodiment the cycloalkyl is a monocyclic carbocyclic ring. Such cycloalkyls include “(C₃-C₇)carbocyclyl” and “(C₃-C₈)cycloalkyl”.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₁-C₆)alkoxy can be methoxy, ethoxy, propoxy, isopropoxy, butoxy, iso-butoxy, sec-butoxy, pentoxy, 3-pentoxy, or hexyloxy; (C₃-C₈)cycloalkyl can be cyclopropyl, cyclobutyl, cyclopentyl, or cyclohexyl; (C₁-C₆)haloalkyl can be iodomethyl, bromomethyl, chloromethyl, fluoromethyl, trifluoromethyl, 2-chloroethyl, 2-fluoroethyl, 2,2,2-trifluoroethyl, or pentafluoroethyl; aryl can be phenyl, indenyl, or naphthyl; and heteroaryl can be furyl, imidazolyl, triazolyl, triazinyl, oxazolyl, isoxazolyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, isoquinolyl (or its N-oxide) or quinolyl (or its N-oxide).

The term “nucleic acid” refers to deoxyribonucleotides or ribonucleotides and polymers thereof in either single- or double-stranded form, composed of monomers (nucleotides) containing a sugar, phosphate and a base which is either a purine or pyrimidine. Unless specifically limited, the term encompasses nucleic acids containing known analogs of natural nucleotides that have similar binding properties as the reference nucleic acid and are metabolized in a manner similar to naturally occurring nucleotides. Unless otherwise indicated, a particular nucleic acid sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxyinosine residues (Batzer et al. (1991) Nucl. Acids Res., 19:508; Ohtsuka et al. (1985) JBC, 260:2605; Rossolini et al. (1994) Mol. Cell. Probes, 8:91. A “nucleic acid fragment” is a fraction of a given nucleic acid molecule. Deoxyribonucleic acid (DNA) in the majority of organisms is the genetic material while ribonucleic acid (RNA) is involved in the transfer of information contained within DNA into proteins. The term “nucleotide sequence” refers to a polymer of DNA or RNA that can be single- or double-stranded, optionally containing synthetic, non-natural or altered nucleotide bases capable of incorporation into DNA or RNA polymers. The terms “nucleic acid,” “nucleic acid molecule,” “nucleic acid fragment,” “nucleic acid sequence or segment,” or “polynucleotide” may also be used interchangeably with gene, cDNA, DNA and RNA encoded by a gene.

By “portion” or “fragment,” as it relates to a nucleic acid molecule, sequence or segment of the invention, when it is linked to other sequences for expression, is meant a sequence having at least 80 nucleotides, more preferably at least 150 nucleotides, and still more preferably at least 400 nucleotides. If not employed for expressing, a “portion” or “fragment” means at least 9, preferably 12, more preferably 15, even more preferably at least 20, consecutive nucleotides, e.g., probes and primers (oligonucleotides), corresponding to the nucleotide sequence of the nucleic acid molecules of the invention.

The term “amino acid,” comprises the residues of the natural amino acids (e.g. Ala, Arg, Asn, Asp, Cys, Glu, Gln, Gly, His, Hyl, Hyp, Ile, Leu, Lys, Met, Phe, Pro, Ser, Thr, Trp, Tyr, and Val) in D or L form, as well as unnatural amino acids (e.g. phosphoserine, phosphothreonine, phosphotyrosine, hydroxyproline, gamma-carboxyglutamate; hippuric acid, octahydroindole-2-carboxylic acid, statine, 1,2,3,4,-tetrahydroisoquinoline-3-carboxylic acid, penicillamine, ornithine, citruline, α-methyl-alanine, para-benzoylphenylalanine, phenylglycine, propargylglycine, sarcosine, and tert-butylglycine). The term also comprises natural and unnatural amino acids bearing a conventional amino protecting group (e.g. acetyl or benzyloxycarbonyl), as well as natural and unnatural amino acids protected at the carboxy terminus (e.g. as a (C₁-C₆) alkyl, phenyl or benzyl ester or amide; or as an α-methylbenzyl amide). Other suitable amino and carboxy protecting groups are known to those skilled in the art (See for example, T. W. Greene, Protecting Groups In Organic Synthesis; Wiley: New York, 1981, and references cited therein).

The term “peptide” describes a sequence of 2 to 25 amino acids (e.g. as defined herein above) or peptidyl residues. The sequence may be linear or cyclic. For example, a cyclic peptide can be prepared or may result from the formation of amide bonds or disulfide bridges between two cysteine residues in a sequence. A peptide can be linked to the remainder of a fusion protein or conjugate through the carboxy terminus, the amino terminus, or through any other convenient point of attachment, such as via side chain of the amino acid, for example, through the sulfur of a cysteine. In certain embodiments, a peptide comprises 2 to 10, or 3 to 8 amino acids. In certain embodiments, a peptide comprises 5 to 13 amino acids, or 5 to 9 amino acids. Peptide derivatives can be prepared as disclosed in U.S. Pat. Nos. 4,612,302; 4,853,371; and 4,684,620.

The terms “protein,” and “polypeptide” are used interchangeably herein. Polypeptide sequences specifically recited herein are written with the amino terminus on the left and the carboxy terminus on the right.

The invention encompasses isolated or substantially purified nucleic acid or protein compositions. In the context of the present invention, an “isolated” or “purified” DNA molecule or an “isolated” or “purified” polypeptide is a DNA molecule or polypeptide that exists apart from its native environment and is therefore not a product of nature. An isolated DNA molecule or polypeptide may exist in a purified form or may exist in a non-native environment such as, for example, a transgenic host cell. For example, an “isolated” or “purified” nucleic acid molecule or protein, or biologically active portion thereof, is substantially free of other cellular material, or culture medium when produced by recombinant techniques, or substantially free of chemical precursors or other chemicals when chemically synthesized. In one embodiment, an “isolated” nucleic acid is free of sequences that naturally flank the nucleic acid (i.e., sequences located at the 5′ and 3′ ends of the nucleic acid) in the genomic DNA of the organism from which the nucleic acid is derived. For example, in various embodiments, the isolated nucleic acid molecule can contain less than about 5 kb, 4 kb, 3 kb, 2 kb, 1 kb, 0.5 kb, or 0.1 kb of nucleotide sequences that naturally flank the nucleic acid molecule in genomic DNA of the cell from which the nucleic acid is derived. A protein that is substantially free of cellular material includes preparations of protein or polypeptide having less than about 30%, 20%, 10%, 5%, (by dry weight) of contaminating protein. When the protein of the invention, or biologically active portion thereof, is recombinantly produced, preferably culture medium represents less than about 30%, 20%, 10%, or 5% (by dry weight) of chemical precursors or non-protein-of-interest chemicals. Fragments and variants of the disclosed nucleotide sequences and proteins or partial-length proteins encoded thereby are also encompassed by the present invention. By “fragment” or “portion” is meant a full length or less than full length of the nucleotide sequence encoding, or the amino acid sequence of, a polypeptide or protein.

“Naturally occurring” is used to describe an object that can be found in nature as distinct from being artificially produced. For example, a protein or nucleotide sequence present in an organism (including a virus), which can be isolated from a source in nature and which has not been intentionally modified by man in the laboratory, is naturally occurring.

“Wild-type” refers to the normal gene, or organism found in nature without any known mutation.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of well-known molecular biology techniques, as, for example, with polymerase chain reaction (PCR) and hybridization techniques. Variant nucleotide sequences also include synthetically derived nucleotide sequences, such as those generated, for example, by using site-directed mutagenesis that encode the native protein, as well as those that encode a polypeptide having amino acid substitutions. Generally, nucleotide sequence variants of the invention will have at least 40, 50, 60, to 70%, e.g., preferably 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence.

“Conservatively modified variations” of a particular nucleic acid sequence refers to those nucleic acid sequences that encode identical or essentially identical amino acid sequences, or where the nucleic acid sequence does not encode an amino acid sequence, to essentially identical sequences. Because of the degeneracy of the genetic code, a large number of functionally identical nucleic acids encode any given polypeptide. For instance the codons CGT, CGC, CGA, CGG, AGA, and AGG all encode the amino acid arginine. Thus, at every position where an arginine is specified by a codon, the codon can be altered to any of the corresponding codons described without altering the encoded protein. Such nucleic acid variations are “silent variations” which are one species of “conservatively modified variations.” Every nucleic acid sequence described herein which encodes a polypeptide also describes every possible silent variation, except where otherwise noted. One of skill will recognize that each codon in a nucleic acid (except ATG, which is ordinarily the only codon for methionine) can be modified to yield a functionally identical molecule by standard techniques. Accordingly, each “silent variation” of a nucleic acid which encodes a polypeptide is implicit in each described sequence.

“Recombinant DNA molecule” is a combination of DNA sequences that are joined together using recombinant DNA technology and procedures used to join together DNA sequences as described, for example, in Sambrook and Russell, Molecular Cloning: A Laboratory Manual, Cold Spring Harbor, NY: Cold Spring Harbor Laboratory Press (3^{rd} edition, 2001).

The terms “heterologous DNA sequence,” “exogenous DNA segment” or “heterologous nucleic acid,” each refer to a sequence that originates from a source foreign to the particular host cell or, if from the same source, is modified from its original form. Thus, a heterologous gene in a host cell includes a gene that is endogenous to the particular host cell but has been modified.

The terms also include non-naturally occurring multiple copies of a naturally occurring DNA sequence. Thus, the terms refer to a DNA segment that is foreign or heterologous to the cell, or homologous to the cell but in a position within the host cell nucleic acid in which the element is not ordinarily found. Exogenous DNA segments are expressed to yield exogenous polypeptides.

A “homologous” DNA sequence is a DNA sequence that is naturally associated with a host cell into which it is introduced.

A “vector” is defined to include, inter alia, any viral vector, plasmid, cosmid, phage or binary vector in double or single stranded linear or circular form which may or may not be self-transmissible or mobilizable, and which can transform prokaryotic or eukaryotic host either by integration into the cellular genome or exist extrachromosomally (e.g., autonomous replicating plasmid with an origin of replication).

“Cloning vectors” typically contain one or a small number of restriction endonuclease recognition sites at which foreign DNA sequences can be inserted in a determinable fashion without loss of essential biological function of the vector, as well as a marker gene that is suitable for use in the identification and selection of cells transformed with the cloning vector. Marker genes typically include genes that provide tetracycline resistance, hygromycin resistance or ampicillin resistance.

“Expression cassette” as used herein means a DNA sequence capable of directing expression of a particular nucleotide sequence in an appropriate host cell, comprising a promoter operably linked to the nucleotide sequence of interest which is operably linked to termination signals. It also typically comprises sequences required for proper translation of the nucleotide sequence. The coding region usually codes for a protein of interest but may also code for a functional RNA of interest, for example antisense RNA or a nontranslated RNA, in the sense or antisense direction. The expression cassette comprising the nucleotide sequence of interest may be chimeric, meaning that at least one of its components is heterologous with respect to at least one of its other components. The expression cassette may also be one that is naturally occurring but has been obtained in a recombinant form useful for heterologous expression. The expression of the nucleotide sequence in the expression cassette may be under the control of a constitutive promoter or of an inducible promoter that initiates transcription only when the host cell is exposed to some particular external stimulus. In the case of a multicellular organism, the promoter can also be specific to a particular tissue or organ or stage of development.

Such expression cassettes will comprise the transcriptional initiation region of the invention linked to a nucleotide sequence of interest. Such an expression cassette is provided with a plurality of restriction sites for insertion of the gene of interest to be under the transcriptional regulation of the regulatory regions. The expression cassette may additionally contain selectable marker genes.

The term “RNA transcript” refers to the product resulting from RNA polymerase catalyzed transcription of a DNA sequence. When the RNA transcript is a perfect complimentary copy of the DNA sequence, it is referred to as the primary transcript or it may be a RNA sequence derived from posttranscriptional processing of the primary transcript and is referred to as the mature RNA. “Messenger RNA” (mRNA) refers to the RNA that is without introns and that can be translated into protein by the cell. “cDNA” refers to a single- or a double-stranded DNA that is complementary to and derived from mRNA.

“Regulatory sequences” and “suitable regulatory sequences” each refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences include enhancers, promoters, translation leader sequences, introns, and polyadenylation signal sequences. They include natural and synthetic sequences as well as sequences that may be a combination of synthetic and natural sequences. As is noted above, the term “suitable regulatory sequences” is not limited to promoters. However, some suitable regulatory sequences useful in the present invention will include, but are not limited to constitutive promoters, tissue-specific promoters, development-specific promoters, inducible promoters and viral promoters.

“5′ non-coding sequence” refers to a nucleotide sequence located 5′ (upstream) to the coding sequence. It is present in the fully processed mRNA upstream of the initiation codon and may affect processing of the primary transcript to mRNA, mRNA stability or translation efficiency (Turner et al. (1995) Mol. Biotech. 3:225).

“3′ non-coding sequence” refers to nucleotide sequences located 3′ (downstream) to a coding sequence and include polyadenylation signal sequences and other sequences encoding regulatory signals capable of affecting mRNA processing or gene expression. The polyadenylation signal is usually characterized by affecting the addition of polyadenylic acid tracts to the 3′ end of the mRNA precursor.

The term “translation leader sequence” refers to that DNA sequence portion of a gene between the promoter and coding sequence that is transcribed into RNA and is present in the fully processed mRNA upstream (5′) of the translation start codon. The translation leader sequence may affect processing of the primary transcript to mRNA, mRNA stability or translation efficiency.

The term “mature” protein refers to a post-translationally processed polypeptide without its signal peptide. “Precursor” protein refers to the primary product of translation of an mRNA. “Signal peptide” refers to the amino terminal extension of a polypeptide, which is translated in conjunction with the polypeptide forming a precursor peptide and which is required for its entrance into the secretory pathway. The term “signal sequence” refers to a nucleotide sequence that encodes the signal peptide.

“Promoter” refers to a nucleotide sequence, usually upstream (5′) to its coding sequence, which controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements that is capable of controlling the expression of a coding sequence or functional RNA. This type of promoter sequence consists of proximal and more distal upstream elements, the latter elements often referred to as enhancers. Accordingly, an “enhancer” is a DNA sequence that can stimulate promoter activity and may be an innate element of the promoter or a heterologous element inserted to enhance the level or tissue specificity of a promoter. Promoters may be derived in their entirety from a native gene, or be composed of different elements derived from different promoters found in nature, or even be comprised of synthetic DNA segments. A promoter may also contain DNA sequences that are involved in the binding of protein factors that control the effectiveness of transcription initiation in response to physiological or developmental conditions.

The “initiation site” is the position surrounding the first nucleotide that is part of the transcribed sequence, which is also defined as position+1. With respect to this site all other sequences of the gene and its controlling regions are numbered. Downstream sequences (i.e. further protein encoding sequences in the 3′ direction) are denominated positive, while upstream sequences (mostly of the controlling regions in the 5′ direction) are denominated negative.

Promoter elements, particularly a TATA element, that are inactive or that have greatly reduced promoter activity in the absence of upstream activation are referred to as “minimal or core promoters.” In the presence of a suitable transcription factor, the minimal promoter functions to permit transcription. A “minimal or core promoter” thus consists only of all basal elements needed for transcription initiation, e.g., a TATA box and/or an initiator.

As used herein, the term “operably linked” refers to a linkage of two elements in a functional relationship. For example, “operably linked” may refer to a linkage of polynucleotide elements or polypeptide elements in a functional relationship. A nucleic acid is “operably linked” when it is placed into a functional relationship with another nucleic acid sequence. For example, a regulatory DNA sequence is said to be “operably linked to” or “associated with” a DNA sequence that codes for an RNA or a polypeptide if the two sequences are situated such that the regulatory DNA sequence affects expression of the coding DNA sequence (i.e., that the coding sequence or functional RNA is under the transcriptional control of the promoter).

Coding sequences can be operably-linked to regulatory sequences in sense or antisense orientation. “Operably-linked” also refers to the association of two chemical moieties so that the function of one is affected by the other, e.g., an arrangement of elements wherein the components so described are configured so as to perform their usual function.

“Expression” refers to the transcription and/or translation in a cell of an endogenous gene, transgene, as well as the transcription and stable accumulation of sense (mRNA) or functional RNA. In the case of antisense constructs, expression may refer to the transcription of the antisense DNA only. Expression may also refer to the production of protein.

“Transcription stop fragment” refers to nucleotide sequences that contain one or more regulatory signals, such as polyadenylation signal sequences, capable of terminating transcription. Examples of transcription stop fragments are known to the art.

“Translation stop fragment” refers to nucleotide sequences that contain one or more regulatory signals, such as one or more termination codons in all three frames, capable of terminating translation. Insertion of a translation stop fragment adjacent to or near the initiation codon at the 5′ end of the coding sequence will result in no translation or improper translation. Excision of the translation stop fragment by site-specific recombination will leave a site-specific sequence in the coding sequence that does not interfere with proper translation using the initiation codon.

The following terms are used to describe the sequence relationships between two or more sequences (e.g., nucleic acids, polynucleotides or polypeptides): (a) “reference sequence,” (b) “comparison window,” (c) “sequence identity,” (d) “percentage of sequence identity,” and (e) “substantial identity.”
(a) As used herein, “reference sequence” is a defined sequence used as a basis for sequence comparison. A reference sequence may be a subset or the entirety of a specified sequence; for example, as a segment of a full length cDNA, gene sequence or peptide sequence, or the complete cDNA, gene sequence or peptide sequence.
(b) As used herein, “comparison window” makes reference to a contiguous and specified segment of a sequence, wherein the sequence in the comparison window may comprise additions or deletions (i.e., gaps) compared to the reference sequence (which does not comprise additions or deletions) for optimal alignment of the two sequences. Generally, the comparison window is at least 20 contiguous nucleotides in length, and optionally can be 30, 40, 50, 100, or longer. Those of skill in the art understand that to avoid a high similarity to a reference sequence due to inclusion of gaps in the sequence a gap penalty is typically introduced and is subtracted from the number of matches.

Methods of alignment of sequences for comparison are well known in the art. Thus, the determination of percent identity between any two sequences can be accomplished using a mathematical algorithm. Non-limiting examples of such mathematical algorithms are the algorithm of Myers and Miller (1988) CABIOS, 4:11; the local homology algorithm of Smith et al. (1981) Adv. Appl. Math. 2:482; the homology alignment algorithm of Needleman and Wunsch, (1970) JMB, 48:443; the search-for-similarity-method of Pearson and Lipman, (1988) Proc. Natl. Acad. Sci. USA, 85:2444; the algorithm of Karlin and Altschul, (1990) Proc. Natl. Acad. Sci. USA, 87:2264, modified as in Karlin and Altschul, (1993) Proc. Natl. Acad. Sci. USA, 90:5873.

Computer implementations of these mathematical algorithms can be utilized for comparison of sequences to determine sequence identity. Such implementations include, but are not limited to: CLUSTAL in the PC/Gene program (available from Intelligenetics, Mountain View, California); the ALIGN program (Version 2.0) and GAP, BESTFIT, BLAST, FASTA, and TFASTA in the Wisconsin Genetics Software Package, Version 8 (available from Genetics Computer Group (GCG), 575 Science Drive, Madison, Wisconsin, USA). Alignments using these programs can be performed using the default parameters. The CLUSTAL program is well described by Higgins et al. (1988) Gene 73:237; Higgins et al. (1989) CABIOS 5:151; Corpet et al. (1988) Nucl. Acids Res. 16:10881; Huang et al. (1992) CABIOS 8:155; and Pearson et al. (1994) Meth. Mol. Biol. 24:307. The ALIGN program is based on the algorithm of Myers and Miller, supra. The BLAST programs of Altschul et al. (1990) JMB, 215:403; Nucl. Acids Res., 25:3389 (1990), are based on the algorithm of Karlin and Altschul supra.

Software for performing BLAST analyses is publicly available through the National Center for Biotechnology Information (available on the world wide web at ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold. These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when the cumulative alignment score falls off by the quantity X from its maximum achieved value, the cumulative score goes to zero or below due to the accumulation of one or more negative-scoring residue alignments, or the end of either sequence is reached.

In addition to calculating percent sequence identity, the BLAST algorithm also performs a statistical analysis of the similarity between two sequences. One measure of similarity provided by the BLAST algorithm is the smallest sum probability (P(N)), which provides an indication of the probability by which a match between two nucleotide or amino acid sequences would occur by chance. For example, a test nucleic acid sequence is considered similar to a reference sequence if the smallest sum probability in a comparison of the test nucleic acid sequence to the reference nucleic acid sequence is less than about 0.1, more preferably less than about 0.01, and most preferably less than about 0.001.

To obtain gapped alignments for comparison purposes, Gapped BLAST (in BLAST 2.0) can be utilized as described in Altschul et al. (1997) Nucleic Acids Res. 25:3389. Alternatively, PSI-BLAST (in BLAST 2.0) can be used to perform an iterated search that detects distant relationships between molecules. See Altschul et al., supra. When utilizing BLAST, Gapped BLAST, PSI-BLAST, the default parameters of the respective programs (e.g., BLASTN for nucleotide sequences, BLASTX for proteins) can be used. The BLASTN program (for nucleotide sequences) uses as defaults a wordlength (W) of 11, an expectation (E) of 10, a cutoff of 100, M=5, N=−4, and a comparison of both strands. For amino acid sequences, the BLASTP program uses as defaults a wordlength (W) of 3, an expectation (E) of 10, and the BLOSUM62 scoring matrix. See the world wide web at ncbi.nlm.nih.gov. Alignment may also be performed manually by visual inspection.

For purposes of the present invention, comparison of sequences for determination of percent sequence identity to another sequence may be made using the BlastN program (version 1.4.7 or later) with its default parameters or any equivalent program. By “equivalent program” is intended any sequence comparison program that, for any two sequences in question, generates an alignment having identical nucleotide or amino acid residue matches and an identical percent sequence identity when compared to the corresponding alignment generated by the preferred program.
(c) As used herein, “sequence identity” or “identity” in the context of two nucleic acid or polypeptide sequences makes reference to a specified percentage of residues in the two sequences that are the same when aligned for maximum correspondence over a specified comparison window, as measured by sequence comparison algorithms or by visual inspection. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity.” Means for making this adjustment are well known to those of skill in the art. Typically this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).
(d) As used herein, “percentage of sequence identity” means the value determined by comparing two optimally aligned sequences over a comparison window, wherein the portion of the sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.
(e)(i) The term “substantial identity” of sequences means that a polynucleotide comprises a sequence that has at least 70%, 71%, 72%, 73%, 74%, 75%, 76%, 77%, 78%, or 79%, at least 80%, 81%, 82%, 83%, 84%, 85%, 86%, 87%, 88%, or 89%, at least 90%, 91%, 92%, 93%, or 94%, and at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, at least 95%.

Another indication that nucleotide sequences are substantially identical is if two molecules hybridize to each other under stringent conditions (see below). Generally, stringent conditions are selected to be about 5° C. lower than the thermal melting point (Tm) for the specific sequence at a defined ionic strength and pH. However, stringent conditions encompass temperatures in the range of about 1° C. to about 20° C., depending upon the desired degree of stringency as otherwise qualified herein. Nucleic acids that do not hybridize to each other under stringent conditions are still substantially identical if the polypeptides they encode are substantially identical. This may occur, e.g., when a copy of a nucleic acid is created using the maximum codon degeneracy permitted by the genetic code. One indication that two nucleic acid sequences are substantially identical is when the polypeptide encoded by the first nucleic acid is immunologically cross reactive with the polypeptide encoded by the second nucleic acid.
(e)(ii) The term “substantial identity” in the context of a peptide indicates that a peptide comprises a sequence with at least 70%, 71%, 72%, 73%, 74%, 75%, 76%, 77%, 78%, or 79%, 80%, 81%, 82%, 83%, 84%, 85%, 86%, 87%, 88%, or 89%, at least 90%, 91%, 92%, 93%, or 94%, or 95%, 96%, 97%, 98% or 99%, sequence identity to the reference sequence over a specified comparison window. Optimal alignment is conducted using the homology alignment algorithm of Needleman and Wunsch, J. Mol. Biol. 48:443 (1970). An indication that two peptide sequences are substantially identical is that one peptide is immunologically reactive with antibodies raised against the second peptide. Thus, a peptide is substantially identical to a second peptide, for example, where the two peptides differ only by a conservative substitution.

For sequence comparison, typically one sequence acts as a reference sequence to which test sequences are compared. When using a sequence comparison algorithm, test and reference sequences are input into a computer, subsequence coordinates are designated if necessary, and sequence algorithm program parameters are designated. The sequence comparison algorithm then calculates the percent sequence identity for the test sequence(s) relative to the reference sequence, based on the designated program parameters.

As noted above, another indication that two nucleic acid sequences are substantially identical is that the two molecules hybridize to each other under stringent conditions. The phrase “hybridizing specifically to” refers to the binding, duplexing, or hybridizing of a molecule only to a particular nucleotide sequence under stringent conditions when that sequence is present in a complex mixture (e.g., total cellular) DNA or RNA. “Bind(s) substantially” refers to complementary hybridization between a probe nucleic acid and a target nucleic acid and embraces minor mismatches that can be accommodated by reducing the stringency of the hybridization media to achieve the desired detection of the target nucleic acid sequence.

“Stringent hybridization conditions” and “stringent hybridization wash conditions” in the context of nucleic acid hybridization experiments such as Southern and Northern hybridizations are sequence dependent, and are different under different environmental parameters. Longer sequences hybridize specifically at higher temperatures. The thermal melting point (Tm) is the temperature (under defined ionic strength and pH) at which 50% of the target sequence hybridizes to a perfectly matched probe. Specificity is typically the function of post-hybridization washes, the critical factors being the ionic strength and temperature of the final wash solution.

By “variant” polypeptide is intended a polypeptide derived from the native protein by deletion (so-called truncation) or addition of one or more amino acids to the N-terminal and/or C-terminal end of the native protein; deletion or addition of one or more amino acids at one or more sites in the native protein; or substitution of one or more amino acids at one or more sites in the native protein. Such variants may results form, for example, genetic polymorphism or from human manipulation. Methods for such manipulations are generally known in the art.

Thus, the polypeptides of the invention may be altered in various ways including amino acid substitutions, deletions, truncations, and insertions. Methods for such manipulations are generally known in the art. For example, amino acid sequence variants of the polypeptides can be prepared by mutations in the DNA. Methods for mutagenesis and nucleotide sequence alterations are well known in the art. See, for example, Kunkel (1985) Proc. Natl. Acad. Sci. USA 82:488; Kunkel et al. (1987) Meth. Enzymol. 154:367; U.S. Pat. No. 4,873,192; Walker and Gaastra (1983) Techniques in Mol. Biol. (MacMillan Publishing Co., and the references cited therein. Guidance as to appropriate amino acid substitutions that do not affect biological activity of the protein of interest may be found in the model of Dayhoff et al., Atlas of Protein Sequence and Structure (Natl. Biomed. Res. Found. 1978). Conservative substitutions, such as exchanging one amino acid with another having similar properties, are preferred.

Thus, the genes and nucleotide sequences of the invention include both the naturally occurring sequences as well as mutant forms. Likewise, the polypeptides of the invention encompass naturally occurring proteins as well as variations and modified forms thereof. Such variants will continue to possess the desired activity. In certain embodiments, the deletions, insertions, and substitutions of the polypeptide sequence encompassed herein may not produce radical changes in the characteristics of the polypeptide. However, when it is difficult to predict the exact effect of the substitution, deletion, or insertion in advance of doing so, one skilled in the art will appreciate that the effect will be evaluated by routine screening assays.

Individual substitutions deletions or additions that alter, add or delete a single amino acid or a small percentage of amino acids (typically less than 5%, more typically less than 1%) in an encoded sequence are “conservatively modified variations,” where the alterations result in the substitution of an amino acid with a chemically similar amino acid. Conservative substitution tables providing functionally similar amino acids are well known in the art. The following five groups each contain amino acids that are conservative substitutions for one another: Aliphatic: Glycine (G), Alanine (A), Valine (V), Leucine (L), Isoleucine (I); Aromatic: Phenylalanine (F), Tyrosine (Y), Tryptophan (W); Sulfur-containing: Methionine (M), Cysteine (C); Basic: Arginine (R), Lysine (K), Histidine (H); Acidic: Aspartic acid (D), Glutamic acid (E), Asparagine (N), Glutamine (Q). In addition, individual substitutions, deletions or additions which alter, add or delete a single amino acid or a small percentage of amino acids in an encoded sequence are also “conservatively modified variations.”

The term “transformation” refers to the transfer of a nucleic acid fragment into the genome of a host cell, resulting in genetically stable inheritance. Host cells containing the transformed nucleic acid fragments are referred to as “transgenic” cells, and organisms comprising transgenic cells are referred to as “transgenic organisms”.

“Transformed,” “transgenic,” “transduced”, and “recombinant” refer to a host cell or organism into which a heterologous nucleic acid molecule has been introduced. The nucleic acid molecule can be stably integrated into the genome generally known in the art and are disclosed in Sambrook and Russell, supra. See also Innis et al., PCR Protocols, Academic Press (1995); and Gelfand, PCR Strategies, Academic Press (1995); and Innis and Gelfand, PCR Methods Manual, Academic Press (1999). Known methods of PCR include, but are not limited to, methods using paired primers, nested primers, single specific primers, degenerate primers, gene-specific primers, vector-specific primers, partially mismatched primers, and the like. For example, “Transformed,” “transgenic,” and “transduced” cells have been through the transformation process and contain a foreign gene integrated into their chromosome. The term “untransformed” refers to normal cells that have not been through the transformation process.

“Genetically altered cells” denotes cells which have been modified by the introduction of recombinant or heterologous nucleic acids (e.g., one or more DNA constructs or their RNA counterparts) and further includes the progeny of such cells which retain part or all of such genetic modification.

The terms “treat” and “treatment” refer to both therapeutic treatment and prophylactic or preventative measures, wherein the object is to prevent or slow down (lessen) an undesired physiological change or disorder, such as the growth, development or spread of cancer. For purposes of this invention, beneficial or desired clinical results include, but are not limited to, alleviation of symptoms, diminishment of extent of disease, stabilized (i.e., not worsening) state of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those prone to have the condition or disorder or those in which the condition or disorder is to be prevented.

The phrase “therapeutically effective amount” means an amount of CSAN modified cells of the present invention that (i) treats the particular disease, condition, or disorder, (ii) attenuates, ameliorates, or eliminates one or more symptoms of the particular disease, condition, or disorder, or (iii) prevents or delays the onset of one or more symptoms of the particular disease, condition, or disorder described herein. In the case of cancer, the therapeutically effective amount of the CSAN modified cells may reduce the number of cancer cells; reduce the tumor size; inhibit (i.e., slow to some extent and preferably stop) cancer cell infiltration into peripheral organs; inhibit (i.e., slow to some extent and preferably stop) tumor metastasis; inhibit, to some extent, tumor growth; and/or relieve to some extent one or more of the symptoms associated with the cancer. To the extent the therapeutic agent may prevent growth and/or kill existing cancer cells, it may be cytostatic and/or cytotoxic. For cancer therapy, efficacy can be measured, for example, by assessing the time to disease progression (TTP) and/or determining the response rate (RR).

Certain embodiments of the invention provide a fusion protein or polypeptide as described herein.

Certain embodiments of the invention provide a conjugate comprising a polypeptide as described herein.

Certain embodiments of the invention provide a cell comprising a conjugate or a CSAN described herein.

Certain embodiments of the invention provide a nucleic acid encoding a polypeptide described herein.

Certain embodiments of the invention provide an expression cassette comprising a nucleic acid sequence described herein and a promoter operably linked to the nucleic acid. In certain embodiments, the promoter is a regulatable promoter. In certain embodiments, the promoter is a constitutive promoter. In certain embodiments, the expression cassette further comprises an expression control sequence (e.g., an enhancer) operably linked to the nucleic acid sequence. Expression control sequences and techniques for operably linking sequences together are well known in the art.

Certain embodiments of the invention provide a vector comprising an expression cassette described herein.

Certain embodiments of the invention provide a cell comprising a polypeptide described herein, a nucleic acid described herein, an expression cassette described herein, or a vector described herein.

The invention will now be illustrated by the following non-limiting Examples.

### Example 1 Engineering Biomimetic Trogocytosis with Farnesylated Chemically Self-Assembled Nanorings

It was studied in this Example that whether targeted farnesylated chemically self-assembled nanorings (f-CSANs) could serve as a biomimetic trogocytosis vehicle for engineering directional cargo transfer between cells; thus, allowing cell-cell interactions to be monitored and facilitating cell-cell communications. The membranes of sender cells were stably modified by hydrophobic insertion with the targeted f-CSANs, which were efficiently transferred to receiver cells expressing the appropriate receptors by endocytosis. CSAN-assisted cell-cell cargo transfer (C4T) was demonstrated to be receptor-specific, and dependent on direct cell-cell interactions, the rate of receptor internalization and the level of receptor expression. In addition, C4T was shown to facilitate cell-to-cell delivery of an apoptosis inducing drug, as wells as antisense oligonucleotides (ASO). Taken together, the C4T approach is a potentially versatile biomimetic trogocytosis platform that can be deployed as a macro-chemical biological tool for monitoring cell-cell interactions and engineering cell-cell communications.

### INTRODUCTION

Multicellular life and disease are dependent on the engagement of cells with each other, either for the development, maintenance and regeneration of tissues or identifying and removing diseased tissues¹⁻⁶. Cell-cell interactions between glial cells and neural cells are key to central nervous system (CNS) functions^{7,8}, while the contacts between T-cells and peripheral tissues are essential for defending against viral infections^{9,10}. Cell-based therapeutics have rapidly emerged and expanded as invaluable tools in translational medicine with a significant impact on several diverse fields, including tissue engineering, regenerative medicine, and immunotherapy¹¹⁻¹³ The potential to augment and modulate the effects of cells on other cells is of key interest to engineering synthetic biological processes. Consequently, recent approaches have begun to be developed for the monitoring and engineering of cell-cell interactions. Cell-cell interactions have been monitored by genetically engineering cells to take advantage of surface modifying non-discriminating chemical conjugation reactions or non-genetically by microdissection methods^{14,15}. Genetic cargo transfer based approaches have emerged that rely on the binding of engineered receptors on receiver cells to engineered fluorescent or chemically modified proteins fused to membrane spanning domains on sender cells^{16,17}. In this last approach, cargo transfer has also been used to deliver proteins and nucleic acids to the receiver cells from the sender cells¹⁶. Each of these approaches has proved versatile. Nevertheless, in each case genetic engineering of either the sender cell or receiver cell or both is required, which can be time-consuming, inefficient, and, in some cases, difficult, since not all cells are amenable to genetic modification¹⁸. Consequently, alternative methodologies that allow for the non-genetic modification of normal cells and the evaluation of their interactions with cells expressing a variety of natural or engineered receptors would be of value. In addition, the potential for modulation of the receiver cell biology through a non-diffusible modulating ligand from another cell, would expand and complement diffusible cell-cell communications approaches; thus, facilitating interrogation and control of cell-cell interactions both in healthy and diseased tissues.

To monitor and engineer cell-cell communications using non-genetic cargo transfer methodologies, the functional cargo should be displayed on the surface of the cells through membrane modification. Such modification is expected to be stable enough so that the cargo dissociation from the cell surface is negligible and the cargo transfer can occur only during the cell-cell interactions. On the other hand, the surface-anchored cargo should also exhibit some level of mobility so that the cell-cell communication events can efficiently trigger the cargo transfer by receptor recognition or other stimuli. Moreover, the cargo should be able to carry detectable signals or functional payloads for monitoring or manipulating cell-cell communications. To this end, the lapidated CSANs system as a non-genetic approach to engineer biomimetic trogocytosis, a natural intercellular material transfer process, for monitoring and engineering cell-cell communications was developed.

Farnesylated CSANs as a universal system to modify the mammalian cell surface for reversible cell-cell interactions has been reported¹⁹. The CSANs are oligomerized into predominantly octameric nanorings through the self-assembly of dimeric dihydrofolate reductase (DHFR²) fusion proteins by a chemical dimerizer, bis-methotrexate (bisMTX)²⁰⁻²² (FIG. 1a). The targeting fragments fused onto the N-terminus of the DHFR² proteins enable the CSANs to engage specific cellular receptors, while the C-terminal “CVIA” sequence of the proteins can be recognized and rapidly farnesylated by protein farnesyltransferase²³⁻³⁰. The consequent farnesylated DHFR² proteins can be easily self-assembled into farnesylated CSANs (f-CSANs) by incubation with bisMTX and used to efficiently modify mammalian cell membranes through hydrophobic interactions between the isoprenoid groups of the nanorings and the membrane phospholipids¹⁹ (FIG. 1b). f-CSANs were shown to be stably bound to cell surfaces for days (T_{1/2}>3 days) and direct modified cells to the desired target cells¹⁹. In this Example, it is reported that the f-CSANs on sender cell membranes undergo efficient CSAN-assisted cell-cell cargo transfer (C4T). C4T was found to be dependent on the internalization rate of the surface receptor, on the receiver cell, and the amount of the targeted receptor expressed on the receiver cell. In addition, by varying the concentration of the f-CSANs on the sender cells, induced interactions between the sender and receiver cells could be kept to a minimum, thus reducing the effect of the modification on natural or engineered interactions between the sender and receiver cells. Moreover, by incorporating azide-functionalized farnesyl analogs as bioconjugation handlesO, both an apoptosis inducing drug, MMAE, and antisense oligonucleotide targeting expression of the translation initiation factor, eIF4E, were able to undergo C4T transfer (Figure ic). Thus, the C4T approach was shown to be a versatile approach for demonstrating cell-cell interactions as well as engineering cell-cell communication.

### Experimental Methods

Expression plasmids and oligonucleotides.

gBlock Gene Fragments coding for the αEGFR-Fn3-DHFR²-CVIA, αHER²-afb-DHFR²-CVIA, αEpCAM-Fn3-DHFR²-CVIA, and αCD133-scFv-DHFR² fusion proteins were ordered from Integrated DNA Technologies (IDT) and cloned into the Novagen pET28a(+) vector (EMD Millipore, Cat: 69864-3) via NcoI and XhoI restriction sites. The gene fragment for the DHFR²-CVIA protein was generated via site-directed mutagenesis of the gene of αEpCAM-Fn3-DHFR²-CVIA protein using a New England Biolabs Q5 Site-Directed Mutagenesis Kit (Cat: E0554S). The ssDNAs containing DBCO functional group were ordered from Integrated DNA Technologies (IDT). The sequences of the protein constructs and ssDNAs are listed in Table A1.

### Protein Expression and Purification.

The αEGFR-Fn3-DHFR²-CVIA, αHER2-afb-DHFR²-CVIA, αEpCAM-Fn3-DHFR²-CVIA, αCD133-scFv-DHFR², and DHFR²-CVIA fusion proteins were produced in T7 Express Competent E. coli cells (New England Biolabs) using 0.5 mM IPTG at 37° C. for 3-6 h. The αEGFR-Fn3-DHFR²-CVIA and αEpCAM-Fn3-DHFR²-CVIA fusion proteins were purified from the soluble fractions of the cell lysate via immobilized metal affinity chromatography (IMAC) using the cobalt column (Thermo Fisher Scientific, Cat: 89964) according to previously reported methods (Wang, Y., et al., (2021), Chem. Sci. 12, 331-340); meanwhile, the αHER2-afb-DHFR²-CVIA and DHFR²-CVIA fusion proteins were purified from the soluble fractions of the cell lysate via methotrexate affinity chromatography and DEAE ion-exchange chromatography, also according to the previously reported methods (Wang, Y., et al., (2021), Chem. Sci. 12, 331-340). The αCD133-scFv-DHFR² protein was purified from the insoluble fractions of the cell lysate via previously reported denaturation and refolding procedures, followed by Q Sepharose Fast Flow anion exchange chromatography and SEC (Shen, J., et al., (2015), J. Am. Chem. Soc. 137, 10108-10111). Purified protein was analyzed by gel electrophoresis using NuPAGE Bis-Tris protein gels (Thermo Fisher Scientific, Cat: NP0321PK2). DTT (5 mM) was added tothe samples for gel electrophoresis. Yeast farnesyl transferase (yFTase) was expressed and purified following the previously reported procedures (Wang, Y., et al., (2021), Chem. Sci. 12, 331-340), Zhang, Y., et al. (2015), Bioconjug.Chem. 26, 2542-25530.

To prepare the fluorescein-labeled proteins, the protein of interest (10 μM) was incubated with a 10-fold molar excess of NHS-Fluorescein (Thermo Fisher Scientific, Cat: 46410) in PBS for 12 h at room temperature and then purified through buffer exchange with the Amicon Ultra-0.5 centrifugal filters (10 kDa cutoff, Millipore). The NHS-Fluorescein labeling of the proteins was confirmed by gel electrophoresis, followed by in-gel fluorescent scanning using the Typhoon FLA 9500 (GE Healthcare) and then the Coomassie brilliant blue staining. Gel images were processed in ImageJ.

### Cell Lines and Cell Culture.

The A431, MDA-MB-231, MDA-MB-453, SK-BR-3, HT29, RAW 264.7, HUVEC, Raji, and NK-92 cells were previously purchased from the American Type Culture Collection (ATCC). The J558L cells were obtained from Dr. Bruce Walcheck. The A431, MDA-MB-231, MDA-MB-453, SK-BR-3, and HT29 cells were transfected to express the nuclear-restricted mKate2 red fluorescent protein using the IncuCyte® NucLight Lentivirus Reagents (Sartorius, Cat: 4476) and following the manufacturer's protocol. The consequent red fluorescent cells were renamed with a suffix “R” (A431-R, MDA-MB-231-R, MDA-MB-453-R, SK-BR-3-R, and HT29-R).

A431-R, MDA-MD-231-R, RAW 264.7, and J558L cells were cultured in Dulbecco's Modified Eagle's Medium (DMEM) with 4.5 g/L glucose, L-glutamine, and supplemented with 10% fetal bovine serum (FBS), 100 U/mL penicillin, and 100 μg/mL streptomycin at 37° C. with 5.0% CO₂. MDA-MB-453-R and Raji cells were cultured in Roswell Park Memorial Institute (RPMI) medium with L-glutamine and supplemented with 10% FBS, 100 U/mL penicillin, and 100 μg/mL streptomycin at 37° C. with 5.0% CO₂. SK-BR-3-R and HT29-R cells were cultured in McCoy's 5A (Modified) Medium and supplemented with 10% FBS, 100 U/mL penicillin, and 100 μg/mL streptomycin at 37° C. with 5.0% CO₂. NK-92 cells were cultured in Minimum Essential Medium a and supplemented with 10% FBS, 100 U/mL penicillin, 1000 U/mL IL-2, and 100 μg/mL streptomycin at 37° C. with 5.0% CO₂. HUVEC cells were cultured using the EGM-2™ Endothelial Cell Growth Medium-2 BulletKit™ (Lonza, Cat: CC-3162).

Peripheral blood mononuclear cells (PBMCs) were purified from buffy coats of healthy donors blood samples using Ficoll-Hypaque density gradient centrifugation as previously described²² and cultured in ImmunoCult™-XF T Cell Expansion Medium supplemented with 30 U/ml IL-2, 100 U/mL penicillin, and 100 μg/mL streptomycin at 37° C. with 5.0% CO₂. The healthy donor blood samples (Donor 9) were purchased from Memorial Blood Centers, Saint Paul, MN. The CD8+ T cells were isolated using the Dynabeads™ CD8 Positive Isolation Kit (Invitrogen, Cat: 11333D) and cultured in ImmunoCult™-XF T Cell Expansion Medium supplemented with 30 U/ml IL-2, 100 U/mL penicillin, and 100 μg/mL streptomycin at 37° C. with 5.0% CO₂.

### Quantification of Cell Surface Receptors by Flow Cytometry.

The Bangs beads (Bangs Laboratories, Cat: 815A) were collected and resuspended in PBS, and then stained with the fluorescently labeled antibody that detects the corresponding cell surface receptor (anti-EGFR-BV421, Biolegend, Cat: 352911; anti-HER2-BV421, Biolegend, Cat: 324420; anti-EpCAM-AF647, Biolegend, Cat: 118211) at 4° C. for >30 min, followed by wash steps. 100,000 of the cancer cells were collected and stained with the corresponding antibody at 4° C. for >30 min, followed by wash steps. The cells and beads were analyzed by flow cytometry. The calibration curve was generated based on the MFI of the beads and the corresponding antibody binding capacity of the beads. The receptor number was calculated based on the MFI of the cells stained with the antibody and the previously generated calibration curve. The confirmation of the CD133 expression on HT29-R cells was carried out with a similar flow cytometry experiment, where the anti-CD133-PE antibody (Biolegend, Cat: 372803) was used to stain the HT29-R cells at 4° C. for >30 min, followed by wash steps and flow cytometry analysis.

### Preparation of Farnesylated Proteins and Protein Conjugates.

Farnesylation reactions were conducted following the previously reported methods. Specifically, a reaction cocktail (typically 500 uL) was prepared with the Dulbecco's phosphate-buffered saline (DPBS) buffer (Gibco, Cat: 14040141) containing MgCl₂ 222 (0.5 mM), ZnCl₂ (10 μM), DTT (5 mM), and the protein of interest (2.5 μM). The mixture was incubated on ice for 0.5 h and the reaction was initiated by the addition of FPP (7.5 μM) or C10-N₃-OPP (10 μM) with yFTase (200-400 nM) and allowed to proceed for 3-6 h in a 32° C. water bath. The prenylated protein was subsequently purified by buffer exchange (PBS) with an Amicon Ultra-0.5 centrifugal filter (10 kDa cutoff, Millipore) for cell surface modification or the following conjugation reactions.

For the preparation of protein-drug conjugates, the purified azide containing farnesylated proteins were incubated with a 10-fold molar excess of the DBCO-PEG4-VC-PAB-MMAE (ACES Pharma.) at room temperature in the dark for 12 h, followed by dialysis purification in PBS. Similarly, to prepare the protein-ssDNA conjugates, the azide containing farnesylated proteins were incubated with a 10-fold molar excess of the DBCO-functionalized ssDNA at room temperature in the dark for 12 h, followed by buffer exchange (PBS) with the Amicon Ultra-0.5 centrifugal filters (10 kDa cutoff, Millipore).

The proteins and protein conjugates were washed into ultrapure water with the Amicon Ultra-0.5 centrifugal filters (10 kDa cutoff, Millipore). 50 μL of each protein (5 μM) or the protein conjugates was characterized by LC-MS using an Orbitrap Elite Hybrid Mass Spectrometer. The data were further processed by the Thermo Scientific™ Protein Deconvolution software.

f-CSAN Formation and Characterization.

f-CSANs were formed by the addition of a 1.1-1.5-fold molar excess of the dimerizer, bisMTX, to a solution of the DHFR² fusion protein monomers (1-2 mL, unless specified otherwise). The oligomerization occurs within minutes after adding bisMTX. F-CSAN formation was characterized by dynamic light scattering and Cyro-TEM imaging. The hydrodynamic diameters of f-CSANs were measured by dynamic light scattering with an Anton Paar particle size analyzer (Litesizer 500) and presented as mean value±standard deviation of at least three measurements. The f-CSAN samples for cryo-TEM were prepared at 1 μM concentrations in PBS buffer. The f-CSAN solutions (2.5 μL) were applied to a lacey Formvar/carbon grid (Ted Pella, Inc.; Cat: 01883) in the humidified chamber of a Vitrobot Mark IV (FEI), blotted for 13 seconds, and plunged into liquid ethane for vitrification. Grids were imaged on a Tecnai Spirit G2 BioTWIN (FEI) equipped with an Eagle 2k CCD camera (FEI) under a high tension of 120 kV.

Assessing Cell Surface Labeling by f-CSANs by Flow Cytometry.

The binding specificity of the CSANs to the corresponding cellular receptors was studied by flow cytometry. A431-R cells were chosen as the EGFR⁺ cell line; SK-BR-3-R cells were chosen as the HER2⁺ cell line; HT-29-R cells were chosen as the EpCAM⁺ cell line; HT29-R cells were chosen as the CD133⁺ cell line. The cells were harvested and washed with DPBS buffer (Gibco, Cat: 14190144), and aliquots of 105 cells were then resuspended in 100 μL of DPBS solutions containing 0.5 μM of fluorescein-labeled CSANs and incubated for 1 h at 4° C. The cells were then pelleted, washed, and resuspended in 0.5 mL of cold DPBS and analyzed using an LSR II flow cytometer (BD Biosciences) at the University Flow Cytometry Resource (UFCR). For the cell surface modification with farnesylated CSANs, the sender cells were collected from cell culture, pelleted at 350 g for 5 min, and washed with 1 mL DPBS. Aliquots of 10⁵ cells were then incubated in 100 μL of DPBS solutions containing the desired concentrations of fluorescein-labeled farnesylated CSANs for at least 1 h at room temperature with rotation and washed twice with 1 mL cold DPBS to remove unbound CSANs. The modified cells were then resuspended in 0.5 mL of cold DPBS and analyzed using an LSR II flow cytometer (BD Biosciences) at the University Flow Cytometry Resource (UFCR).

Imaging of Surface-Bound f-CSANs by Fluorescent Microscopy

10⁵ Raji cells were harvested and washed with DPBS, followed by modification with the fluorescein-labeled αEGFR-Fn3-Far CSANs (1 μM) for at least 1 h at room temperature with rotation and washed twice with 1 mL cold DPBS to remove unbound CSANs. The cells were then transferred to the 35 mm glass coverslip bottom dish (ibidi, cat: 81158) and imaged by the Nikon Ti-E microscope with an ibidi Stage Top Incubation System.

Flow Cytometry Study of Non-Specific Intercellular Transfer of f-CSANs

The αEGFR-Fn3-DHFR²-CVIA protein was non-specifically labeled with DyLight™ 650 NHS Ester (Thermo Scientific, Cat: 62266) in PBS according to the manufacturer's protocol. The fluorescently labeled αEGFR-Fn3-DHFR²-CVIA (2 μM) was farnesylated following previously described methods, and the αEGFR-Fn3-DHFR²-Far protein was oligomerized to form CSANs. 4×10⁵ Raji cells were collected and stained with CFSE (Thermo Fisher Scientific, Cat: C34554) according to the manufacturer's protocol. Meanwhile, 6×10⁵ Raji cells were collected and modified with the DyLight™ 650-labeled αEGFR-Fn3-Far CSANs at room temperature with rotation for 1 hour, followed by wash steps with DPBS. The CSAN-modified Raji cells were mixed with the CFSE-stained Raji cells at a 6:4 ratio and were divided into 9 aliquots; each aliquot was incubated in 1 mL of the culture medium. 3 aliquots were analyzed by flow cytometry, and the other 6 aliquots of the cells were returned into the cell culture in a 6-well plate for 24-48 h in the incubator. At 24 h intervals, 3 aliquots of the cells were taken out for flow cytometry analysis. The media was refreshed every 24 h. An LSR II flow cytometer was used for the flow cytometry analysis, and the CSAN⁺/CFSE⁺ (DyLight 650⁺/CFSE⁺) population was quantified as the indicator of non-specific intercellular transfer of the αEGFR-Fn3-Far CSANs.

Flow Cytometry Study of the Cell-Cell f-CSAN Transfer Between Raji Cells and A431-R Cells.

Raji cells (6×10⁴ per sample) were collected and modified with the fluorescein-labeled αEGFR-Fn3-Far CSANs or fluorescein-labeled DHFR²-Far CSANs (1 μM) respectively at room temperature with rotation for 1 hour, followed by wash steps with DPBS. The CSAN-modified Raji cells were mixed with the A431-R cells at a 6:4 ratio and co-cultured in Eppendorf tubes with rotation at 37° C. (if not otherwise specified). The cells are then analyzed by an LSR II flow cytometer and the CSAN⁺/mKate⁺ (FITC⁺/mKate⁺) cell population was quantified as the indicator of intercellular transfer of CSANs from Raji cells to A431-R cells. Unless otherwise stated, experiments were conducted in triplicate and data are presented as the mean±standard deviation of three independent trials.

For the transwell assay of the cell-cell CSAN transfer study, Raji cells were collected and modified with the fluorescein-labeled αEGFR-Fn3-Far CSANs (1 μM), followed by wash steps with DPBS. The A431-R cells were collected, and one group of the cells were co-cultured together with the CSAN-modified Raji cells at a 1:1 ratio, while for the other group, the transwell inserts were used to separate the A431-R cells and the CSAN-modified cells. The cells were co-cultured at 37° C. for 30 min. Then the cells were analyzed by flow cytometry. The A431-R cells were gated out and the fluorescence of the fluorescein-labeled CSANs were quantified. All experiments were conducted in triplicate, and data are presented as the mean f standard deviation of three independent trials.

For the competition binding assay of the cell-cell CSAN transfer study, the Raji cells were collected and modified with the fluorescein-labeled αEGFR-Fn3-Far CSANs (1 μM), followed by wash steps with DPBS. A431-R cells were collected and incubated with different concentrations of unfarnesylated αEGFR-Fn3 CSANs (0-5000 nM) for 10 min, and then were co-cultured in Eppendorf tubes with the CSAN-modified Raji cells at a 6:4 ratio with rotation at 37° C. for 30 min, followed by flow cytometry analysis. All experiments were conducted in triplicate and data are presented as the mean±standard deviation of three independent trials.

To study the cell-cell CSAN transfer at a low sender-to-receiver ratio, the CSAN-modified Raji cells were co-cultured with A431-R cells at a 1:9 ratio with rotation at 37° C. for 1 h, followed by flow cytometry analysis. In addition, to study the cell-cell CSAN transfer at different temperatures, the CSAN-modified Raji cells were co-cultured with A431-R cells at a 6:4 ratio at 37° C. or 4° C. for 30 min, followed by flow cytometry analysis. All experiments were conducted in triplicates and data are presented as the mean±standard deviation of three independent trials.

Flow Cytometry Assessment of the f-CSAN Mediated Cell-Cell Interactions During the Intercellular CSAN Transfer.

Raji cells were collected, stained by the Hoechst dye (Invitrogen, Cat: H3570), and modified with the fluorescein-labeled αEGFR-Fn3-Far CSANs (0-2.5 μM) as previously described, followed by wash steps with DPBS. A431-R cells were collected and were co-cultured with the CSAN-modified Raji cells at a 1:1 ratio in tubes with rotation at 37° C. for 1 h, followed by flow cytometry analysis. The Hoechst⁺/mKate⁺ population was quantified to indicate cell-cell interactions, and the FITC⁺/mKate⁺ population was quantified to indicate cell-cell CSAN transfer. All experiments were conducted in triplicates and data are presented as the mean±standard deviation of three independent trials.

Determination of the Kinetics Study of the Cell-Cell f-CSAN Transfer.

The fluorescein-labeled farnesylated CSANs were prepared following the previously described methods. The Raji cells were collected and modified with the fluorescein-labeled farnesylated CSANs as previously described, followed by wash steps with DPBS. The CSAN-modified Raji cells were co-cultured with the corresponding mKate-expressing receiver cells at a 6:4 ratio with rotation at 37° C. for different times, followed by flow cytometry analysis. All experiments were conducted in triplicates and data are presented as the mean±standard deviation of three independent trials.

### Recording Cell-Cell Interactions by the C4T Approach Using Flow Cytometry

The sender cells were collected and modified with the fluorescein-labeled f-CSANs (1 μM) at room temperature with rotation for 1 hour, followed by wash steps with DPBS. The receiver cells were collected and co-cultured with the f-CSAN-modified sender cells in the tubes at a 6:4 ratio with rotation at 37° C. for 30 min, followed by the flow cytometry analysis. The mKate⁺ receiver cells were gated out and the fluorescence of the fluorescein-labeled f-CSANs was quantified. All experiments were conducted in triplicates and data are presented as the mean±standard deviation of three independent trials.

### Recording Cell-Cell Interactions by the C4T Approach Using Fluorescent Microscopy

1.5×10⁵ mKate-expressing target receiver cells were plated in the 35 mm glass coverslip bottom dish (ibidi, cat: 81158) one day prior to the imaging experiment. 10⁵ sender cells were collected and modified with the fluorescein-labeled f-CSANs at room temperature with rotation for 1 hour, followed by wash steps with DPBS. The f-CSAN modified sender cells were added to the cell culture dish of the mKate-expressing cells at 37° C. and then imaged by the Nikon Ti-E microscope with an Ibidi Stage Top Incubation System.

### Flow Cytometry and Imaging Study of the Interaction-Dependent Delivery of MMAE by the C4T Approach.

To study the interaction-dependent delivery of MMAE by the C4T approach using flow cytometry, the αEGFR-Fn3-DHFR2-MMAE was labeled with NHS-Fluorescein and oligomerized with the αEGFR-Fn3-DHFR2-Far protein in a 1:3 ratio to form the hybrid αEGFR-Fn3-Far-MMAE CSANs. The Raji cells were collected and modified with the hybrid αEGFR-Fn3-Far-MMAE CSANs (2 μM) at room temperature with rotation for 1 hour, followed by wash steps with DPBS. The CSAN-modified Raji cells were co-cultured with A431-R cells at a 6:4 ratio with rotation at 37° C. or 4° C. for 1 hour, followed by flow cytometry analysis. For the competition binding control, the A431-R cells were pre-incubated with 5 μM of unfarnesylated αEGFR-Fn3 CSANs for 10 min before being co-cultured with the CSAN-modified Raji cells. The cell-cell transfer of the hybrid αEGFR-Fn3-Far-MMAE CSANs from Raji cells to A431-R cells was also imaged by fluorescent microscope following previously described methods used for recording cell-cell interactions.

### Cytotoxicity Study of the Interaction-Dependent Delivery of MMAE by the C4T Approach.

2,500 of A431-R, MDA-MB-231-R, or MDA-MB-453-R cells were plated in the 96-well plates one day before the treatment. The hybrid αEGFR-Fn3-Far-MMAE CSANs were formed by oligomerizing αEGFR-Fn3-DHFR²-Far and αEGFR-Fn3-DHFR²-MMAE in a 3:1 ratio, while the control hybrid DHFR²-MMAE CSANs were formed by oligomerizing DHFR²-Far and DHFR²-MMAE in a 3:1 ratio. The Raji cells were collected and modified with the CSANs (2 μM) at room temperature with rotation for 1 hour, followed by wash steps with DPBS. The CSAN-modified Raji cells were co-cultured with the target cancer cells at a 3:1 ratio in the plate at 37° C. for 2 h. Then the Raji cells were removed by the medium exchange. The cancer cells were returned to the IncuCyte and cultured for 4 days to quantify cell viability.

### Assessment of the Delivery of Oligonucleotides by the C4T Approach.

To study the interaction-dependent delivery of ssDNA by the C4T approach using flow cytometry, the αEGFR-Fn3-ssDNA-AF488 protein conjugate was oligomerized with the αEGFR-Fn3-DHFR²-Far protein in a 1:3 ratio to form the hybrid αEGFR-Fn3-Far-ssDNA-AF488 CSANs. The Raji cells were collected and modified with the hybrid αEGFR-Fn3-Far-ssDNA-AF488 CSANs (2 μM) at room temperature with rotation for 1 hour, followed by wash steps with DPBS. The CSAN-modified Raji cells were co-cultured with A431-R cells at a 6:4 ratio with rotation at 37° C. or 4° C. for 1 hour, followed by flow cytometry analysis. For the competition binding control, the A431-R cells were pre-incubated with 5 μM of unfarnesylated αEGFR-Fn3 CSANs for 10 min before being co-cultured with the CSAN-modified Raji cells.

To study the specific knockdown of the targeted protein by the C4T approach, several protein-ssDNA conjugates were constructed as previously described, which include the αEGFR-Fn3-DHFR²-KDssDNA with an antisense phosphorothioate ssDNA targeting eIF4E, the αEGFR-Fn3-DHFR²-CTRLssDNA with a control ssDNA, and the non-targeting DHFR²-KDssDNA with the anti-eIF4E phosphorothioate ssDNA. The αEGFR-Fn3-DHFR²-KDssDNA was oligomerized with the αEGFR-Fn3-DHFR²-Far protein in a 1:3 ratio to form the hybrid αEGFR-Fn3-Far-KDssDNA CSANs. Meanwhile, the non-targeting hybrid DHFR²-Far-KDssDNA CSANs were formed by oligomerizing DHFR²-KDssDNA and DHFR2-Far in a 1:3 ratio, and the hybrid αEGFR-Fn3-Far-CTRLssDNA CSANs were formed by oligomerizing αEGFR-Fn3-DHFR2-CTRLssDNA and αEGFR-Fn3-DHFR²-Far protein in a 1:3 ratio. 5×10⁴ MDA-MB-231 cells were plated in each well of the 24-well plate one day prior to the co-culture experiment. Raji cells were collected and modified with the hybrid CSANs (2 μM) at room temperature with rotation for 1 hour, followed by wash steps with DPBS. The CSAN-modified Raji cells were then co-cultured with MDA-MB-231 cells at a 1:1 ratio at 37° C. for two days and removed from the co-culture through the medium exchange. The MDA-MB-231 cells were then collected, followed by cell lysis with RIPA buffer (Pierce, Cat: 89900) containing complete protease inhibitor cocktail (Roche, Cat: 11697498001). The cell lysate samples were collected and normalized to the same concentration, and an equivalent amount of protein lysate (12-15 μg) was electrophoresed on a 4-12% NuPAGE gradient gel and transferred onto low fluorescent polyvinylidene difluoride membranes (Bio-Rad, Cat: 1704274). Immunoblotting was performed with primary antibodies followed by secondary antibodies with the indicated dilutions: anti-eIF4E, 1:10,000 (R&D Systems, Cat: MAB3228); anti-mouse HRP, 1:1,000 (Invitrogen, Cat: A16072); anti-β-actin, 1:2,000 (Millipore Sigma, Cat: A1978); anti-mouse Alexa Fluor 680, 1;1,000 (Invitrogen, Cat: A32729). When using horseradish peroxidase-conjugated antibodies, West Femto Maximum Sensitivity Substrate (Thermo Scientific, Cat: 34095) was added to the membranes before imaging on an Odyssey Fc Imaging system (Li-Cor). Band intensity was quantified with ImageJ 1.53k.

### Statistical Information

Data analysis and data visualization were performed in GraphPad Prism8. Information about error bars, statistical tests, and n values are reported in each figure legend. Unless otherwise stated, experiments were conducted in triplicates, and data are presented as the mean±standard deviation of three independent trials. In the CSAN transfer kinetics study, the equations of the linear regression lines were generated for the calculation of the T50% values (for the kinetics study with A431-R cells, the datapoints within the first 10 minutes were used for linear regression analysis), and the slopes of the linear regression lines are statistically different. Differences between means were compared using the unpaired two-tailed Student's t-tests, and a P-value <0.05 is denoted in graphics with an (*), P<0.01 is denoted with (**), P<0.001 is denoted with (***), and P<0.0001 is denoted with (****).

### Results

Preparation of the CSANs that Target Cell Surface Receptors.

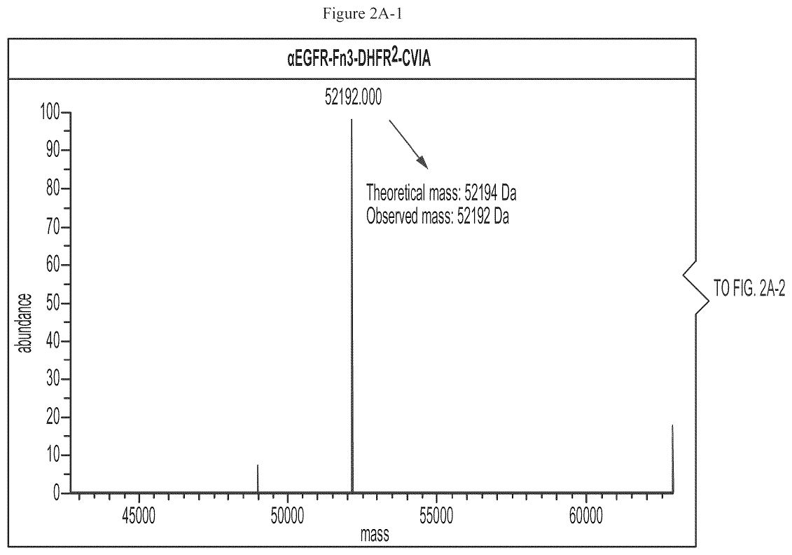

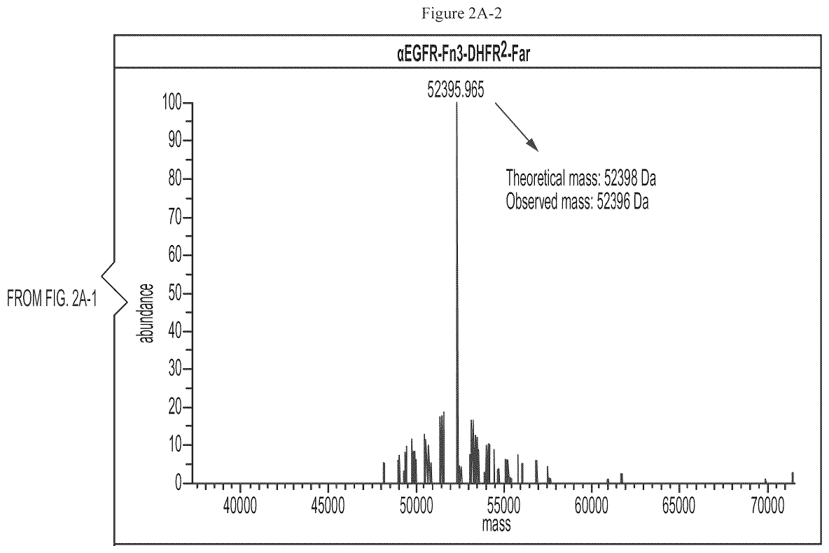

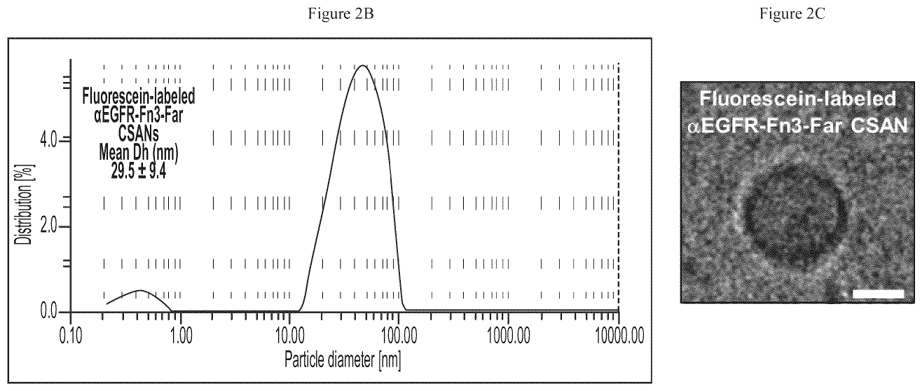

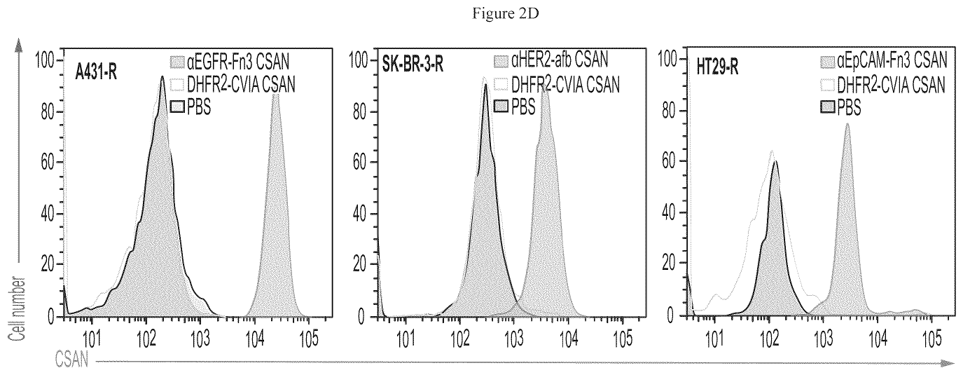

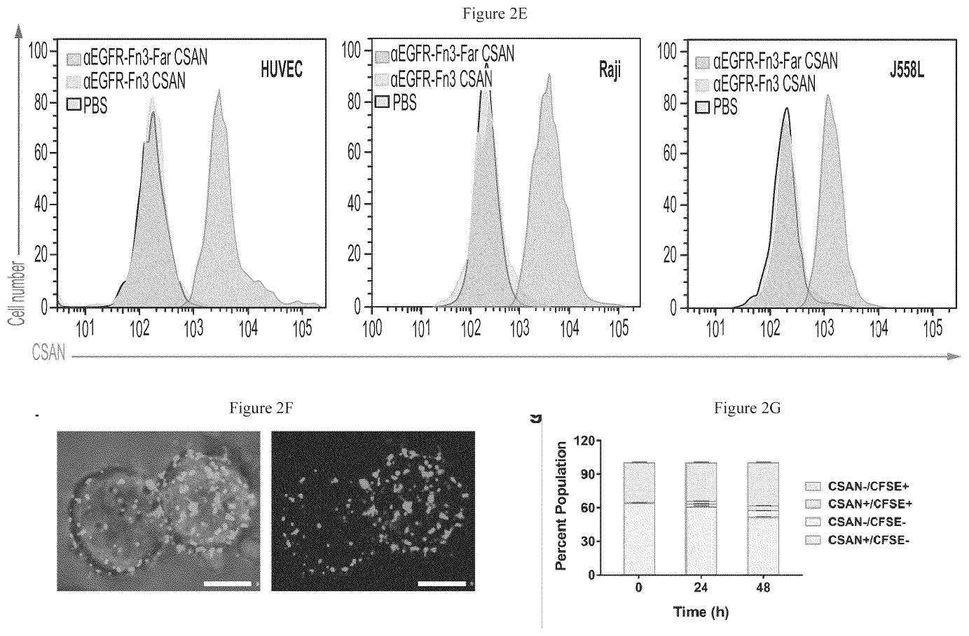

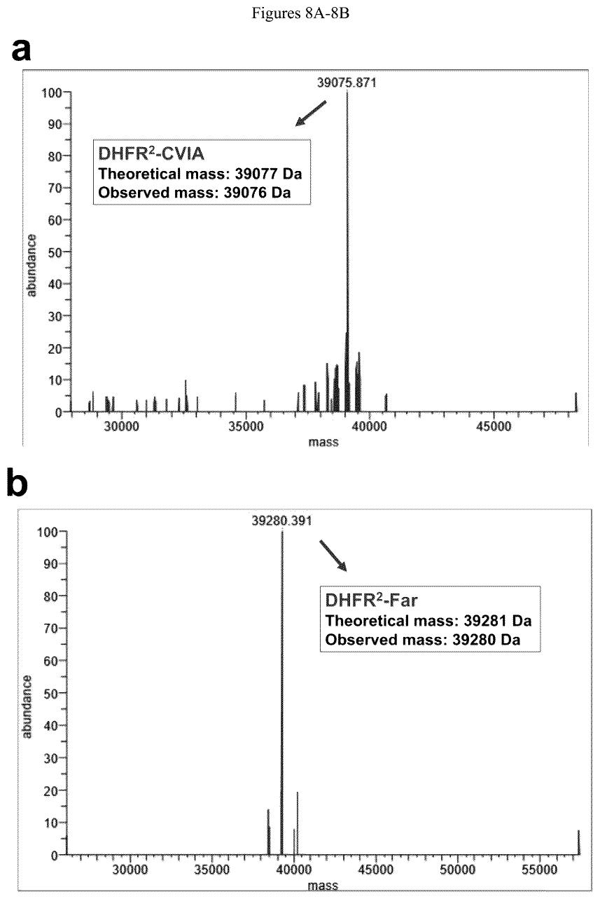

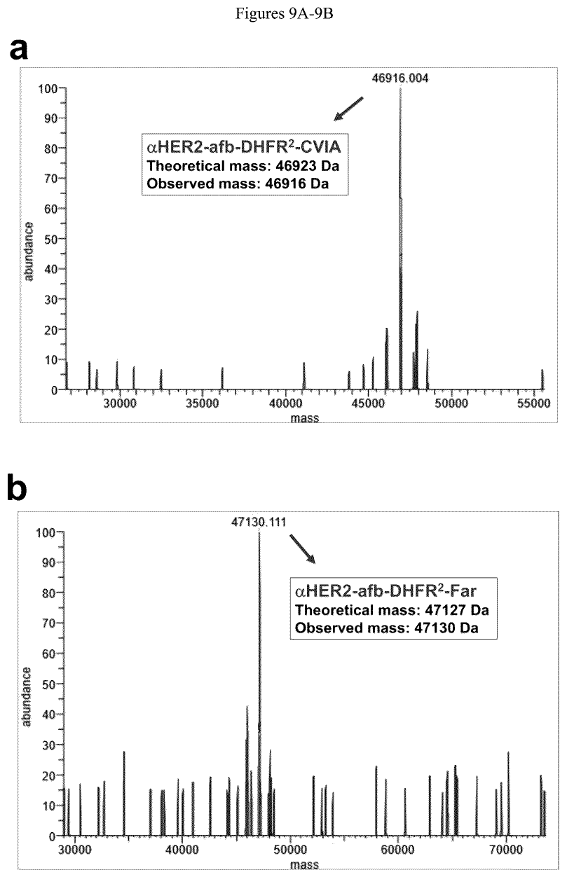

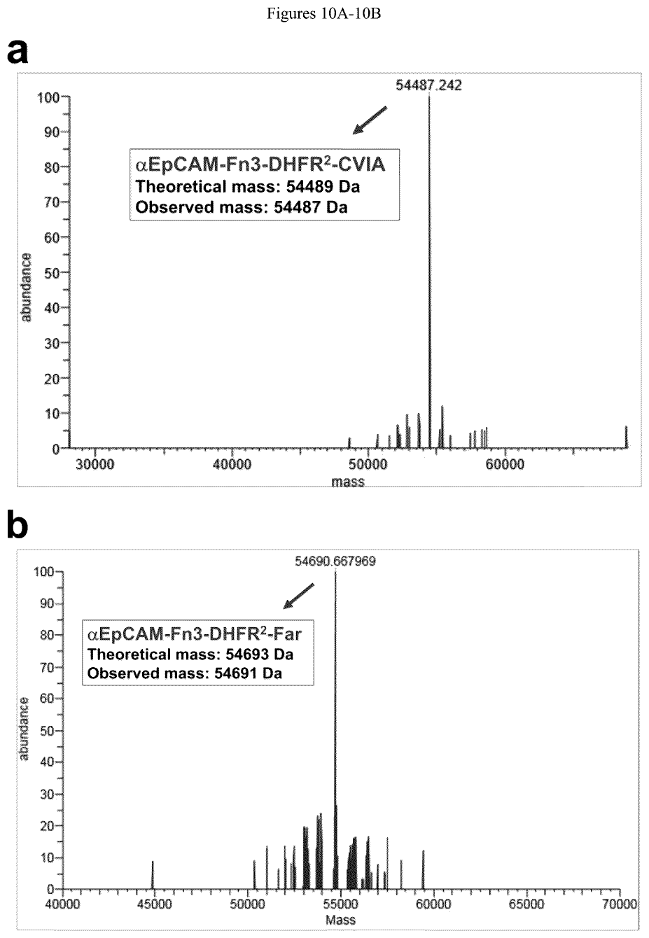

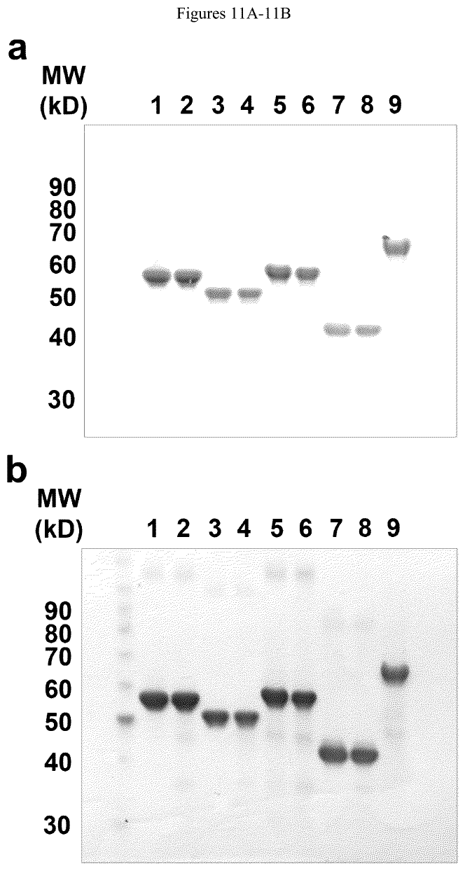

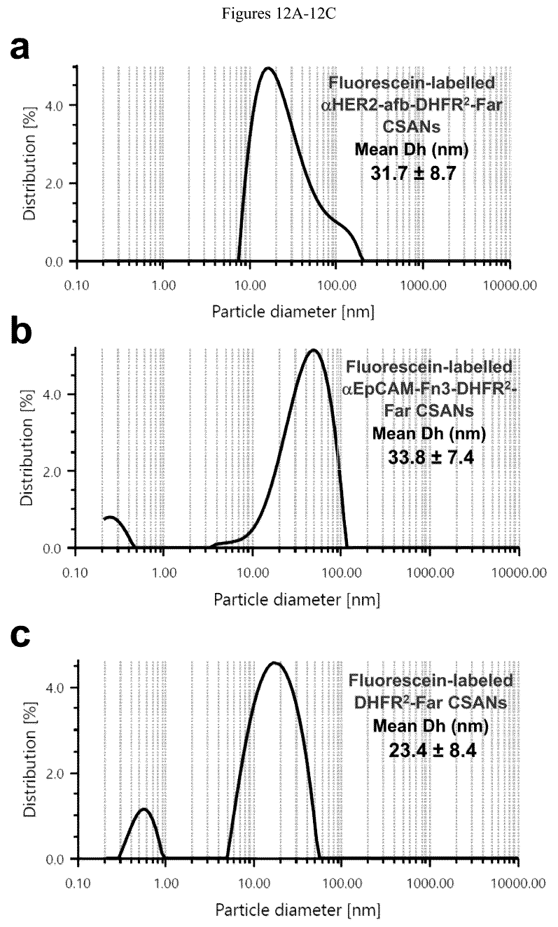

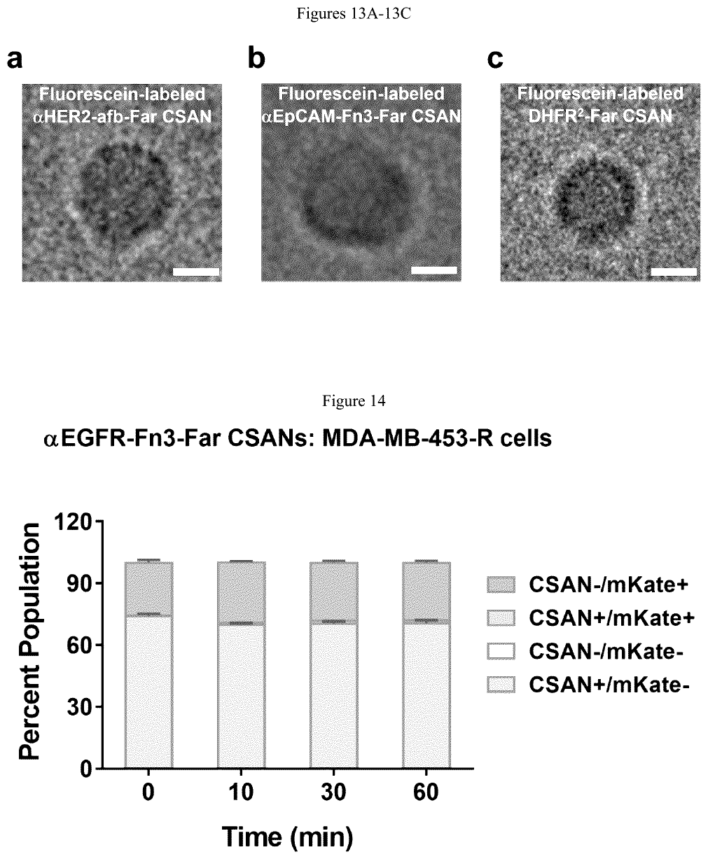

Our lab has previously developed multiple DHFR² fusion protein constructs that target a variety of cancer-specific antigens, including epidermal growth factor receptor (EGFR), human epidermal growth factor receptor 2 (HER2) and epithelial cell adhesion molecule (EpCAM). The EGFR-targeting DHFR² fusion protein (αEGFR-Fn3-DHFR²-CVIA) and the EpCAM-targeting protein (αEpCAM-Fn3-DHFR²-CVIA) contain the targeting domain that was previously generated based on the human tenth type III fibronectin (Fn3) scaffold and a C-terminal CVIA sequence, which is a substrate for farnesyltransferase³²⁻³⁵. The HER2-targeting DHFR² fusion protein (αHER2-afb-DHFR²-CVIA) carries an affibody-based (afb) targeting domain³⁶, while the CD133-targeting DHFR² fusion protein (αCD133-scFv-DHFR²) was prepared with an anti-CD133 scFv as the targeting element³⁷. As a control and for formation of hybrid f-CSANs, a non-targeting DHFR² fusion protein (DHFR²-CVIA) was also prepared. All the DHFR² fusion proteins were expressed in E coli, followed by purification and then farnesylated by farnesyltransferase to prepare the farnesylated DHFR² proteins (αEGFR-Fn3-DHFR²-Far, αHER2-afb-DHFR²-Far, αEpCAM-Fn3-DHFR²-Far, and DHFR²-Far), followed by characterization with LC-MS (FIGS. 2A and 8-10). The DHFR² fusion proteins were also non-specifically labeled with NHS-fluorescein as the fluorophore for the detection of CSANs on the surface of cells (FIG. 11). The fluorescein-labeled farnesylated DHFR² proteins were then self-assembled into the corresponding CSANs by bisMTX. The hydrodynamic diameters of the nanorings were characterized by dynamic light scattering (DLS) and the sizes of the fluorescein-labeled farnesylated CSANs were shown to be approximately 30 nm (FIGS. 2B and 12). Cryo-transmission electron microscopy (cryo-TEM) imaging analysis further confirmed the formation of nanoring structures and revealed that the sizes of the nanorings were consistent with the DLS analysis (FIGS. 2C and 13). Moreover, the specificity of the CSANs for their target receptors was confirmed by flow cytometry demonstrating that the unfarnesylated receptor-targeting CSANs selectively bound to the receptor expressing target cells, while the non-targeting DHFR²-CVIA CSANs exhibited no observable binding. (FIG. 2D).

### Farnesylated CSANs Serve as a Universal System for Cell Surface Modifications.

Previously, it has been demonstrated that prenylated CSANs are able to universally modify the mammalian cell surface by thermodynamically favored hydrophobic insertion¹⁹. f-CSANs were shown to efficiently modify primary human endothelial cells (HUVECs), human lymphoblastoid cells (Raji), and mouse myeloma cells (J558L), confirming their ability to modify cell surfaces across species and tissue/cell types (FIG. 2E). Consistent with our previous observation that prenylated CSANs preferentially insert into lipid rafts, imaging of the modified cell surfaces by fluorescent microscopy revealed the characteristic semi-discrete localization of the f-CSANs on the cell membranes¹⁹ (FIG. 2F).

Previously, due to their multivalency, f-CSANs were shown to stably modify cell surfaces (T_{1/2}>3 days)¹⁹. To characterize the potential non-specific transfer of the f-CSANs to adjacent unmodified cells, Raji cells were modified with f-CSANs that had been fluorescently labeled with DyLight-650. The CSAN-modified Raji cells were mixed with unmodified CFSE-labeled Raji cells, followed by co-culturing for 0-48 h. The amount of f-CSANs transfer was assessed by determining the percentage of CSAN⁺/CFSE⁺ proportion by flow cytometry. Over a 48-h period less than 5% of the Raji cells were shown to be CSAN⁺/CFSE⁺; thus, negligible non-specific cell-cell transfer of the f-CSANs was observed (FIG. 2G).

Farnesylated CSANs Specifically Transfer from the Sender Cells to the Receiver Cells During Cell-Cell Interactions.

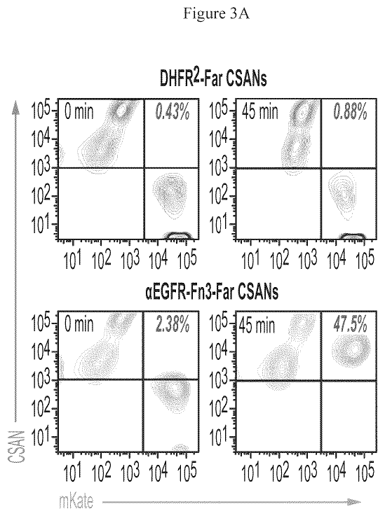

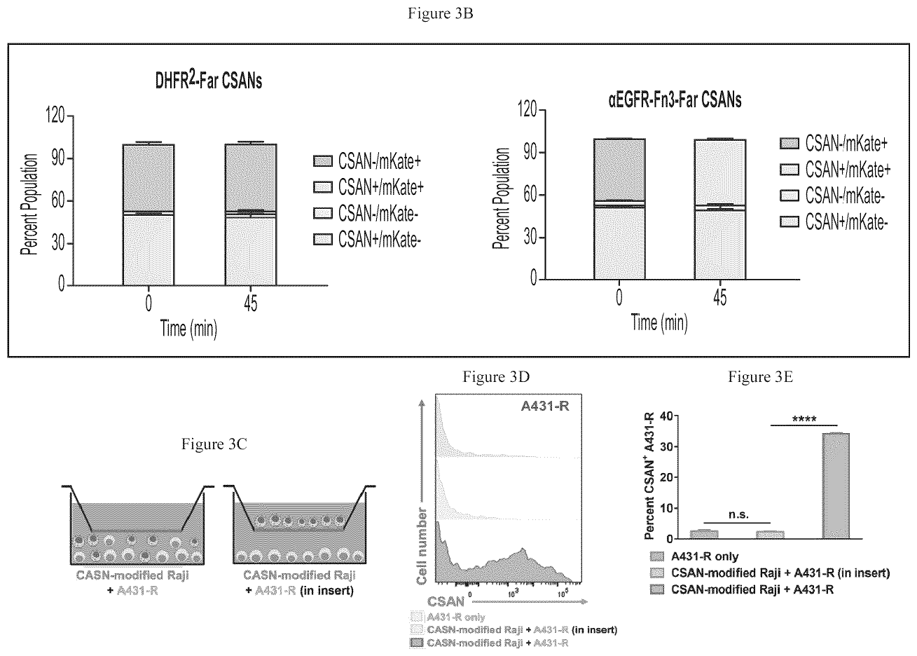

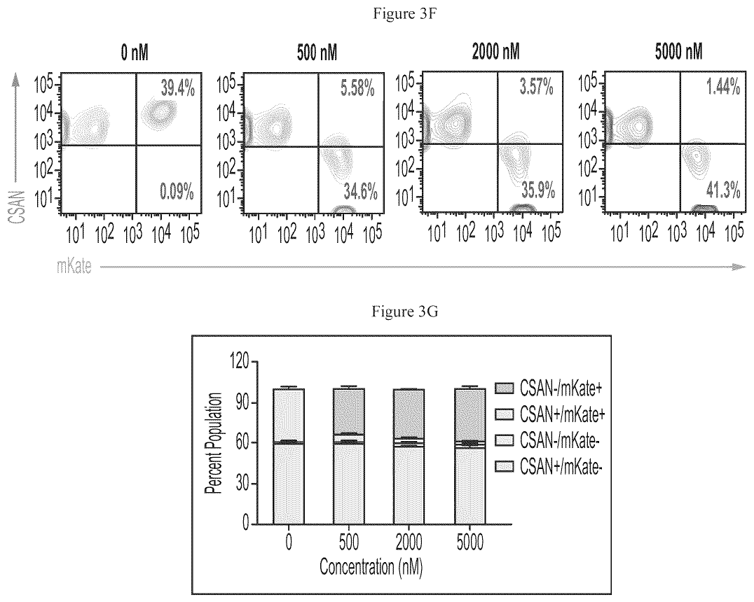

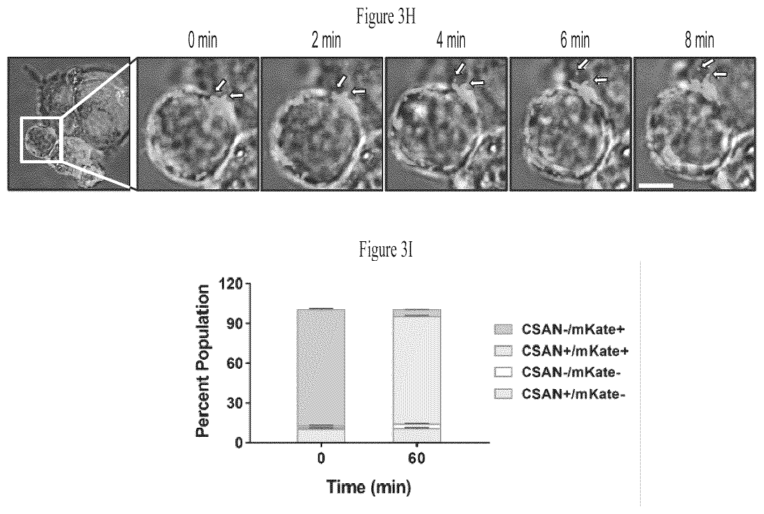

To investigate if the farnesylated CSANs on the cell surface can transfer to the target cells upon cell-cell interactions, Raji cells were modified with either fluorescein-labeled αEGFR-Fn3-Far CSANs or fluorescein-labeled non-targeting DHFR²-Far CSANs. The sender αEGFR-Fn3-Far-CSAN modified Raji cells were then co-cultured with the receiver EGFR⁺ A431-R cells that had been transduced to express the red fluorescent mKate protein as the marker. The co-culturing was carried out at 37° C. with rotation to enhance 3D interactions between the cells. After being co-cultured for 45 minutes, the cells were subjected to flow cytometry analysis, and the percentage of the CSAN⁺/mKate⁺ proportion was quantified to determine the extent of cell-cell transfer of the αEGFR-Fn3-Far CSANs to the A431-R cells. Within 45 minutes, 100% of the A431-R cells displayed the fluorescein signal of the αEGFR-Fn3-Far CSANs, indicating that rapid transfer of the αEGFR-Fn3-Far CSANs from modified Raji cells to the EGFR⁺ A431-R cells had occurred. No detectable f-CSAN transfer to the A431-R cells was observed with Raji cells modified with non-targeted f-CSANs (FIG. 3A, 3B).

To probe whether direct cell-cell contact is a requirement for intercellular CSAN transfer, a transwell assay with the transwell insert containing a porous membrane was carried out in which Raji cells modified with αEGFR-Fn3-Far CSANs were placed in the upper compartment, while A431-R cells were cultured in the lower compartment (FIG. 3C). As shown by flow cytometry data, for the A431-R receiver cells co-cultured together with the αEGFR-Fn3-Far-CSAN modified Raji cells, a significant increase in fluorescein intensity was observed in 30 min as expected. In contrast, when placed in the transwells, no significant fluorescein intensity was observed to have been transferred from the αEGFR-Fn3-Far-CSAN modified Raji cells to the A431-R cells (FIG. 3D, 3E).

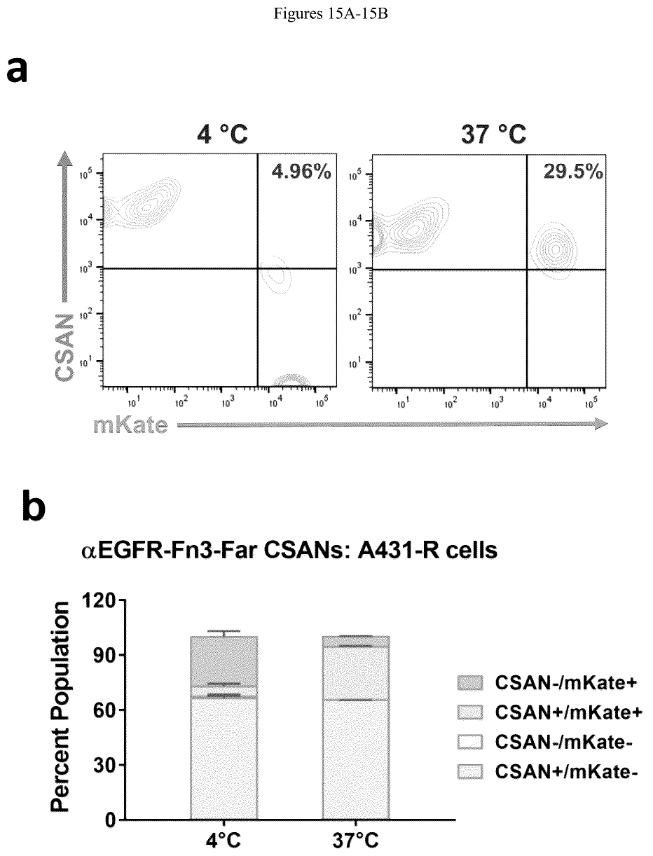

To verify the specificity of the transfer, an increasing amount of non-farnesylated αEGFR-Fn3 CSANs (0-5,000 nM) was added to the A431-R cells and shown to block transfer of αEGFR-Fn3-Far CSANs from the CSAN-modified Raji cells (FIG. 3F, 3G). Moreover, no significant transfer was observed from the αEGFR-Fn3-Far-CSAN modified Raji cells to EGFR-MDA-MB-453-R cells (FIG. 14). Taken together, these results are consistent with the specific transfer of αEGFR-Fn3-Far CSANs from sender cells to EGFR⁺ receiver cells.

Rapid transfer within minutes could be observed by live-cell imaging microscopy from sender cells modified with the fluorescein labeled αEGFR-Fn3-Far CSANs to EGFR⁺ A431-R cells, followed by internalization (FIG. 3h). Taken together, these results are consistent with the high stability of the f-CSANs on cell membranes and the need for direct physical contact between the sender cells and receiver cells for f-CSAN cargo transfer.

Cell-Cell Cargo Transfer and Interactions are Dependent on Sender Cell to Receiver Cell Ratio and f-CSANs Concentration.

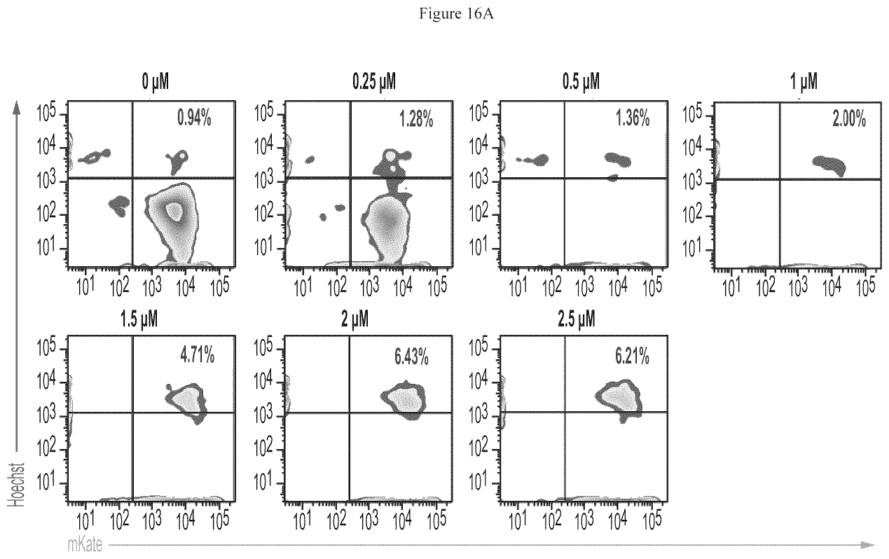

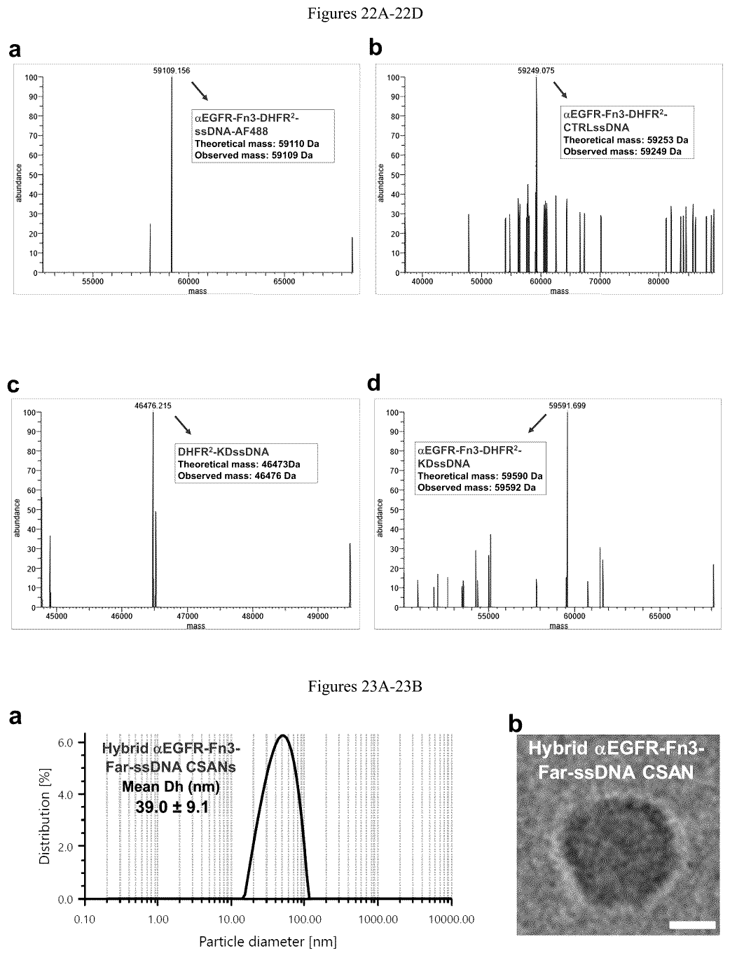

Given that, even under conditions in which transfer of the f-CSANs has resulted in 100% of the receiver cells being labelled, greater than 95% of the sender cells remain labelled with f-CSANs; thus, sender cells could in principle label multiple cells. To assess the ability of sender cells to transfer f-CSANs to multiple receiver cells, Raji cells were modified with αEGFR-Fn3-Far CSANs and co-cultured with A431-R cells in a 1:9 sender: receiver ratio, followed by flow cytometry analysis. After co-culture for one hour, CSANs were transferred from the sender cells to nearly 100% of the receiver cells without a significant decrease in the percentage of CSAN⁺ sender cells (FIG. 3I, 15), indicating that the sender cells were able to transfer the αEGFR-Fn3-Far CSANs to multiple receiver cells. In addition, when the amount of cargo transfer over time was compared at 4° C. and 37° C., significantly less (6-fold) αEGFR-Fn3-Far CSANs were found to have been transferred to the receiver cells at the lower temperature, indicating that the targeted f-CSANs transfer is at least partially dependent on EGFR-based endocytosis (FIG. 15), and the fluorescent microscopy imaging also demonstrated the binding of the targeting CSANs to the surface receptors can lead to endocytosis (FIG. 23).

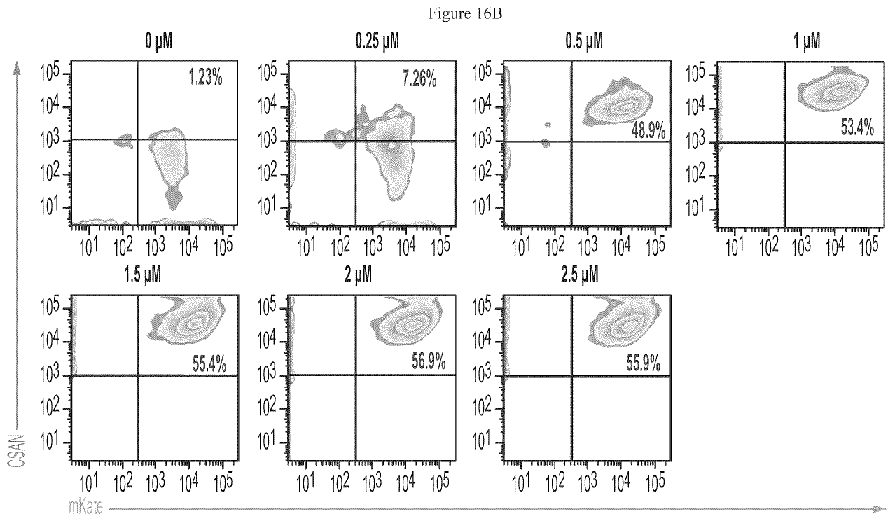

Previously, targeted farnesylated CSANs were shown to mediate reversible cell-cell interactions in a concentration-dependent manner¹⁹. To characterize the dependence of the targeted f-CSANs concentration on the stability of induced cell-cell interactions, Raji cells were modified with variable concentrations (0-2.5 μM) of αEGFR-Fn3-Far CSANs, followed by co-culture with A431-R cells and analysis with flow cytometry. The αEGFR-Fn3-Far CSANs did not induce significant cell-cell interactions at low CSAN concentrations (<1 μM). Nevertheless, maximal αEGFR-Fn3-Far CSANs cargo transfer was observed at similar minimal CSAN concentrations (<1 μM) (FIG. 16). This demonstrates that at the low CSAN concentrations for cell surface modification, the surface-inserted CSANs can efficiently transfer to the target cells upon natural cell-cell interactions without inducing additional artificial cell-cell interactions.

The Kinetics of f-CSAN Transfer is Modulated by Receptor Number and Receptor Internalization Rate.

Cellular receptors undergo differential rates of internalization and membrane expression. Consequently, given that αEGFR-Fn3-Far CSANs on sender cells undergoes receiver cell cargo transfer by initial binding to EGFR followed by internalization, the amount of cell-cell cargo transfer of targeted f-CSANs with time should be dependent on the rate of receptor internalization and the level of membrane expression. The cellular receptors EGFR, HER2 and EpCAM have been shown to have significantly different rates of internalization upon ligand binding (EGFR >HER2>EpCAM)³⁸⁻⁴⁰. In addition, the expression levels of these receptors can vary drastically among cell types. Therefore, the rate of αEGFR-Fn3-Far CSANs cargo transfer and dependence of receptor cellular expression levels was compared to αHER2-afb-Far CSANs and αEpCAM-Fn3-Far CSANs. In each case targeting ligands with similar affinities for their target receptors were chosen^{19,34}. Both αHER2-afb-Far CSANs and αEpCAM-Fn3-Far CSANs were prepared, labelled with fluorescein, and used to modify the surfaces of Raji cells. Similar numbers of Raji cells modified with similar amounts of targeted f-CSANs were co-cultured with appropriate receiver cells expressing either different levels of EGFR expression (A431-R cells, high EGFR expression; and MDA-MB-231-R cells, low EGFR expression) or similar levels of either HER2 (SK-BR-3-R) or EpCAM (HT29-R cells) (Table 1). Consistent with the 25-fold difference in EGFR expression, the time necessary for 50% of the A431-R cells receiver cells to undergo labelling with αEGFR-Fn3-Far CSANs (T_{50%}) from the modified Raji cells was found to be 23-fold faster than for MDA-MB-231-R cells (FIGS. 4A-4C, 4I and Table 1). Nevertheless, when normalized to EGFR expression (Table 1), no significant difference in the relative internalization efficiency (RIE) of the αEGFR-Fn3-Far CSANs was observed, thus, indicating that the amount of f-CSAN cargo transfer is dependent on the level of EGFR expression and that the rate of EGFR internalization is similar for the two cell lines.

When cargo transfer of αEGFR-Fn3-Far CSANs was compared to αHER2-afb-Far CSANs, αEGFR-Fn3-Far CSANs labelling (T_{50%}) of A431-R (EGFR⁺) cells was found to be 38-fold greater than αHER2-afb-Far CSANs cargo transfer to SK-BR-3-R (HER2⁺) cells, despite a 4-fold lower expression of HER2 than EGFR (FIG. 4D, 4I and Table 1). Consistent with the approximately 8-fold difference in the rate of HER2 internalization relative to the rate of EGFR internalization^{39,40}, the RIE value for αHER2-afb-Far CSANs and SK-BR-3-R (HER2⁺) cells was found to be nearly 10-fold lower than observed for αEGFR-Fn3-Far CSANs transferred to A431-R (EGFR⁺) cells. Similarly, the RIE value for αEpCAM-Fn3-Far CSANs was found to be approximately 24-fold and 2.5-fold lower than αEGFR-Fn3-Far CSANs labelling (T_{50%}) of A431-R (EGFR⁺) cells and αHER2-afb-Far CSANs for SK-BR-3-R (HER2⁺) cells, respectively (FIG. 4E,4I and Table 1). Thus, when normalized to the level of receptor expression, despite the difference in cell type, the cargo transferability of the targeted f-CSANs correlates with the rate of receptor internalization.

Modular Design of f-CSANs Facilitates Use of Single Chain Antibody Targeting Ligands.

In general, the use of the C-terminal CVIA bio-conjugation tag requires that a targeting ligand fused to the N-terminus lack disulfides to avoid the potential need to carry out refolding during purification. In particular, the incorporation of the widely used antibody single chain variable fragment (scFv) framework in the presence of additional cysteines in the fusion protein can lead to difficulties due to aggregation, even after undergoing careful refolding or expression in disulfide isomerase expressing bacterial strains^{41,42}. It has been demonstrated that farnesylated and geranylgeranylated CSANs displaying variable amounts of prenylation could be prepared by self-assembly with a mixture of prenylated monomers and unprenylated targeting monomers¹⁹ Indeed, the stability of f-CSANs composed of an average of four farnesylated DHFR² monomers was sufficient for stable binding (>48 h) to cell surfaces¹⁹.

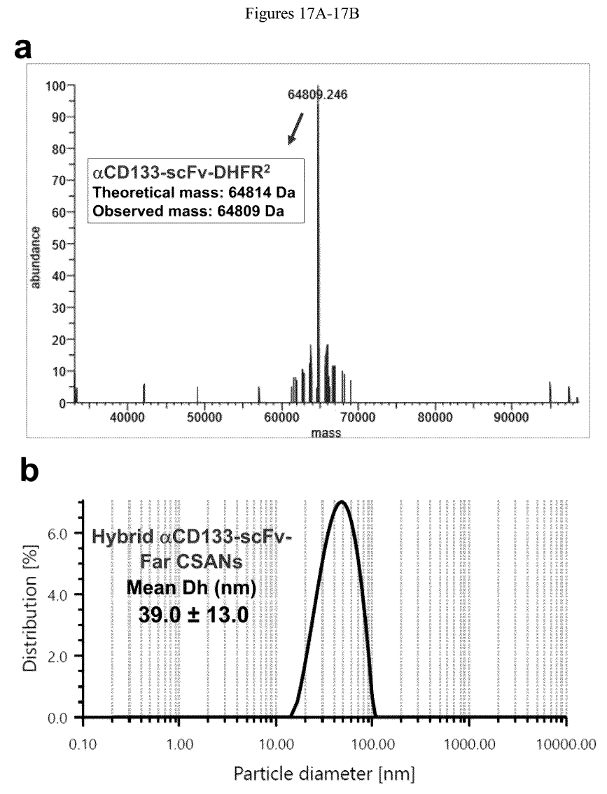

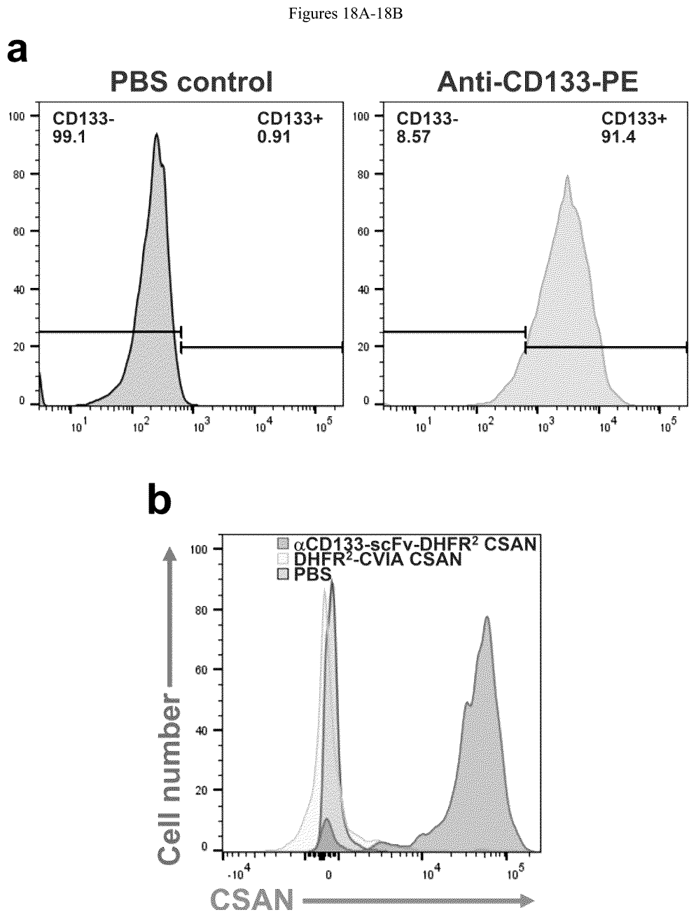

CD133 is a transmembrane protein that has been found to be associated with neural and haematopoietic stem cells, as well as cancer stem-like cells (CSC)^{43,44}. Although, stem cells and CSCs have been shown to be resistant to chemotherapeutics, potent immunotoxins targeting CD133 are able to be internalized and kill tumor cells expressing CD133⁴⁵⁻⁴⁷. Consequently, to investigate the potential for utilizing f-CSANs incorporating disulfide-containing targeting ligands for cell-cell cargo transfer, hybrid αCD133-scFv-far CSANs were self-assembled by mixing a 1:1 ratio of αCD133-scFv-DHFR² and DHFR²-Far monomers in the presence of bisMTX (FIGS. 4f and 17). Raji cells were modified with the hybrid αCD133-scFv-far CSANs (FIG. 4g), incubated with CD133′ HT29-R cells and the transfer of the fluorescein-labelled CSANs monitored over time by flow cytometry (FIG. 4h,i and 18). The T_{50%} values for αEGFR-Fn3-Far CSANs labelling of A431-R (EGFR⁺) cells was found to be 78-fold greater than hybrid αCD133-scFv-Far CSANs cargo transfer to HT29-R (CD133⁺) cells. Unfortunately, an αCD133 monoclonal antibody that binds to both glycosylated and non-glycosylated CD133 is not available, precluding our ability to accurately measure the amount of CD133 on HT29-R cells and thus directly compare the internalization behavior of CD133 with other receptors. Nevertheless, the C4T results with hybrid αCD133-scFv-Far CSANs are consistent with prior studies demonstrating that the αCD133-scFv could be employed for drug delivery⁴⁵⁻⁴⁸. Thus, hybrid targeted f-CSANs and scFvs can be used to carryout C4T.

### Dependence of Cell-Cell Cargo Transfer on Sender and Receiver Cell Type.

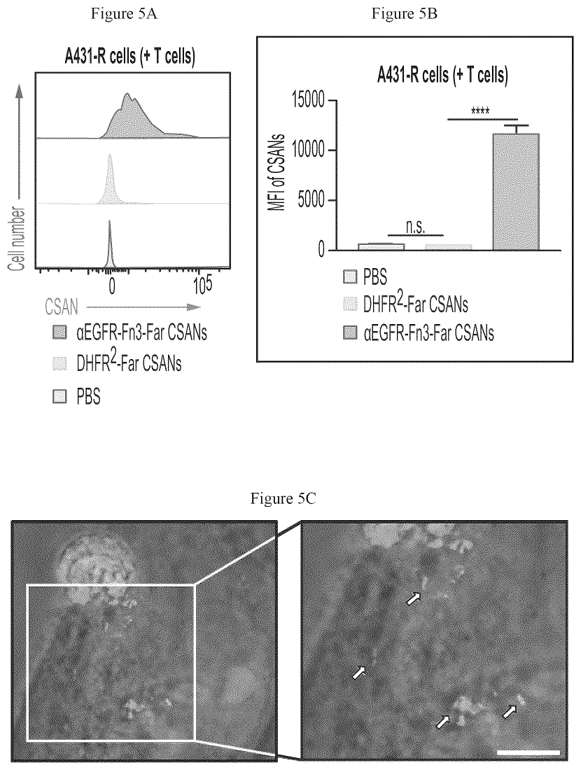

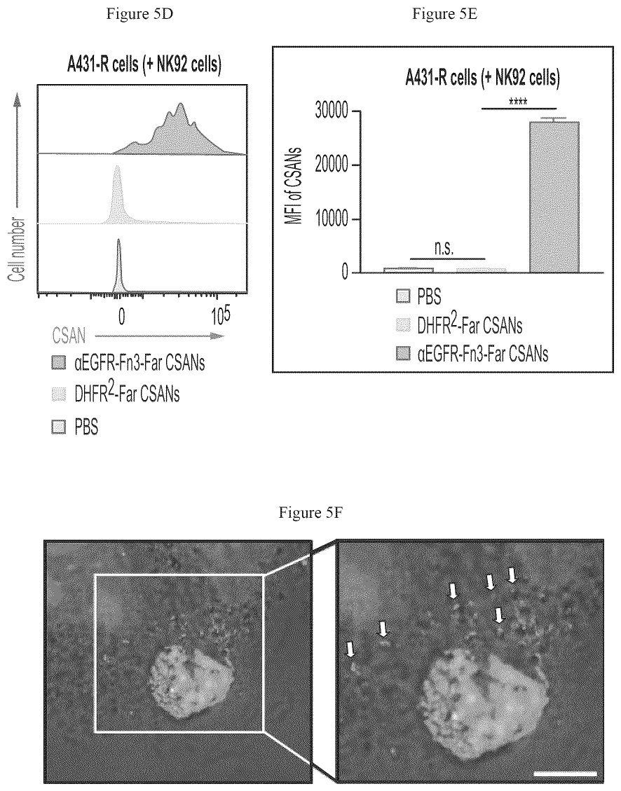

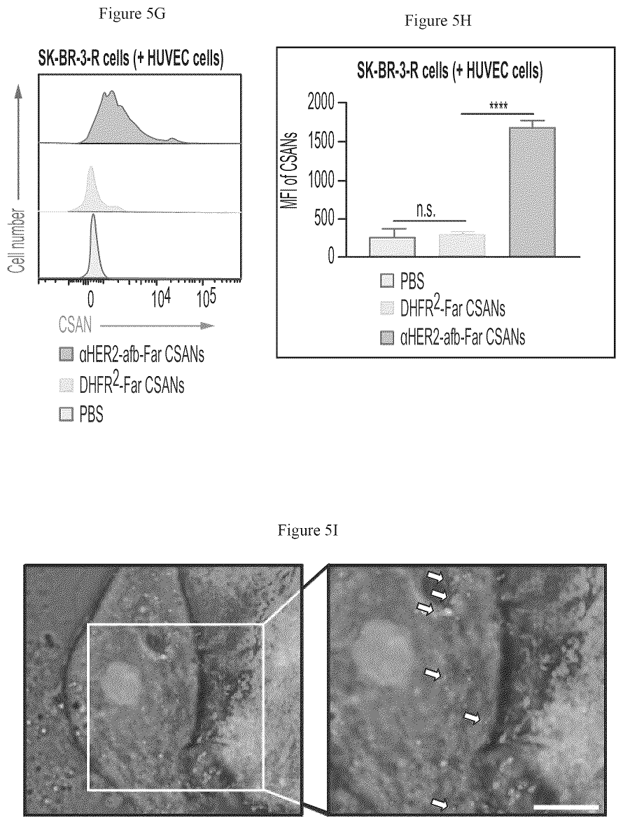

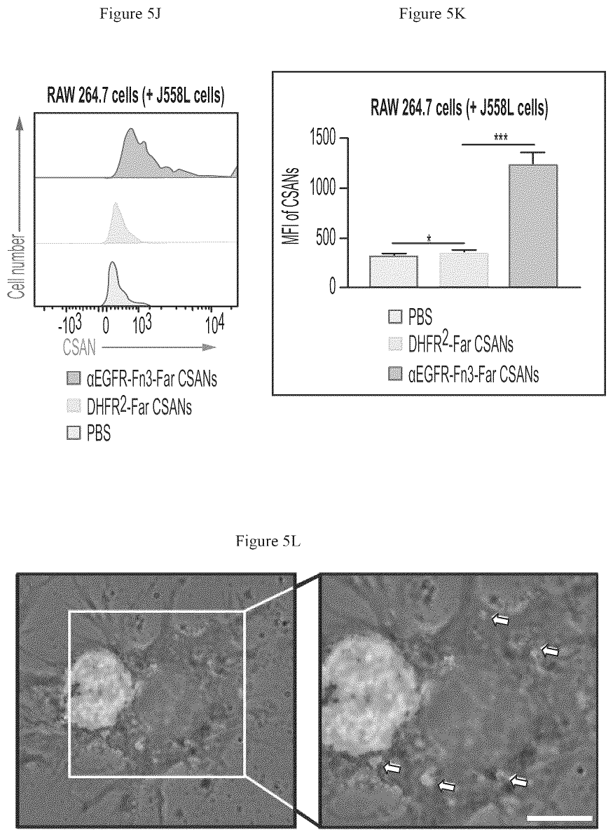

Having investigated the ability of Raji cells to serve as sender cells, the ability of different sender cells and receiver cells to carry out cargo transfer was then investigated. Cytotoxic T-lymphocytes (CD8⁺ T cells) and Natural Killer (NK) cells were modified with fluorescein-labeled αEGFR-Fn3-far CSANs or control fluorescein-labeled DHFR²-Far CSANs and their ability to facilitate cargo transfer to A431-R (EGFR⁺) cells over 30 min was determined by flow cytometry and fluorescent microscopy (FIG. 5a-f). While no significant transfer of non-targeted f-CSANs to A431-R was observed for either CD8⁺ T cells or NK cells, significant cargo transfer was observed for αEGFR-Fn3-far CSANs to the target cells. Interestingly, although the sizes of CD8⁺ T cells or NK cells are very similar, over twice as much αEGFR-Fn3-far CSANs was found to be transferred by NK cells relative to CD8⁺ T cells. Consistent with these findings, internalized fluorescently labelled punctate spots were observed in the receiver A431-R cells by fluorescent microscopy. The punctate spots, and thus labelled αEGFR-Fn3-far CSANs, could be observed throughout the cell and even near or associated within the nucleus.

TABLE 1
Transfer kinetics of different CSANs for different receiver cell lines.

| Receiver Cell | A431-R | MDA-MB-231-R | SK-BR-3-R | HT29-R (Ep- | HT29-R |
|---|---|---|---|---|---|
| Type | (EGFR+) | (EGFR+) | (HER2+) | CAM+) | (CD133+) |
| Targeted | αEGFR-Fn3-Far | αEGFR-Fn3-Far | αHER2-afb-Far | αEpCAM-Fn3-Far | hybrid |
| Farnesyl CSANs | CSAN | CSAN | CSAN | CSAN | αCD133-scFv- |
| | | | | | far CSAN |
| Receptor No. | 17,212,212 ± | 677,707 ± | 4,328,469 ± | 3,624,282 ± | |
| | 895,286 | 15,973 | 2,116,328 | 16,899 | |
| Normalized Re- | 1.0 | 0.0394 | 0.251 | 0.211 | |
| ceptor #^{a} (NR#) | | | | | |
| T_{50%}^{b} (min) | 7 | 162 | 264 | 802 | 546 |
| Relative Inter- | 1.0 | 1.1 | 0.106 | 0.041 | |
| nalization Effi- | | | | | |
| ciency^{c} (RIE) | | | | | |

^{a}Normalized Receptor # (NR#) was calculated by the ratio of Cell Receptor #/A431-R EGFR #.
^{b}T_{50%} represents the time required for 50% of the receiver cells to undergo labelling with targeted f-CSANs.
^{c}Relative Internalization Efficiency was calculated by the ratio of T_{50%}^{A431-R}/(T_{50%}^{X} · NR #).

To examine the feasibility of f-CSAN-based cargo transfer with non-lymphocytic cells, human vascular endothelial cells (HUVEC) were modified with fluorescein-labelled αHER2-afb-far CSANs or non-binding control f-CSANs and the amount of cargo transfer to SK-BR-3-R (HER2V) cells determined by flow cytometry and fluorescence microscopy. HUVEC cells were indeed shown to serve as sender cells for αHER2-afb-far CSANs transferring to SK-BR-3-R cells, with multiple fluorescein-labelled puncta observable throughout the cell (FIG. 5g-i). The potential for phagocytotic cells, such as macrophages, to serve as receiver cells was also examined. Since the αEGFR-Fn3 CSANs showed cross-reactivity with mouse EGFR, mouse myeloma cells (J558L) were modified with fluorescein-labelled αEGFR-Fn3-far CSANs and incubated with mouse macrophage RAW 264.7 cells (EGFR⁺) for 30 min. Macrophages have been shown to express EGFR, which has been shown to play a critical role in their response to pathogens⁴⁹. Significant amounts of cargo transfer of the labelled f-CSANs were observed by flow cytometry with green fluorescent puncta observable by fluorescent microscopy throughout the RAW 264.7 receiver cells including the nucleus (FIG. 5j-1). Previously, EGFR-targeted CSANs were shown to be endocytosed by macrophage RAW 264.7 cells⁵⁰. In addition, non-targeted CSANs were shown to undergo uptake by the common scavenger receptor-1 (SR-1)⁵¹. In contrast, non-specific cargo-transfer was not observed by the non-targeted f-CSANS to RAW 264.7 cells, thus SR-1 does not appear to directly induce CSANs cell-cell transfer to macrophages. Taken together, these examples indicate that f-CSANs can be used to label a variety of sender cell types in which a variety of receiver cells expressing the target receptors, for example EGFR and HER2, can carry out cargo transfer by endocytosis.

### Interaction-Dependent Drug Delivery.

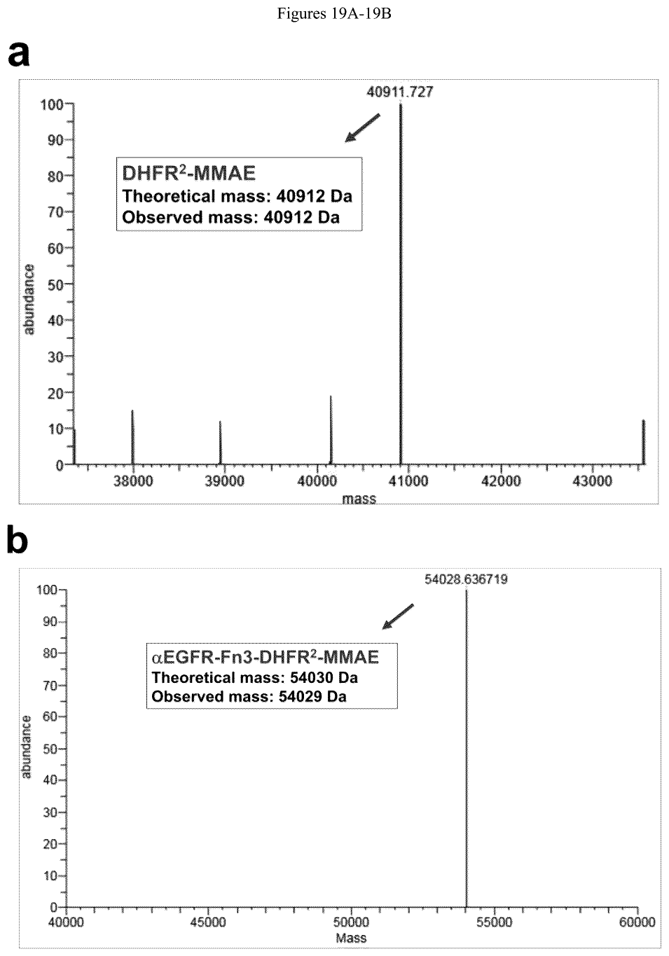

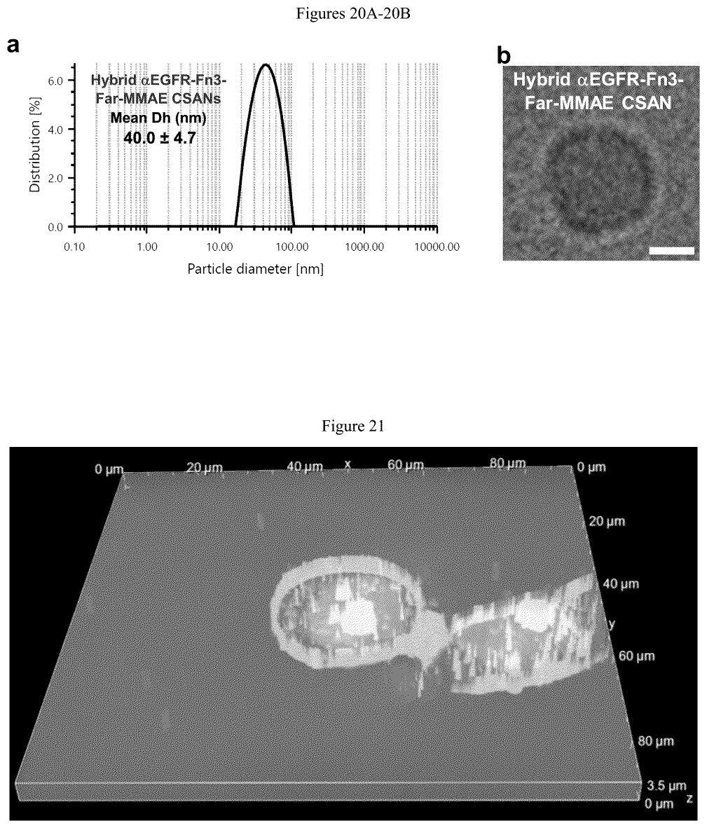

Having demonstrated that targeted f-CSANs are able to carry out cargo transfer, their potential to selectively deliver biologically active agents was investigated. Since farnesyltransferase is a promiscuous enzyme that can employ isoprenoids other than native farnesyl diphosphate as substrates⁵²⁻⁵⁴. The αEGFR-Fn3-DHFR²-CVIA monomer was prenylated with geranyl-azide diphosphate (C10-N₃-OPP) to prepare αEGFR-Fn3-DHFR²—N₃, followed by click conjugation to dibenzocyclooctyne covalently attached to valine-citrulline-p-aminobenzoyloxycarbonyl-monomethyl auristatin E conjugated through a PEG linker (DBCO-PEG₄-VC-PAB-MMAE) (FIG. 19). VC-PAB-MMAE incorporates a cathepsin sensitive valine-citrulline-PAB self-immolative linker and the potent anti-mitotic agent, MMAE. The drug linker combination has been extensively employed for the development of antibody drug conjugates, including several FDA-approved ADCs⁵⁵. The resulting protein-drug conjugate, αEGFR-Fn3-DHFR²-MMAE, was self-assembled in the presence of αEGFR-Fn3-DHFR²-Far at a 1:3 ratio, respectively, into hybrid αEGFR-Fn3-Far-MMAE CSANs (FIGS. 6a and 20). Sender Raji cells were modified with fluorescently labelled αEGFR-Fn3-Far-MMAE CSANs, followed by co-culture with A431-R cells. Within 30 mins, cargo-transfer of the hybrid αEGFR-Fn3-Far-MMAE CSANs could be observed by fluorescent microscopy with fluorescent puncta observable throughout the cell, including the nucleus (FIG. 6b, c and 21).

To assess the potential for the hybrid αEGFR-Fn3-Far-MMAE CSANs to act as drug delivery vehicles, sender Raji cells were modified with hybrid αEGFR-Fn3-Far-MMAE CSANs, followed by co-culturing with A431-R (17×10⁶ EGFR per cell) cells for 2 h. The CSAN-modified Raji cells were then removed from the co-culture and the A431-R cells cultured for 96 h, followed by cell viability quantification. Co-culturing with Raji cells modified with the αEGFR-Fn3-Far-MMAE CSANs resulted in potent cytotoxicity against A431-R cells, while the unmodified Raji cells or Raji cells modified with non-targeting hybrid DHFR²-Far-MMAE CSANs failed to exert any significant cytotoxicity toward the receiver cells (FIG. 6d). To assess the role of EGFR expression on the cargo transfer induced cytotoxicity by the hybrid αEGFR-Fn3-Far-MMAE CSANs, a similar cytotoxicity study was carried out with MDA-MB-231-R cells and MDA-MB-453-R, which express 25-fold and 8500-fold less EGFR than A431-R cells, respectively. Clearly, the expression levels of EGFR on the receiver cells impacted the ability of the sender Raji cells modified with hybrid αEGFR-Fn3-Far-MMAE CSANs to carry out cargo transfer induced cytotoxicity, since a 6-fold reduction in cytotoxicity was observed for MDA-MB-231-R receiver cells, while no significant cytotoxicity was observed for the MDA-MB-453-R cells (FIG. 6e, f).

### Interaction-Dependent Delivery of Oligonucleotides.

Given that targeted f-CSANs are able to facilitate cargo transfer of small molecules, their ability to carry out macromolecular delivery by assessing oligonucleotide transfer was investigated. αEGFR-Fn3-DHFR²—N₃ was treated with a deoxy-oligonucleotide modified with a DBCO group at the 5′-terminus and AlexaFluor-488 fluorophore at the 3′-terminus (FIG. 22). The resulting protein-oligonucleotide-conjugate, αEGFR-Fn3-DHFR²-ssDNA-AF488, was self-assembled in the presence of αEGFR-Fn3-DHFR²-Far at a 1:3 ratio, respectively, into hybrid αEGFR-Fn3-Far-ssDNA-AF488 CSANs (FIGS. 7a and 23). The plasma membranes of the sender Raji cells were modified with the hybrid CSANs, and 5-fold more of the fluorescently labeled hybrid αEGFR-Fn3-Far-ssDNA-AF488 CSANs was observed to have been transferred to the receiver A431-R cells at 37° C. compared with 4° C. (FIG. 7b).

Previously, both bivalent and octavalent anti-αᵥβ₃ CSANs composed of DHFR²-cyclic-RGD monomers and bisMTX conjugated to the anti-eIF4E antisense oligonucleotides (ASO), KDssDNA, were shown to knock-down eIF4E translation in MDA-MB-231 cells by approximately 50%, when compared to anti-αᵥβ₃ CSANs composed of a scrabbled DNA cargo⁵⁶. Consequently, given that EGFR targeting has been used for nanoparticle-based delivery of a variety of nucleic acids⁵⁷, the hybrid αEGFR-Fn3-Far-KDssDNA CSANs were prepared by oligomerizing αEGFR-Fn3-DHFR²-KDssDNA and αEGFR-Fn3-DHFR²-Far at a 1:3 ratio. Raji sender cells were modified with either the hybrid αEGFR-Fn3-Far-KDssDNA CSANs or the hybrid αEGFR-Fn3-Far-CTRLssDNA CSANs, which contained a scrambled control ssDNA (CTRLssDNA). After co-culturing of the modified sender Raji cells with MDA-MB-231 cells for 48 h, the amount of intracellular eIF4E was quantified by western blot analysis. Compared to Raji cells modified with αEGFR-Fn3-Far-CTRLssDNA CSANs, a greater than 40% reduction in the amount of eIF4E was observed for MDA-MB-231 receiver cells co-cultured with Raji sender cells modified with the hybrid αEGFR-Fn3-Far-KDssDNA CSANs (FIG. 7c, d). Although not optimized for either f-CSAN concentration, the sender cell/receiver cell ratio, conjugation linker length, or intracellular ASO release, the ability to observe significant activity for the anti-eIF4E ASO suggests that cell-cell cargo transfer can be used for functional cell-based delivery of not just small molecules, but also macromolecules.

### Discussion

Cell-cell communication is carried out by either a soluble hormone with an extracellular or intercellular receptor or the direct engagement of a membrane bound ligand with a membrane bound receptor. Of these three modes of contact, the direct contact between two different cells can result not just in receptor activation and intracellular signal transduction, but in many cases the direct transfer and internalization of the ligand from one cell membrane to the other cell⁵⁸⁻⁶³. The amount of transfer from sender cells can span from a few ligands to significant amounts of the sender cell membrane, referred to as trogocytosis. Trogocytosis has been observed between immune cells and antigen presenting cells, neurons and microglia, parasites, endothelia cells and CAR-T-cells and target cells, to name a few^{58,61,62,64}. Recently, Tang and co-workers have pioneered the development of a cargo transfer-based approach for monitoring cell-cell interactions¹⁶. Sender cells are genetically engineered to express GFP or a GFP analog fused to a membrane displaying domain. Cargo transfer is then facilitated by receiver cells expressing an anti-GFP nanobody fused to an internalizing membrane domain, referred to as GFP-based Touching Nexus, or G-baToN. Interestingly, the G-baToN approach enables cell-based delivery of fluorophores, proteins and nucleic acids from sender to receiver cells as possible tools for monitoring cell-cell interactions¹⁶. In this Example, a non-genetic biomimetic trogocytosis approach was developed for engineering cell-cell cargo transfer that could be employed with a variety of cell types, irrespective of their ability to be stably transfected, and potentially useful for targeted cell-based drug delivery, as well as cell-cell interaction monitoring.

Our laboratory has demonstrated that targeted chemically self-assembled nanorings (CSANs) can be prepared by incubation of DHFR-DHFR (DHFR²) fusion proteins recombinantly coupled to a targeting single chain antibody (scFv), fibronectin (Fn3), affibody (afb) or peptide^{22,34,50,56}. CSANs that can covalently or non-covalently couple modified non-natural phospholipids have been shown to modify cell membranes^{19,33,65}. Recently, it has been demonstrated that DHFR² monomers fused with a C-terminal CVIA sequence can be farnesylated or geranylgeranylated by farnesyltransferase or geranylgeranyltransferase respectively. Self-assembly of the prenylated monomers targeted to specific cell surface antigens, resulted in prenylated CSANs capable of inducing specific cell-cell interactions, such as T-cell killing of the target tumor cells¹⁹. Nevertheless, although the prenylated CSANs were found to stably bind to cell membranes for days, due to their multivalency, in this Example whether receptor targeted farnesyl CSANs on sender cells could undergo cargo transfer through energy dependent internalization of the targeted plasma membrane receptor was studied.

Analysis of the temperature dependence of targeted f-CSANs cargo transfer demonstrated that cargo transfer from sender cells to receiver cells was carried out by energy dependent receptor internalization (FIG. 15). In addition, when compared across receptors and considering that the binding affinities of the monomeric binding ligands are similar (<10-fold), the extent of cargo transfer was found to be dependent on the interplay between receptor internalization rate and level of receptor expression. For example, when comparing EGFR based cargo transfer to cell lines (A431-R vs MDA-MB-231-R) differing in EGFR expression by 25-fold, the amount of cargo transfer was found to be similarly decreased by 23-fold for the lower expressing cell line (MDA-MB-231-R) (Table 1). Nevertheless, as expected, the relative internalization efficiency (RIE) for the targeted receptor was found to be similar, indicating that the rate of EGFR internalization is independent of the cell type (Table 1). Similarly, when the amount of cargo transfer between two cell lines expressing similar levels of receptors, but with different internalization rates, was compared, cells expressing the faster internalizing receptor (HER2, SK-BR-3-R) accumulated a greater amount of f-CSANs than cells expressing the slower internalizing receptor (EpCAM, HT29-R). Using EGFR expression by A431-R cells as a benchmark, the RIE could be determined for EGFR, HER2 and EpCAM, with EGFR being the most efficient and EpCAM the least efficient (Table 1). Consistent with these findings, the rate of EGFR internalization has been found to be 8- to 10-fold faster than HER2 and >10-fold faster than EpCAM, depending on the cellular differentiation conditions³⁸⁻⁴⁰ (Table 1). Consequently, targeted farnesyl CSANs cargo transfer can be used to assess the RIE of cell surface receptors for targeting ligands, if the number of receptors is known and the time necessary to transfer the targeted f-CSANs to 50% of the cells is known (Table 1).

Given the modularity of CSANs, hybrid αCD133-scFv-Far CSANs were prepared from DHFR²-Far and αCD133-scFv-DHFR². The hybrid αCD133-scFv-Far CSANs were shown to bind stably to the sender cell membranes and to carry out cargo transfer to CD133⁺ HT29-R cells. Interestingly, αEpCAM-Fn3-Far CSANs and the hybrid αCD133-scFv-Far CSANs were shown to carry out similar levels of cargo transfer over a similar time frame. Thus, both f-CSANs could be used to carry out cargo transfer to the same cells over the same time enabling the development of multi-specific receptor-based cargo transfer.

The modular nature of CSANs self-assembly suggested that delivery of conjugated payloads might also be assessable. Hybrid αEGFR-Fn3-Far-MMAE CSANs were prepared by bisMTX facilitated self-assembly of αEGFR-Fn3-DHFR²-MMAE and αEGFR-Fn3-DHFR²-Far. Sender cells loaded with the hybrid αEGFR-Fn3-Far-MMAE CSANs delivered the drug-loaded f-CSANs followed by the induction of cell death. Consistent with the finding that the efficiency of cargo transfer is dependent on receptor expression, the amount of observed cytotoxicity was found to be dependent on EGFR expression levels. Similarly, hybrid αEGFR-Fn3-Far-ssDNA CSANs were prepared from αEGFR-Fn3-DHFR²-ssDNA and αEGFR-Fn3-DHFR²-Far and found to efficiently transfer oligonucleotides from sender cells to receiver cells. In addition, if the oligonucleotide was an anti-sense oligonucleotide targeting the translation initiation factor eIF4E, significant knockdown of the amount of eIF4E was observed in the receiver cells. Importantly, the sender cells were not affected by the targeted f-CSANs cargos bound to their membranes. Thus, targeted f-CSANs could potentially be used for drug delivery to tissues that the sender cells have an affinity to. For example, the natural affinity of mesenchymal stem cells for tumor sites has been shown to enhance tumor targeting and penetration without adverse off-target effects and tumor penetration challenges typically observed for nano and micro-based drug delivery carriers⁶⁶. In addition, in principle, multiple drugs or oligonucleotides could be loaded onto sender cells by simply incubating mixtures of targeted f-CSANs conjugated to specific payloads with sender cells. Importantly, unlike other nanoparticle delivery approaches, the tissue localization and biodistribution of the CSANs will likely be dependent on the tissue penetration and biodistribution of the cells and not the nanorings. In essence, regardless of what is being delivered, targeted f-CSANs based cell-cell cargo transfer can be used to non-genetically engineer synthetic cell-cell interaction tracking and communications. Chemical messages can be delivered by sender cells to specific receiver cells in order to mark them or induce a desired response, be it apoptosis or alternations in signal transduction pathways. With the inherent modular nature and universal membrane binding ability of the C4T approach, multi-targeted cargo transfer can be explored from any sender cell of choice with potentially greater cargo transfer specificity obtained to the receiver cell of choice. Thus, C4T is a biomimetic trogocytosis approach with the potential to be a broadly applicable macro-chemical biological tool for the assessment of cellular interactions and engineering of cell-to-cell communications.

In one embodiment Wang Y., et al. (2022) Engineering Biomimetic Trogocytosis with Farnesylated Chemically Self-Assembled Nanorings. Biomacromolecules 23(12) 5018-5035 is incorporated by reference herein.

In one embodiment Wang Y., et al. (2022) Macro-Chemical Biology: Engineering Biomimetic Trogocytosis with Farnesylated Chemically Self-Assembled Nanorings, doi.org/10.1101/2022.03.01.482559 is incorporated by reference herein.

In one embodiment Wang Y., et al. (2021) Engineering reversible cell-cell interactions using enzymatically lipidated chemically self-assembled nanorings, Chem Sci. 12(1):331-340 is incorporated by reference herein.

### DOCUMENTS IN EXAMPLE 1

(1) Friedl, P., and Bröcker, E. B. (2002) TCR triggering on the move: Diversity of T-cell interactions with antigen-presenting cells. Immunol. Rev. 186, 83-89.
(2) Cheung, K. J., and Ewald, A. J. (2014) Illuminating breast cancer invasion: Diverse roles for cell-cell interactions. Curr. Opin. CellBiol 30, 99-111.
(3) Kamińska, K., Szczylik, C., Bielecka, Z. F., Bartnik, E., Porta, C., Lian, F., and Czarnecka, A. M. (2015) The role of the cell-cell interactions in cancer progression. J. Cell. Mol. Med. 19, 283-296.
(4) Parekkadan, B., Berdichevsky, Y., Irimia, D., Leeder, A., Yarmush, G., Toner, M., Levine, J. B., and Yarmush, M. L. (2008) Cell-cell interaction modulates neuroectodermal specification of embryonic stem cells. Neurosci. Lett. 438, 190-195.
(5) Akins, M. R., and Biederer, T. (2006) Cell-cell interactions in synaptogenesis. Curr. Opin. Neurobiol. 16, 83-89.
(6) Levine, J. F., and Stockdale, F. E. (1985) Cell-cell interactions promote mammary epithelial cell differentiation. J. Cell Biol. 100, 1415-1422.
(7) Rakic, P. (1981) Neuronal-glial interaction during brain development. Trends Neurosci. 4, 184-187.
(8) Banerjee, S., and Bhat, M. A. (2007) Neuron-glial interactions in blood-brain barrier formation. Annu. Rev. Neurosci. 30, 235-258.
(9) Bedoui, S., and Gebhardt, T. (2011) Interaction between dendritic cells and T cells during peripheral virus infections: A role for antigen presentation beyond lymphoid organs?Curr. Opin. Immunol. 23, 124-130.
(10) Woodland, D. L., and Kohlmeier, J. E. (2009) Migration, maintenance and recall of memory T cells in peripheral tissues. Nat. Rev. Immunol. 9, 153-161.
(11) Waldman, A. D., Fritz, J. M., and Lenardo, M. J. (2020) A guide to cancer immunotherapy: from T cell basic science to clinical practice. Nat. Rev. Immunol. 20, 651-668.
(12) Mao, A. S., and Mooney, D. J. (2015) Regenerative medicine: Current therapies and future directions. Proc. Natl. Acad. Sci. U.S.A. 112, 14452-14459.
(13) Gomes, M. E., Rodrigues, M. T., Domingues, R. M. A., and Reis, R. L. (2017) Tissue engineering and regenerative medicine: new trends and directions—A year in review. Tissue Eng. —Part B Rev. 23, 211-224.
(14) Porterfield, W. B., and Prescher, J. A. (2015) Tools for visualizing cell-cell “interactomes.” Curr. Opin. Chem. Biol 24, 121-130.
(15) Bechtel, T. J., Reyes-Robles, T., Fadeyi, O. O., and Oslund, R. C. (2021) Strategies for monitoring cell-cell interactions. Nat. Chem. Biol 17, 641-652.
(16) Tang, R., Murray, C. W., Linde, I. L., Kramer, N. J., Lyu, Z., Tsai, M. K., Chen, L. C., Cai, H., Gitler, A. D., Engleman, E., Lee, W., and Winslow, M. M. (2020) A versatile system to record cell-cell interactions. Elife 9, 1.
(17) Pasqual, G., Chudnovskiy, A., Tas, J. M. J., Agudelo, M., Schweitzer, L. D., Cui, A., Hacohen, N., and Victora, G. D. (2018) Monitoring T cell-dendritic cell interactions in vivo by intercellular enzymatic labelling. Nature 553, 496-500.
(18) Csizmar, C. M., Petersburg, J. R., and Wagner, C. R. (2018) Programming Cell-Cell Interactions through Non-genetic Membrane Engineering. Cell Chem. Biol. 25, 931-940.
(19) Wang, Y., Kilic, O., Csizmar, C. M., Ashok, S., Hougland, J. L., Distefano, M. D., and Wagner, C. R. (2021) Engineering reversible cell-cell interactions using enzymatically lipidated chemically self-assembled nanorings. Chem. Sci. 12, 331-340.
(20) Carlson, J. C. T., Jena, S. S., Flenniken, M., Chou, T. F., Siegel, R. A., and Wagner, C. R. (2006) Chemically controlled self-assembly of protein nanorings. J. Am. Chem. Soc. 128, 7630-7638.
(21) Li, Q., So, C. R., Fegan, A., Cody, V., Sarikaya, M., Vallera, D. A., and Wagner, C. R. (2010) Chemically self-assembled antibody nanorings (CSANs): Design and characterization of an anti-CD3 IgM biomimetic. J. Am. Chem. Soc. 132, 17247-17257.
(22) Shen, J., Vallera, D. A., and Wagner, C. R. (2015) Prosthetic antigen receptors. J. Am. Chem. Soc. 137, 10108-10111.
(23) Hougland, J. L., Hicks, K. A., Hartman, H. L., Kelly, R. A., Watt, T. J., and Fierke, C. A. (2010) Identification of Novel Peptide Substrates for Protein Farnesyltransferase Reveals Two Substrate Classes with Distinct Sequence Selectivities. J. Mol. Biol. 395, 176-190.
(24) Turek-Etienne, T. C., Strickland, C. L., and Distefano, M. D. (2003) Biochemical and structural studies with prenyl diphosphate analogues provide insights into isoprenoid recognition by protein farnesyl transferase. Biochemistry 42, 3716-3724.
(25) Lane, K. T., and Beese, L. S. (2006) Structural biology of protein farnesyltransferase and geranylgeranyltransferase type I. J. Lipid Res. 47, 681-699.
(26) Zhang, Y., Park, K. Y., Suazo, K. F., and Distefano, M. D. (2018) Recent progress in enzymatic protein labelling techniques and their applications. Chem. Soc. Rev. 47, 9106-9136.
(27) Duckworth, B. P., Xu, J., Taton, T. A., Guo, A., and Distefano, M. D. (2006) Site-specific, covalent attachment of proteins to a solid surface. Bioconjug. Chem. 17, 967-974.
(28) Pujari, S. S., Zhang, Y., Ji, S., Distefano, M. D., and Tretyakova, N. Y. (2018) Site-specific cross-linking of proteins to DNA: Via a new bioorthogonal approach employing oxime ligation. Chem. Commun. 54, 6296-6299.
(29) Zhang, Y., Blanden, M. J., Sudheer, C., Gangopadhyay, S. A., Rashidian, M., Hougland, J. L., and Distefano, M. D. (2015) Simultaneous Site-Specific Dual Protein Labeling Using Protein Prenyltransferases. Bioconjug. Chem. 26, 2542-2553.
(30) Rashidian, M., Kumarapperuma, S. C., Gabrielse, K., Fegan, A., Wagner, C. R., and Distefano, M. D. (2013) Simultaneous dual protein labeling using a triorthogonal reagent. J. Am. Chem. Soc. 135, 16388-16396.
(31) Suazo, K. F., Park, K., and Distefano, M. D. (2021) A Not-So-Ancient Grease History: Click Chemistry and Protein Lipid Modifications. Chem. Rev. 121, 7178-7248.
(32) Stem, L. A., Csizmar, C. M., Woldring, D. R., Wagner, C. R., and Hackel, B. J. (2017) Titratable Avidity Reduction Enhances Affinity Discrimination in Mammalian Cellular Selections of Yeast-Displayed Ligands. ACS Comb. Sci. 19, 315-323.
(33) Csizmar, C. M., Petersburg, J. R., Hendricks, A., Stem, L. A., Hackel, B. J., and Wagner, C. R. (2018) Engineering Reversible Cell-Cell Interactions with Lipid Anchored Prosthetic Receptors. Bioconjug. Chem. 29, 1291-1301.
(34) Csizmar, C. M., Petersburg, J. R., Perry, T. J., Rozumalski, L., Hackel, B. J., and Wagner, C. R. (2019) Multivalent Ligand Binding to Cell Membrane Antigens: Defining the Interplay of Affinity, Valency, and Expression Density. J. Am. Chem. Soc. 141, 251-261.
(35) Kilic, O., De Souza, M. R. M., Almotlak, A. A., Wang, Y., Siegfried, J. M., Distefano, M. D., and Wagner, C. R. (2020) Anti-EGFR Fibronectin Bispecific Chemically Self-Assembling Nanorings (CSANs) Induce Potent T Cell-Mediated Antitumor Responses and Downregulation of EGFR Signaling and PD-1/PD-L1 Expression. J. Med. Chem. 63, 10091-10532.
(36) Feldwisch, J., Tolmachev, V., Lendel, C., Herne, N., Sjöberg, A., Larsson, B., Rosik, D., Lindqvist, E., Fant, G., Hoiden-Guthenberg, I., Galli, J., Jonasson, P., and Abrahmsén, L. (2010) Design of an Optimized Scaffold for Affibody Molecules. J. Mol. Biol. 398, 232-247.
(37) Swaminathan, S. K., Niu, L., Waldron, N., Kalscheuer, S., Zellmer, D. M., Olin, M. R., Ohlfest, J. R., Vallera, D. A., and Panyam, J. (2013) Identification and characterization of a novel scFv recognizing human and mouse CD133. Drug Deliv. Transl. Res. 3, 143-151.
(38) Stuber, J. C., Kast, F., and Pluckthun, A. (2019) High-Throughput Quantification of Surface Protein Internalization and Degradation. ACS Chem. Biol. 14, 1154-1163.
(39) Hendriks, B. S., Opresko, L. K., Wiley, H. S., and Lauffenburger, D. (2003) Coregulation of epidermal growth factor receptor/human epidermal growth factor receptor 2 (HER2) levels and locations: Quantitative analysis of HER2 overexpression effects. Cancer Res. 63, 1130-1137.
(40) Hendriks, B. S., Opresko, L. K., Wiley, H. S., and Lauffenburger, D. (2003) Quantitative analysis of HER2-mediated effects on HER2 and epidermal growth factor receptor endocytosis. Distribution of homo- and heterodimers depends on relative HER2 levels. J. Biol. Chem. 278, 23343-23351.
(41) Wang, W., Nema, S., and Teagarden, D. (2010) Protein aggregation-Pathways and influencing factors. Int. J. Pharm. 390, 89-99.
(42) Liu, H., and May, K. (2012) Disulfide bond structures of IgG molecules: Structural variations, chemical modifications and possible impacts to stability and biological function. MAbs 4, 17-23.
(43) Wu, Y., and Wu, P. Y. (2009) CD133 as a marker for cancer stem cells: Progresses and concerns. Stem Cells Dev. 18, 1127-1134.
(44) Jang, J. W., Song, Y., Kim, S. H., Kim, J., and Seo, H. R. (2017) Potential mechanisms of CD133 in cancer stem cells. Life Sci. 184, 25-29.
(45) Olsen, C. E., Cheung, L. H., Weyergang, A., Berg, K., Vallera, D. A., Rosenblum, M. G., and Selbo, P. K. (2020) Design, characterization, and evaluation of scFvCD133/rgelonin: A CD133-targeting recombinant immunotoxin for use in combination with photochemical internalization. J. Clin. Med. 9, 1-16.
(46) Ohlfest, J. R., Zellmer, D. M., Panyam, J., Swaminathan, S. K., Oh, S., Waldron, N. N., Toma, S., and Vallera, D. A. (2013) Immunotoxin targeting CD133+ breast carcinoma cells. Drug Deliv. Transl. Res. 3, 195-204.
(47) Skubitz, A. P. N., Taras, E. P., Boylan, K. L. M., Waldron, N. N., Oh, S., Panoskaltsis-Mortari, A., and Vallera, D. A. (2013) Targeting CD133 in an in vivo ovarian cancer model reduces ovarian cancer progression. Gynecol. Oncol. 130, 579-587.
(48) Glumac, P. M., and LeBeau, A. M. (2018) The role of CD133 in cancer: a concise review. Clin. Transl. Med. 7, 1-14.
(49) Hardbower, D. M., Singh, K., Asim, M., Verriere, T. G., Olivares-Villagomez, D., Barry, D. P., Allaman, M. M., Washington, M. K., Peek, R. M., Piazuelo, M. B., and Wilson, K. T. (2016) EGFR regulates macrophage activation and function in bacterial infection. J. Clin. Invest. 126, 3296-3312.
(50) Shah, R., Petersburg, J., Gangar, A. C., Fegan, A., Wagner, C. R., and Kumarapperuma, S. C. (2016) In vivo evaluation of site-specifically PEGylated chemically self-assembled protein nanostructures. Mol. Pharm. 13, 2193-2203.
(51) Shen, J. (2016) Self-Assembled Antibody Nanorings as Prosthetic Antigen Receptors for Redirecting T Cells against Tumor Cells and as Platform for Delivery of Vaccine Adjuvants for Cancer Immunotherapy. UNIVERSITY OF MINNESOTA.
(52) Rashidian, M., Kumarapperuma, S. C., Gabrielse, K., Fegan, A., Wagner, C. R., and Distefano, M. D. (2013) Simultaneous dual protein labeling using a triorthogonal reagent. J. Am. Chem. Soc. 135, 16388-16396.
(53) Duckworth, B. P., Zhang, Z., Hosokawa, A., and Distefano, M. D. (2007) Selective labeling of proteins by using protein farnesyltransferase. ChemBioChem 8, 98-105.
(54) DeGraw, A. J., Palsuledesai, C., Ochocki, J. D., Dozier, J. K., Lenevich, S., Rashidian, M., and Distefano, M. D. (2010) Evaluation of alkyne-modified isoprenoids as chemical reporters of protein prenylation. Chem. Biol. Drug Des. 76, 460-471.
(55) Tong, J. T. W., Harris, P. W. R., Brimble, M. A., and Kavianinia, I. (2021) An insight into fda approved antibody-drug conjugates for cancer therapy. Molecules 26.
(56) Gangar, A., Fegan, A., Kumarapperuma, S. C., Huynh, P., Benyumov, A., and Wagner, C. R. (2013) Targeted delivery of antisense oligonucleotides by chemically self-assembled nanostructures. Mol. Pharm. 10, 3514-3518.
(57) Nguyen, P. V., Allard-Vannier, E., Chourpa, I., and Herve-Aubert, K. (2021) Nanomedicines functionalized with anti-EGFR ligands for active targeting in cancer therapy: Biological strategy, design and quality control. Int. J. Pharm. 605.
(58) Bettadapur, A., Miller, H. W., and Ralston, K. S. (2020) crossm Infection, and Disease 1-15.
(59) Qureshi, O. S., Zheng, Y., Nakamura, K., Attridge, K., Manzotti, C., Schmidt, E. M., Baker, J., Jeffery, L. E., Kaur, S., Briggs, Z., Hou, T. Z., Futter, C. E., Anderson, G., Walker, L. S. K., and Sansom, D. M. (2011) Trans-endocytosis of CD80 and CD86: A molecular basis for the cell-extrinsic function of CTLA-4. Science (80-.). 332, 600-603.
(60) Generous, A. R., Harrison, O. J., Troyanovsky, R. B., Mateo, M., Navaratnarajah, C. K., Donohue, R. C., Pfaller, C. K., Alekhina, O., Sergeeva, A. P., Indra, I., Thornburg, T., Kochetkova, I., Billadeau, D. D., Taylor, M. P., Troyanovsky, S. M., Honig, B., Shapiro, L., and Cattaneo, R. (2019) Trans-endocytosis elicited by nectins transfers cytoplasmic cargo, including infectious material, between cells. J. Cell Sci. 132.
(61) Ahmed, K. A., and Xiang, J. (2011) Mechanisms of cellular communication through intercellular protein transfer. J. Cell. Mol. Med. 15, 1458-1473.
(62) Ahmed, K. A., Munegowda, M. A., Xie, Y., and Xiang, J. (2008) Intercellular trogocytosis plays an important role in modulation of immune responses. Cell. Mol. Immunol. 5, 261-269.
(63) Davis, D. M. (2007) Intercellular transfer of cell-surface proteins is common and can affect many stages of an immune response. Nat. Rev. Immunol. 7, 238-243.
(64) Hamieh, M., Dobrin, A., Cabriolu, A., van der Stegen, S. J. C., Giavridis, T., Mansilla-Soto, J., Eyquem, J., Zhao, Z., Whitlock, B. M., Miele, M. M., Li, Z., Cunanan, K. M., Huse, M., Hendrickson, R. C., Wang, X., Rivibre, I., and Sadelain, M. (2019) CAR T cell trogocytosis and cooperative killing regulate tumour antigen escape. Nature 568, 112-116.
(65) Gabrielse, K., Gangar, A., Kumar, N., Lee, J. C., Fegan, A., Shen, J. J., Li, Q., Vallera, D., and Wagner, C. R. (2014) Reversible Re-programing of Cell-Cell Interactions. Angew. Chemie—Int. Ed. 53, 5112-5116.
(66) Krueger, T. E. G., Thorek, D. L. J., Denmeade, S. R., Isaacs, J. T., and Brennen, W. N. (2018) Concise Review: Mesenchymal Stem Cell-Based Drug Delivery: The Good, the Bad, the Ugly, and the Promise. Stem Cells Transl. Med. 7, 651-663.

### Example 2

### Shear Stress

### Experimental

1 μM DHFR²-CVIA was reduced using DTT on ice for 1 hr. 1 μM DHFR²-CVIA was then farnesylated using farnesyl diphosphate (30 μM) and farnesyltransferase (400 nM) overnight at 32° C. After 12 hrs, excess farnesyl diphosphate was removed using 10 Kda MWCO filters. The DHFR²-Farn monomers were then made into CSANs using 3 fold excess Bis-MTX for 1 hr at room temp. The CSANs were then non-specifically fluorescently labeled with NHS-Ester AlexaFluor 647 for 2 hrs at room temp in the dark. Excess fluorophore was removed by 10 Kda MWCO filters. 200,000 live GFP-Mesenchymal stem cells (MSCs) were harvested and washed with 1 mL ice cold PBS. The cells were modified with 1μM Farnesylated CSANs. The CSANs were allowed to modify the cells for 2 hrs at room temp with rotation. After 2 hrs, the cells were washed twice with 1 mL ice cold PBS. The cells modified with CSANs were then sheared under physiologically relevant conditions (7 Pa) and high stress conditions outside what is physiologically relevant (20 Pa) using a 1 mL syringe (VWR International, cat. 53548-001) and 18G needle (McKesson MedSurg, cat. 16-N18105). Flow rate was calculated based on the following equation: (Q—flow rate, Tmax—shear stress, R—inner radius of the needle, n-dynamic viscosity of solvent). Cells were analyzed via flow cytometry before shearing, sheared and then immediately re-analyzed to give real-time analysis.

$$\\ Q = \frac{π T max {R}^{{}_{} 3}}{4 n}$$

### Rationale and Key Findings

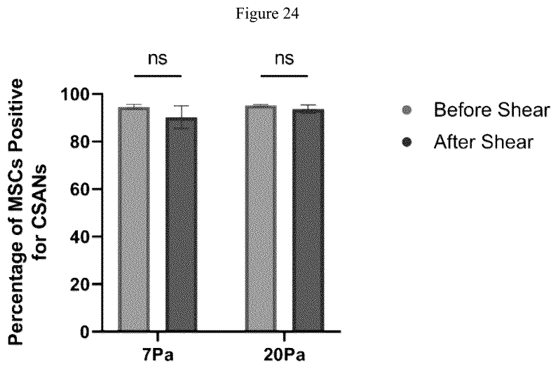

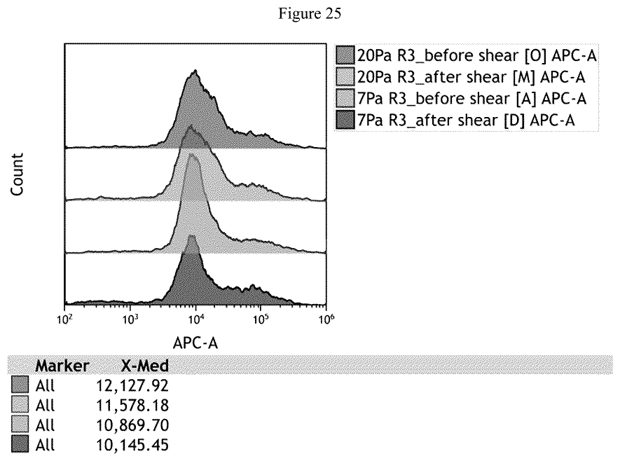

Shear stress is a physiologically relevant parameter that is exerted on cells by the blood vessel walls as cells travel throughout the body. In human arteries the shear stress present is 1-7 Pa and in the veins 0.1-0.6 Pa (Wahlberg, B., et. al. Ex vivo biomechanical characterization of syringe-needle ejections) for intracerebral cell delivery. Sci. Rep. 8, 2018, 9194). Thus, given the physiological relevance, it was investigated if farnesylated CSANs were able to stably modify the cells and remain on the cell surface under shear stress conditions that mimic the human blood vessels. Cells were subjected to two different shear stresses, with 7 Pa being the high end of what is physiologically relevant and 20 Pa mimicking conditions more akin to intravenous injection. It was found that there was no significant difference in the number of cells positive for CSANs on their cell surface (FIG. 24) nor was there a significant difference in the MFI of CSANs on the cell surface (FIG. 25). This indicates that not only does the ratio of cells positive for CSANs remain the same under shear stress conditions, but also the amount of CSANs present on those cells remains constant. These findings indicate CSANs are not lost on the surface of the cells under shear stress conditions.

### Example 3

### Transwell Assay

### Experimental

Matrigel (Corning, cat. 356234) was polymerized on the insert of the transwell (Corning, cat. 3412) according to manufacturer instructions. 75,000 live A431 cells were stained with CellTrace Violet (Thermo Fischer, cat. C34571) according to manufacturer instructions. The cells were washed with 1 mL ice cold PBS and embedded into the polymerized Matrigel. The cells were allowed to grow overnight. DMEM was added to the top of the transwell to provide the cells with media.

The following day, 75,000 GFP-Mesenchymal stem cells (MSCs) were modified with Alexa-Fluor 647 labeled farnesylated CSANs (500 nM) using the previously mentioned protocol. MSCs were added to the top of the transwell. SDF-1α was embedded into polymerized Matrigel on the bottom of the transwell insert. The transwell plate was allowed to incubate at 37° C. for 24 hrs. The cells were removed from Matrigel using coming cell recovery solution on ice for 1 hr. The cells were then analyzed using flow cytometry.

### Rationale and Key Findings

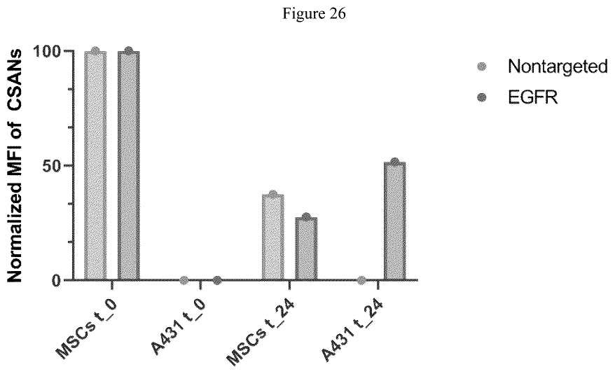

Based on initial findings of CSANs transferring from sender cells (MSCs) to receiver cells (A431), a transwell assay was performed to determine if farnesylated CSANs would still transfer under conditions more similar to in vivo. A431 cancer cells, which express high levels of EGFR, were embedded in the transwell insert using Matrigel. Then, MSCs modified with CSANs were added to the top chamber of the transwell. After 24 hrs it was discovered that farnesylated CSANs do transfer from MSCs to A431 cancer cells when the αEGFR targeting moiety is appended onto the CSANs. Importantly the CSANs that do not possess the targeting moiety, nontargeted CSANS, do not transfer to target cancer cells (FIG. 26). The MFI of CSANs in both treatments does decrease on the MSCs over the course of 24 hrs, likely due to the stability of the fluorophore. However, only targeted CSANs transfer to A431 cells at a 50% increase in CSAN MFI from timepoint 0 after 24 hrs

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

What is claimed is:

1. A conjugate A1 comprising:
A. a fusion protein comprising
i. a targeting domain,
ii. a first dihydrofolate reductase (DHFR) operably linked to a second DHFR, and
iii. a prenyltransferase substrate domain;
B. a linker A operably linked to the prenyltransferase substrate domain of the fusion protein; and
C. a therapeutic agent or a detectable agent operably linked to linker A with a linker B; or
a conjugate A1 comprising
A. a fusion protein comprising
i. a targeting domain,
ii. a first dihydrofolate reductase (DHFR) operably linked to a second DHFR, and
B. a therapeutic agent or a detectable agent operably linked to the DHFR (e.g., the second DHFR) with a linker B.

2. The conjugate of claim 1, wherein the targeting domain is a human tenth type III fibronectin (Fn3) or an affibody.

3. The conjugate of claim 1, wherein the targeting domain has affinity for a cell surface protein.

4. The conjugate of claim 1, wherein the prenyltransferase substrate domain comprises an amino acid sequence of Cys-Val-Ile-Ala (CVIA).

5. The conjugate of claim 1, wherein the linker A is a branched or unbranched, saturated or unsaturated, hydrocarbon chain having from 2 to 50 carbon atoms (lipid) or is selected from a group consisting of a geranyl group, a farnesyl group, a geranylgeranyl group, a palmitate group and a myristyl group.

6. The conjugate of claim 1, wherein linker B is:
—W—Z-T-Y—
wherein:
W is selected from the group consisting of absent, a divalent 6-10 membered aryl or 5-20 membered heteroaryl (e.g., triazolyl or 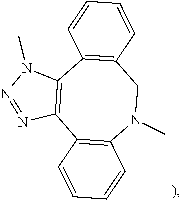 [SMILES: CN1Cc2ccccc2-c2c(nnn2C)-c2ccccc21]
—O—, —S—, —C(═O)—, —N(Rₐ)—, —C(═O)NH—, —C(═S)NH—, —C(═O)O—, —C(═O)S—, —NHSO₂—, —OC(═O)NH—, —NHC(═O)NH—, and —NHC(═S)NH—, wherein Rₐ is H or (C₁-C₆)alkyl;
Z is selected from the group consisting of absent, a peptide, or a divalent, branched or unbranched, saturated or unsaturated, hydrocarbon chain having from 2 to 30 carbon atoms, wherein one or more of the carbon atoms is optionally replaced by (—O—), (—S—), —N(R_{b})—, wherein R_{b} is H or (C₁-C₆)alkyl, wherein the hydrocarbon chain is optionally substituted on carbon with one or more substituents selected from the group consisting of halo, hydroxy, mercapto, oxo(═O), and thioxo(═S);
T is selected from the group consisting of absent, a peptide, or a divalent, branched or unbranched, saturated or unsaturated, hydrocarbon chain having from 2 to 30 carbon atoms, wherein one or more of the carbon atoms is optionally replaced by (—O—), (—S—), —N(R_{c})—, wherein R_{c} is H or (C₁-C₆)alkyl, wherein the hydrocarbon chain is optionally substituted on carbon with one or more substituents selected from the group consisting of halo, hydroxy, mercapto, oxo(═O), and thioxo(═S); and
Y is selected from the group consisting of absent, p-aminobenzyloxycarbonyl, —O—, —S—, —C(═O)—, —N(R_{d})—, —C(═O)NH—, —C(═S)NH—, —C(═O)O—, —C(═O)S—, —NHSO₂—, —OC(═O)NH—, —NHC(═O)NH—, and —NHC(═S)NH—, wherein R_{d} is H or (C₁-C₆)alkyl;
wherein W, Z, T, and Y are not simultaneously absent.

7. The conjugate of claim 1, wherein the linker linking the therapeutic agent or detectable agent to the lipid is:

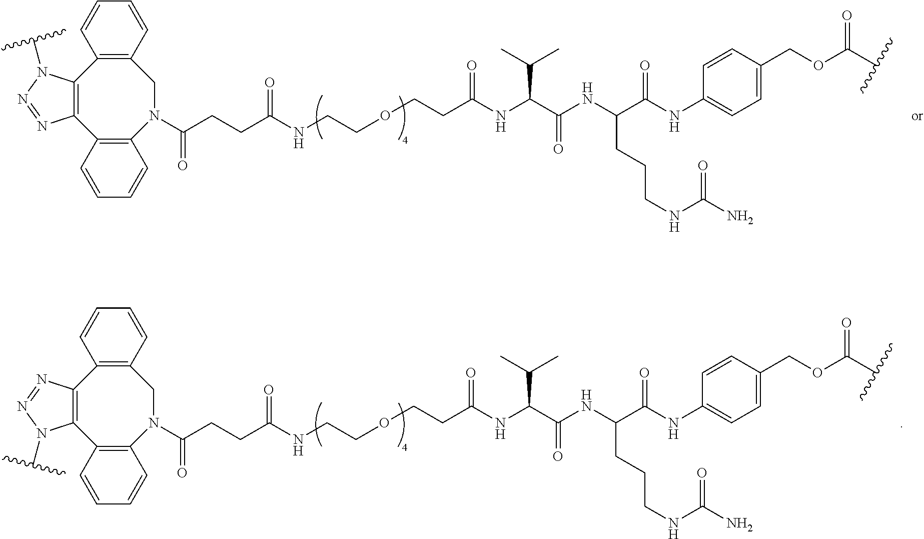

8. The conjugate of claim 1, wherein C is an anti-cancer agent.

9. The conjugate of claim 1, wherein C is a nucleic acid sequence.

10. The conjugate of claim 1, wherein the targeting domain has affinity for a surface protein expressed by a cancer cell.

11. The conjugate of claim 1, wherein the targeting domain has affinity for Her2, EpCAM, or EGFR.

12. A chemically self-assembled nanoring (CSAN) comprising a plurality of conjugates as described in claim 1 and a plurality of bisMTX compounds.

13. The chemically self-assembled nanoring (CSAN) of claim 12, further comprising a plurality of conjugate A2, wherein conjugate A2 comprises
A. fusion protein comprising
i. a targeting domain,
ii. a first dihydrofolate reductase (DHFR) operably linked to a second DHFR, and
iii. a prenyltransferase substrate domain; and
B. a lipid operably linked to the prenyltransferase substrate domain of the fusion protein.

14. A chemically self-assembled nanoring (CSAN) comprising a plurality of conjugate B1 and fusion protein B2, and a plurality of bisMTX compounds,
wherein conjugate B1 comprises:
A. fusion protein comprising
i. a first dihydrofolate reductase (DHFR) operably linked to a second DHFR, and
ii. a prenyltransferase substrate domain; and
B. a lipid operably linked to the prenyltransferase substrate domain of the fusion protein;
wherein conjugate B1 lacks a targeting domain; and
wherein fusion protein B2 comprises:
i. a targeting domain, and
ii. a first dihydrofolate reductase (DHFR) operably linked to a second DHFR.

15. A pharmaceutical composition comprising a conjugate of claim 1 and a pharmaceutically acceptable excipient.

16. A pharmaceutical composition comprising a CSAN of claim 12 and a pharmaceutically acceptable excipient.

17. A cell comprising a CSAN of claim 12 in the cell membrane.

18. A method of intercellular delivery comprising
a) contacting a sender cell with a CSAN of claim 12, and
b) contacting the CSAN modified sender cell with a receiver cell,
wherein a therapeutic agent or a detectable agent linked to a lipid comprised within the CSAN is delivered from the sender cell to the receiver cell.

19. A method for treating cancer in an animal in need of, comprising administering a therapeutically effective amount of a CSAN modified sender cell as described in claim 18 to the animal.

20. A method for monitoring cell-cell interaction and/or intercellular cargo transfer, comprising a) contacting a sender cell with a CSAN as in claim 18, b) contacting the CSAN modified sender cell with a receiver cell, and 3) detecting a signal of a detectable agent comprised within the CSAN.
